SEQUENCE LISTING (90 sequences)

SEQ ID NO: 1 - skipped

SEQ ID NO: 2 - protein, 4 aa, synthetic construct
LPET

SEQ ID NO: 3 - protein, 5 aa, synthetic construct
  VARIANT 3: note = any amino acid
LPXTG

SEQ ID NO: 4 - protein, 16 aa, synthetic construct
LPETGGHHHHHHEPEA

SEQ ID NO: 5 - protein, 143 aa, synthetic construct
AHVQLVESGGELVQAGGSLRLSCAASGRTFSNYAMGWFRQAPGTEREFVAAISRSGGSTYYADSVKGRFIISRDNAKNTVWLQMNMLKPEDTSVYYCAAAEGLASGSYDYAPPLKSSWYDYWGQGTQVTVSEPKTPKGGCGGG

SEQ ID NO: 6 - protein, 135 aa, synthetic construct
AEVQLVESGGGLAQAGGSLRLSCAASGRTFSNECLGWFRQAPGKEREFVATIRSTGHTSYADAVSGRFTVSRDIAKNTVYLEMSNLKPEDTAVYSCAAGFSDYGCYRTSGINYWGQGTQVTVSEPKTPKGGCGGG

SEQ ID NO: 7 - protein, 143 aa, synthetic construct
AQVQLVESGGGLVQAGGSLRLSCTASGRTFSSYYAMGWFRRAPGKEREFVAAISESGRTTDYADSVKGRFTISRDTAKNTVYLQMISLKPEDTAVYYCAAAGPQEAFWFPSDYAQRALYDYWGQGTQVTVSEPKTPKGGCGGG

SEQ ID NO: 8 - protein, 136 aa, synthetic construct
AQVQLVESGGGLVQAGDSLRLSCAASERTFGAHVMGWFRQAPGKEREFVATITSSGRNTRYADSVKGRFTISSDNAKNTVYLQMISLEPEDTAVYYCAYAYGAGLYNIARQYDYWGQGTQVTVSEPKTPKGGCGGG

SEQ ID NO: 9 - protein, 136 aa, synthetic construct
AHVQLVESGGGLMQAGDSLRLSCAASGLTFSNYAMGWFRQAPGREREFVAALSWSGRNGYYADSVKGRFTISSDNAKNTVYLQMNSLKPEDTAIYYCASAGGGGLYKIATQYDYWGQGTQVTVSEPKTPKGGCGGG

SEQ ID NO: 10 - protein, 130 aa, synthetic construct
AQVQLVESGGGLVQPGGSLRLSCVASGIMFDIYTMRWYRQAPGKQRELVAAITGAGRANYNDDSVKGRFTISRDNAKNTVYLQMNRMKPEDTALYECNTEILGGGPNYWGRGTQVTVSEPKTPKGGCGGG

SEQ ID NO: 11 - protein, 135 aa, synthetic construct
ADVQLVESGGGLAQAGGSLRLSCAASGRTFSNSCMGWFRQAPGKEREFVVTIRSTGHTSYADAVSGRFTVSRDIAKNTVYLEMNSLKPEDTAVYSCGAGVSDYGCYHTSGYKYWGQGTQVTVSEPKTPKGGCGGG

SEQ ID NO: 12 - protein, 135 aa, synthetic construct
AHVQLVESGGGLAQAGGSLRLSCAASGGTFSNSCMGWFRQAPGMEREFVATIRSTGHTTYADSVEGRFTVSRDIAKNTVYLEMNSLKPEDTAVYSCGAGISDYGCYRTSGYNYWGQGTQVTVSEPKTPKGGCGGG

SEQ ID NO: 13 - protein, 136 aa, synthetic construct
AQVQLVESGGGLVQAGGSLRLSCRASGLPFGPYTMGWFRQTPGQEREFVAAITWSSMNTNYADSVKGRFTISRDSAKNTVYLQMNTLKPDDTAVYYCAAAPGVGYYRHTFQYDYWGQGTQVTVSEPKTPKGGCGGG

SEQ ID NO: 14 - protein, 136 aa, synthetic construct
AHVQLVESGGGLVQAGGSLRVSCAASGREFSNYGMGWFRQAPGKEREFVATISWNGRITFYADSVKGRFTISRDNAEKTGYLQMNSLKPEDTALYYCAAETSGWGSKVVPNYDYWGQGTQVTVSEPKTPKGGCGGG

SEQ ID NO: 15 - protein, 140 aa, synthetic construct
AQVQLVESGGGLVQAGGSLRLSCTIPGHTISSYIMGWFRQAPGKEREFVAAINWSGGRTNSADSVKGRFTISRDNAKNTVYLQMNSLKPEDTAVYYCAAVFDRISDSALPEERSRYDYWGQGTQVTVSEPKTPKGGCGGG

SEQ ID NO: 16 - protein, 135 aa, synthetic construct
ADVQLVESGGGLAQAGGSLRLSCAASGRTFSNSCMGWFRQAPGKEREFVATIRSTGHITYADSVEGRFTVSRDIAKNTVYLEMSNLKPEDTAVYSCGAGVSDYGCYRTSGYNYWGQGTQVTVSEPKTPKGGCGGG

SEQ ID NO: 17 - protein, 138 aa, synthetic construct
AQVQLVESGGGLVQAGASLRLSCAASGGTFSSYIMGWFRQAPGREREFVAAISWSGRSTHYADSVKGRFAISRDNDRVYLQMDSLKPEDTAVYSCAADPNYTWRDDRYYREEGYTYWGQGTQVTVSEPKTPKGGCGGG

SEQ ID NO: 18 - protein, 140 aa, synthetic construct
AQVQLVESGGGLVQSGGSLRLSCAASGSIGVTNTMGWYRQAPGKQRELAATITNDGNTNYADSVKGRFTISRDNAKNTVYLQMNSLKPEDTAVYYCAGAGKRIPVSTMGWANDNEYEYWGRGTQVTVSEPKTPKGGCGGG

SEQ ID NO: 19 - protein, 137 aa, synthetic construct
AQVQLVESGGGLVQAGGSLRVSCAASGSTFSNYGMGWFRQAPGKEREFVAVIAWIGGKTDYSDSVKGRFTIFRDNAKNTVYLQMNSLKPEDTAVYYCAATSYGTISRRSEYEYGYWGQGTQVTVSEPKTPKGGCGGG

SEQ ID NO: 20 - protein, 137 aa, synthetic construct
AQVQLVESGGGLVQAGGSLRLSCAASGRTFSTYHMGWFRQAPGKAREFVAAITENGGITYYADSVRGRFTISRDDARNTVYLQMGSLKPEDTAVYYCAASSALIGRKYFGNENYSWGQGTQVTVSEPKTPKGGCGGG

SEQ ID NO: 21 - protein, 137 aa, synthetic construct
AQVQLVESGGGLVQAGGSLRLSCAASGDTFSTYGVAWFRQAPGKERELVAITPWMGSSTYYADSVKGRFTISRDNAKNTVYLQMNSLKPEDTAVYYCAASSYGSISRRSDYEYGYWGQGTQVTVSEPKTPKGGCGGG

SEQ ID NO: 22 - protein, 137 aa, synthetic construct
AEVQLVESGGGLVQAGGSLRLSCAASGDTFGTYGVAWFRQAPGKEREFVAVTPWMGSNTYYADSMKGRFTISRDNAKNTVYLQMNSLKPEDTAVYYCAASSYGTVSRRSEYEYGYWGQGTQVTVSEPKTPKGGCGGG

SEQ ID NO: 23 - protein, 136 aa, synthetic construct
AQVQLVESGGGLVQAGDSLRLSCAASGRTFETHAMGWFRQAPGKEREFVATITPSGRSTSYGDSVKGRFTISSDNAKNTVYLQMNSLKPDDTAIYYCAFAYGVGLYKLARQYDYWGQGTQVTVSEPKTPKGGCGGG

SEQ ID NO: 24 - protein, 136 aa, synthetic construct
AQVQLVESGGGLVQAGDSLRLSCAASGRTFSTHGTGWFRQAPGKEREFVATITSSGRSTSYADSTKGRFTISSDNAKNTVYLQMNSLKPEDTAIYYCAYAYGVGLYKFATQYDYWGQGTQVTVSEPKTPKGGCGGG

SEQ ID NO: 25 - protein, 136 aa, synthetic construct
AQVQLVESGGGLVQAGSSLRLSCAASGSTFSSHGMGWFRQAPGKEREFVATVSLSGRTTSYGDSVKDRFTISRDNAKNTVYLHMNSLKLEDTAVYYCAATSGGYYSRYAYDYYYWGQGTQVTVSEPKTPKGGCGGG

SEQ ID NO: 26 - protein, 136 aa, synthetic construct
AQVQLVESGGGLVQAGDSLRLSCAASGRTFETHAMGWFRQAPGKEREFVATITPSGRSTSYADSVKGRYTISSDNAKNTVYLQMDSLKPEDTAIYYCAFAYGVGLYKIARQYDYWGQGTQVTVSEPKTPKGGCGGG

SEQ ID NO: 27 - protein, 136 aa, synthetic construct
AEVQLVESGGGLVQAGGSLRLSCAASGRTYSPLVMGWFRQAPGKEREFVATITPSGGSLSYADSVTGRFTVSRDNAKKTVFLQMNSLKPEDTAIYYCAAAPGVGNYRYARQYDYWGQGTQVTVSEPKTPKGGCGGG

SEQ ID NO: 28 - protein, 136 aa, synthetic construct
AQVQLVESGGGLVQAGGSLRLSCTASGRTFTPYTMGWFRQAPGKEREFAASILWSGKNTDYADSVKGRFAISKDNAKNTVYLQMNKLKPEDTAVYYCATGDGLGFYRSVSQYDYWGQGTQVTVSEPKTPKGGCGGG

SEQ ID NO: 29 - protein, 136 aa, synthetic construct
AQVQLVESGGGLVQAGGSLRLSCTASGRTYEPLVMGWFRQAPGKEREFVATITPSGGSLSYADSVKGRFTVSRDNAKKTVYLQMNRLQPEDTAVYYCAAAPGVGNYRYTRQYDYWGQGTQVTVSEPKTPKGGCGGG

SEQ ID NO: 30 - protein, 136 aa, synthetic construct
AQVQLVESGGGLVQAGGSLRLSCTASGRTFTPYTIGWFRQAPGKEREFVASILWSGINTDYADSVKGRFAISRDNAKNAAYLQMSNLKPEDTAVYYCATGGGLGYYRSVSQYDYWGQGTQVTVSEPKTPKGGCGGG

SEQ ID NO: 31 - protein, 136 aa, synthetic construct
AEVQLVESGGGLVQAGGSLRLSCTASGRTFTPYTMGWFRQAPGKEREFVASILWSGNNRDYADSVKGRFAISRDNAKNTAYLQMTSLKPEDTAVYYCAAGDGLGFYRSVNQYDYWGQGTQVTVSEPKTPKGGCGGG

SEQ ID NO: 32 - protein, 136 aa, synthetic construct
AQVQLVESGGGLVQAGGSLKLSCTASGRTFMPYTMGWFRQVPGKEREFVASVLWSGINTDYAESVKGRFAISKDNAKNTMYLQMNSLKPEDTAVYYCAAGDGLGYYRSVSQYDYWGHGTQVTVSEPKTPKGGCGGG

SEQ ID NO: 33 - protein, 136 aa, synthetic construct
AHVQLVESGGGLVQAGGSLRLSCAASGRTFSTYHMGWFRQAPGKAREFVAAITESGGITYYADSVKGRFTVSRDNAKNTVDLQMNSLKPEDTAVYYCAAAPGVGAYRHATQYDYWGQGTQVTVSEPKTPKGGCGGG

SEQ ID NO: 34 - protein, 136 aa, synthetic construct
AQVQLVESGGGLVQAGGSLRLSCVASGRTFEPFVMGWFRQAPGKEREFVATISWSGGSLSYADSVKGRFTVSRDNAKNTVYLQMNSLKPEDTAVYYCAAAPGVGNYRYTFQYDYWGQGTQVTVSEPKTPKGGCGGG

SEQ ID NO: 35 - protein, 136 aa, synthetic construct
AQVQLVESGGGLVQAGGSLRLSCTASGRTFTPYTMGWFRQTPGKEREFAASILWSGINTDYADSVKGRFAISKDNAKNTVYLQMNSLKPEDTAVYYCAAAYGLGYYRSVSQYDYWGQGTQVTVSEPKTPKGGCGGG

SEQ ID NO: 36 - protein, 136 aa, synthetic construct
AQVQLVESGGGLVQAGGSLRLSCTASGRTFTPYTMGWFRQAPGKEREFAASILWSGENTDYADSVKGRFAISRDGAKNTVYLQMNSLKPEDTAVYYCASGYGLGFYRSASQYDYWGQGTQVTVSEPKTPKGGCGGG

SEQ ID NO: 37 - protein, 136 aa, synthetic construct
AQVQLVESGGGLVQAGGSLRLSCTASGRTFTPYTMGWFRQAPGKEREFAASILWSGINTDYADSVKGRFAISRDNAKNTVYLQMNSLKPEDTGVYYCAAADGLGLYRFVSQYDDWGQGTQVTVSEPKTPKGGCGGG

SEQ ID NO: 38 - protein, 136 aa, synthetic construct
AQVQLVESGGGLVQAGGSLRLSCTASGRTFTPYTMGWFRQAPGKEREFAASILWSGINTDYADSVKGRFAISRDNAKNTVYLQMNSLKPEDTGVYYCATADGLGLYRFVSQYDYWGQGTQVTVSEPKTPKGGCGGG

SEQ ID NO: 39 - protein, 136 aa, synthetic construct
AQVQLVESGGGLVQAGGSLRLSCAASGRTFSPLVMGWFRQAPGHEREFVATITPSGGSQSYADSVKGRFAVSRDNAKKTVYLQMNSLKPEDTAVYYCAAAPGVGIYRYTSQYDYWGQGTQVTVSEPKTPKGGCGGG

SEQ ID NO: 40 - protein, 136 aa, synthetic construct
AQVQLVESGGGLVQPGKSLRLSCTTSGLPGSWYTLGWFRQVPGKEREFVASVLWSGINTDYADSVKGRFAISRNNAKNTMYLQMNSLKPEDTAVYYCAAGYGLGFYRSVSQYDYWGHGTQVTVSEPKTPKGGCGGG

SEQ ID NO: 41 - protein, 135 aa, synthetic construct
AQVQLVESGGGLVQAGGSLRLSCSASGSTFSPFVIGWFRQAPGKEREFVGGVRPSGSQYYSDSVKGRFTVSRDNAKNTVYLQMNSLKPEDTAVYYCAAAAGVGNYRHTWQYDYWGQGTQVTVSEPKTPKGGCGGG

SEQ ID NO: 42 - protein, 135 aa, synthetic construct
AHVQLVESGGGLVQAGGSLRLSCAASGHTFGPYTMGWFRQTPGKEREFVAAITWSGTSTNYADSVKGRFTISRDNAKNTAYLQMNSLKPEDTAVYYCAAGSGAGRTSMHTSMTTGARGPRSPSEPKTPKGGCGGG

SEQ ID NO: 43 - protein, 136 aa, synthetic construct
AQVQLVESGGGLVQAGDSLRLSCVASGRTFSTYHMGWFRQAPGKAREFVAAITQSGITYYADSVKGRFTISRDNAKNTAYLQMGSLQPEDTAVYYCAASPKLIGRIYFGNENYSWGQGTQVTVSEPKTPKGGCGGG

SEQ ID NO: 44 - protein, 137 aa, synthetic construct
AQVQLVESGGGLVQAGGSLRLSCAASGRTFSTYHMGWFRQAPGKAREFVAAITQSGGITYYADSVKGRFTISRDDAKNTVYLQMGSLEPEDTAVYYCAASPTLIGRVYFGNENYSWGQGTQVTVSEPKTPKGGCGGG

SEQ ID NO: 45 - protein, 137 aa, synthetic construct
AQVQLVESGGGLVQAGGSLRLSCAASGRTFSTYHMGWFRQAPGKAREFVAAITGSGGITYYADSVKGRFTISRDNAKNTVYLQMNSLKPEDTAVYYCAASVALIGRVYFGNENYSWGQGTQVTVSEPKTPKGGCGGG

SEQ ID NO: 46 - protein, 137 aa, synthetic construct
AQVQLVESGGGLVQAGGSLRLSCAASGRTFSTYHMGWFRQAPGKEREFVAAITQSGGITYYADAMKGRFTISRDDAKNTVYLQMGSLKPEDTAVYYCVASPALIGRHYFGNENYSWGQGTQVTVSEPKTPKGGCGGG

SEQ ID NO: 47 - protein, 137 aa, synthetic construct
AHVQLVESGGGLVQAGGSLRLSCAASERTFSTYHMGWFRQAPGKGREFVAAITPSGGVTYYADNLKGRFTISGDNAKNTVYLQMTNLKPEDTAVYYCVASPALIGRVYFGNENYSWGQGTQVTVSEPKTPKGGCGGG

SEQ ID NO: 48 - protein, 135 aa, synthetic construct
AEVQLVESGGGLAQAGGSLRLSCAASGRTFSNSCMGWFRQAPGKEREFVATIRSTGHASYADSVEGRFTVSRDIAKNTVYLEMNSLKPDDTAVYICGAGVSDYGCYRTSGYNSWGQGTQVTVSEPKTPKGGCGGG

SEQ ID NO: 49 - protein, 135 aa, synthetic construct
AQVQLVESGGGLAQAGGSLRLSCAASGGTFSNNCMGWFRQAPGMEREFVAIIRSTGHTTYADSVEGRFTVSRDIAKNTVYLEMNSLKPEDTAVYICAAGASDYGCYRTSGINYWGQGTQVTVSEPKTPKGGCGGG

SEQ ID NO: 50 - protein, 135 aa, synthetic construct
AHVQLVESGGGLVQTGGSLRLSCVASGGIFSNSCMGWFRQAPGMERQFVAIIRSTGHTTYADSVEGRFTVSRDIAKNTVYLEMNSLKPEDTAVYYCAAGVSDYGCYRTSGINYWGQGTQVTVSEPKTPKGGCGGG

SEQ ID NO: 51 - protein, 136 aa, synthetic construct
AQVQLVESGGGLVQAGGSLRLSCAASGRTFSTYHMGWFHQAPGKAREFVAAITESGGITYYADSVKGRFTISRDIAKNTVNLEMNSLKPEDTAVYSCAAGISDYGCYRTSGIAYWGQGTQVTVSEPKTPKGGCGGG

SEQ ID NO: 52 - protein, 140 aa, synthetic construct
AQVQLVESGGGLVQAGGSLRLSCAASGFSFSSYGMGWFRQAPGKEREFVAAIGWIGSRTSYADSVKGRFTISKDNAKNTVYLQMDSLRPEDTAVYTCAATSYLNPDSDYARSDRSYGYRGQGTQVTVSEPKTPKGGCGGG

SEQ ID NO: 53 - protein, 146 aa, synthetic construct
AEVQLVESGGGLVQAGDSLRLSCVASGRTFSTYHMGWFRQAPGKAREFVAAITQSGITYYADSVKGRFTISRDNAKNTAYLQMGSLQPEDTAVYYCAASPLLIGRVYFGNEDYSWGPQRISEATTGARGPRSPSEPKTPKGGCGGG

SEQ ID NO: 54 - protein, 136 aa, synthetic construct
AQVQLVESGGGLVQAGGSLRLSCAASGRTFSTYHMGWFRQAPGKAREFVAAITGSGGITYYADSVKGRFTISRDNAKNTVYLQMNSLKPEDTAVYYCAASAALIGRVYFGNEITPGARGPRSPSEPKTPKGGCGGG

SEQ ID NO: 55 - protein, 135 aa, synthetic construct
AQVQLVESGGGLVQAGGSLRLSCAASERTFSSYAMGWFRQGPGKEREFVAYIHWSGGRTLVVDSVKGRFTISRDNTKNTMYLQMNSLKPADTAVYYCTADQYASTLLRGTGEYWGQGTQVTVSEPKTPKGGCGGG

SEQ ID NO: 56 - protein, 135 aa, synthetic construct
AQVQLVESGGGLVQAGGSLRLSCAAPGDIFSMYVMGWFRQAPGKEREFVAYNHWSGGRTLYADSVKGRFTISRDNSKNTMSLQMNSLRPEDTAVYYCTADQYASTLLRAAGEYWGQGTQVTVSEPKTPKGGCGGG

SEQ ID NO: 57 - protein, 135 aa, synthetic construct
AQVQLVESGGGLVQAGGSLRLSCAASGLTFSNYVMGWFRQAPGKEREFVAYIHWSGGRILYADSVKGRFTISRDNTKNTMYLQMNSLKPDDTAVYYCTADQYATTVLRAAGEYWGQGTQVTVSEPKTPKGGCGGG

SEQ ID NO: 58 - protein, 136 aa, synthetic construct
AQVQLVESGGGLVQAGGSLRLSCVASGRTFSPYTTGWFRQAPGKEREFVAAITWSGRSTNYAASVKGRFTISRDNAKNTVYLQMDSLKPEDTAVYYCSAGAGGGIYTIRGQYDYWGQGTQVTVSEPKTPKGGCGGG

SEQ ID NO: 59 - protein, 134 aa, synthetic construct
AHVQLVESGGGLVQAGGSLRLSCAASGGTISNYGMGWLRQGPGKEREFVGSINWNGATTHYADSVKGRFIISRDNAKNTVYLQMNSLKPEDTGVYYCVAQFSVQPTLRTYDYGGQGTQVTVSEPKTPKGGCGGG

SEQ ID NO: 60 - protein, 138 aa, synthetic construct
AHVQLVESGGGLVQAGGSLRLSCAASGLTFRNYAMGWFRRAPGKERDFVAAISYSGGSTDYADSVKGRFTISRDNAKNTVFLQMSSLKPEDTAVYYCAASPVVYGSLWFKRESYTYWGQGTQVTVSEPKTPKGGCGGG

SEQ ID NO: 61 - protein, 143 aa, synthetic construct
ADVQLVESGGGLVQAGGSLRLSCRASGLPFGPYTMGWFRQTPGQEREFVAAITWSSMNTNYADSVKGRFTISRDNAKNTVLLQMNSLKPEDTAVYYCAAAEGLASGSYDYFPPLKSSWYDYWGQGTQVTVSEPKTPKGGCGGG

SEQ ID NO: 62 - protein, 138 aa, synthetic construct
AQVQLVESGGGLVQPGGSLRLSCAASGFTLDYYAIGWFRQAPGKEREGVGCISSSDGSPTYADSVKGRFTISRDNAKNTVYLQMNSLKPEDTAVYYCATHFRSANSCFLYYYDMDYSGKGTQVTVSEPKTPKGGCGGG

SEQ ID NO: 63 - protein, 136 aa, synthetic construct
AQVQLVESGGGLVQAGDSLRLSCAASGRTFVAHAMGWFRQAPGKERTFVAMITSSGLTISYADPVKGRFTISSDNAKNTVYLQMNSLKPEDTAIYYCAFAYGVGKYEIARQYDYWGQGTQVTVSEPKTPKGGCGGG

SEQ ID NO: 64 - protein, 135 aa, synthetic construct
AQVQLVESGGGAVQAGGSLQLSCRASGRTFSPYVMGWFRQAPGKEREFVGLITWSGGTSYADSVRGRFTASRDRVKNTVYLQMNSLKPEDTAVYYCAAAYGAGYYVHERQYDYWGQGTQVTVSEPKTPKGGCGGG

SEQ ID NO: 65 - protein, 140 aa, synthetic construct
AQVQLVESGGGLVQAGGSLRLSCTIPGHTISSYIMGWFRQAPGKEREFVAAINWSGGRTNSADSVKGRFTISRDNAKNTVYLQMNSLKPEDTAVYYCAAVFDRLSDSALPEERSRYDYWGQGTQVTVSEPKTPKGGCGGG

SEQ ID NO: 66 - protein, 139 aa, synthetic construct
AEVQLVESGGGLAQAGGSLRLSCAASGGTFSNSCMGWFRQAPGMEREFVAIIRSTGHTTYADSVEGRFTISRDNAKNTVYLEMNSLKPEDTAVYYCAAERWTGACSGAGLHLRSFTSWGQGTQVTVSEPKTPKGGCGGG

SEQ ID NO: 67 - protein, 141 aa, synthetic construct
AQVQLVESGGGLVQAGGSLSVSCAASGRTFRSYVGWFRQAPGKERTFVAGIRWSAGDTYYADSMKGRFTISRDSAKNTVYLQMNSLKPEDTAVYYCAAAGPQQAFWFPSDYAQRALYDYWGQGTQVTVSEPKTPKGGCGGG

SEQ ID NO: 68 - protein, 142 aa, synthetic construct
AHVQLVESGGGLVQAGGSLRLSCAASGRTFIPYTTGWFRQTPGKEREFVATITWSGISTKFADSVKGRFTISRDNAKNTVYLQMNSLKPEDTAVYYCAASGPQEAFWFPSDYAQRALYDYWGQGTQVTVSEPKTPKGGCGGG

SEQ ID NO: 69 - protein, 142 aa, synthetic construct
AQVQLVESGGGLVQAGGSLRLSCRASGLPFGPYTMGWFRQTPGQEREFVAAITWSSMNTNYADSVKGRFTISRDSAKNTVYLQMNSLKREDTAVYYCAAAGPQEAFWFPSDYAQRALYDYWGQGTQVTVSEPKTPKGGCGGG

SEQ ID NO: 70 - protein, 130 aa, synthetic construct
AHVQLVESGGGLVQPGGSLRLSCVASGIMFDIYTMRWYRQAPGKQRELVAAITGAGRANYNDDSVKGRFTISRDNAKNTVYLQMNRMKPEDTALYECNTEILGGGRNYWGRGTQVTVSEPKTPKGGCGGG

SEQ ID NO: 71 - protein, 138 aa, synthetic construct
AQVQLVESGGGLVQAGDSLRLSCAVSGRAFSNDIFGWFRQAPGLEREFVAAHRWNALYADSVKGRFTISRDNAKNTVYLQMNSLKPEDTAVYFCAGAGKRFPVSAMGWANDNEYEYWGRGTQVTVSEPKTPKGGCGGG

SEQ ID NO: 72 - protein, 138 aa, synthetic construct
AHVQLVESGGGLVQAGGSLRLSCAVSGRTFSNDIIGWFRQAPGKDREFVAAHRYNALYADSVKGRFTISRDNAKNTVYLQMNSLKPEDTALYYCAGAGKRYPVSTMGWANDNEYEYWGRGTQVTVSEPKTPKGGCGGG

SEQ ID NO: 73 - protein, 135 aa, synthetic construct
AQVQLVESGGGLAQAGGSLRLSCAASGGTFSNSCMGWFRQAPGMEREFVATIRSTGHTTYADSVEGRFTVSRDIAKNTVYLEMNSLKREDTAVYTCAAGVSDYGCYHTSGYNYWGQGTQVTVSEPKTPKGGCGGG

SEQ ID NO: 74 - protein, 140 aa, synthetic construct
AQVQLVESGGGLVQAGGSLRLSCTVPGHTISSYIMGWFRQAPGKEREFVAAINWNGGRTNSADSVKGRFTISRDNAKNTVYLQMNSLKPEDTAVYYCAAVFDRISDSFLPEERSTYDYWGQGTQVTVSEPKTPKGGCGGG

SEQ ID NO: 75 - protein, 134 aa, synthetic construct
ADVQLVESGGGLVQAGGSLRLSCAASGGTISNYGMGWLRQGPGKEREFVGSINWNGATTHYADSVKGRFIISRDNAKNTVYLQMNSLKPEDTGVYYCVAQFSVQPTLQTYDYRGQGTQVTVSEPKTPKGGCGGG

SEQ ID NO: 76 - protein, 131 aa, synthetic construct
EVQLVESGGGLVQPGGSLRLSCAASGFTFRSFGMSWVRQAPGKEPEWVSSISGSGSDTLYADSVKGRFTISRDNAKTTLYLQMNSLKPEDTAVYYCTIGGSLSRSSQGTQVTVSSLPETGGHHHHHHEPEA

SEQ ID NO: 77 - protein, 140 aa, synthetic construct
QVQLVESGGGFVQAGGSLRLSCAASGRTFDNYVMAWFRQAPGKEREFVASISGSGSITNYANSVKDRFTISRDSAKNAIYLQMNSLKPEDTALYYCAAGSRRTYYREPKFYPSWGQGTQVTVSSLPETGGHHHHHHEPEA

SEQ ID NO: 78 - protein, 378 aa, synthetic construct
EVQLVESGGGLVQPGGSLRLSCAASGFTFRSFGMSWVRQAPGKEPEWVSSISGSGSDTLYADSVKGRFTISRDNAKTTLYLQMNSLKPEDTAVYYCTIGGSLSRSSQGTQVTVSSSGSETPGTSESAVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKLPETGGHHHHHHEPEA

SEQ ID NO: 79 - protein, 387 aa, synthetic construct
QVQLVESGGGFVQAGGSLRLSCAASGRTFDNYVMAWFRQAPGKEREFVASISGSGSITNYANSVKDRFTISRDSAKNAIYLQMNSLKPEDTALYYCAAGSRRTYYREPKFYPSWGQGTQVTVSSSGSETPGTSESAVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKLPETGGHHHHHHEPEA

SEQ ID NO: 80 - protein, 155 aa, synthetic construct
QAKPQIPKDKSKVAGYIEIPDADIKEPVYPGPATREQLNRGVSFAEENESLDDQNISIAGHTFIDRPNYQFTNLKAAKKGSMVYFKVGNETRKYKMTSIRNVKPTAVEVLDEQKGKDKQLTLITCDDYNEETGVWETRKIFVATEVKLEHHHHHH

SEQ ID NO: 81 - protein, 140 aa, synthetic construct
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSLPETGGHHHHHHEPEA

SEQ ID NO: 82 - protein, 132 aa, synthetic construct
QVQLQESGGGLVHPGGSLRLSCATSGSIFSIISMGWYRQAPGKQRELVALVFRGGSTVYADSVKGRFTISGDIAKSTVYLQMDSLKPEDTAVYYCNAKPIGTAQYWGQGTQVTVSSLPETGGHHHHHHEPEA

SEQ ID NO: 83 - protein, 259 aa, synthetic construct
EVQLVESGGGLVQPGGSLRLSCAASGFTFRSFGMSWVRQAPGKEPEWVSSISGSGSDTLYADSVKGRFTISRDNAKTTLYLQMNSLKPEDTAVYYCTIGGSLSRSSQGTQVTVSSSGSETPGTSESAQVQLQESGGGLVHPGGSLRLSCATSGSIFSIISMGWYRQAPGKQRELVALVFRGGSTVYADSVKGRFTISGDIAKSTVYLQMDSLKPEDTAVYYCNAKPIGTAQYWGQGTQVTVSSLPETGGHHHHHHEPEA

SEQ ID NO: 84 - protein, 12 aa, synthetic construct
SGSETPGTSESA

SEQ ID NO: 85 - protein, 260 aa, synthetic construct
MEVQLVESGGGLVQPGGSLRLSCAASGFTFRSFGMSWVRQAPGKEPEWVSSISGSGSDTLYADSVKGRFTISRDNAKTTLYLQMNSLKPEDTAVYYCTIGGSLSRSSQGTQVTVSSSGSETPGTSESAQEQLKESGGRLVTPGTPLTLTCTVSGFSPNNYGVSWVRQPPGKGLEWIGMSSTAGATYYANWAKGRFTISKTSTTVDLEITSPTTEDTATYFCAKGTPSLSYGNIWGPGTLVTVSSLPETGGHHHHHHEPEA

SEQ ID NO: 86 - DNA, 780 bp, synthetic construct
  source 1..780: mol_type = other DNA
atggaagtgcagctggtggaaagcggcggcggcctggtgcagccgggcggcagcctgcgcctgagctgcgcggcgagcggctttacctttcgcagctttggcatgagctgggtgcgccaggcgccgggcaaagaaccggaatgggtgagcagcattagcggcagcggcagcgataccctgtatgcggatagcgtgaaaggccgctttaccattagccgcgataacgcgaaaaccaccctgtatctgcagatgaacagcctgaaaccggaagataccgcggtgtattattgcaccattggcggcagcctgagccgcagcagccagggcacccaggtgaccgtgagcagcagcggcagcgaaaccccgggcaccagcgaaagcgcgcaggaacagctgaaagaaagcggcggccgcctggtgaccccgggcaccccgctgaccctgacctgcaccgtgagcggctttagcccgaacaactatggcgtgagctgggtgcgccagccgccgggcaaaggcctggaatggattggcatgagcagcaccgcgggcgcgacctattatgcgaactgggcgaaaggccgctttaccattagcaaaaccagcaccaccgtggatctggaaattaccagcccgaccaccgaagataccgcgacctatttttgcgcgaaaggcaccccgagcctgagctatggcaacatttggggcccgggcaccctggtgaccgtgagcagcctgccggaaaccggcggccatcatcatcatcatcatgaaccggaagcg

SEQ ID NO: 87 - protein, 132 aa, synthetic construct
QEQLKESGGRLVTPGTPLTLTCTVSGFSPNNYGVSWVRQPPGKGLEWIGMSSTAGATYYANWAKGRFTISKTSTTVDLEITSPTTEDTATYFCAKGTPSLSYGNIWGPGTLVTVSSLPETGGHHHHHHEPEA

SEQ ID NO: 88 - protein, 116 aa, synthetic construct
QEQLKESGGRLVTPGTPLTLTCTVSGFSPNNYGVSWVRQPPGKGLEWIGMSSTAGATYYANWAKGRFTISKTSTTVDLEITSPTTEDTATYFCAKGTPSLSYGNIWGPGTLVTVSS

SEQ ID NO: 89 - protein, 115 aa, synthetic construct [sequence omitted]

SEQ ID NO: 90 - protein, 116 aa, synthetic construct
QVQLQESGGGLVHPGGSLRLSCATSGSIFSIISMGWYRQAPGKQRELVALVFRGGSTVYADSVKGRFTISGDIAKSTVYLQMDSLKPEDTAVYYCNAKPIGTAQYWGQGTQVTVSS

(19) United States
(12) Patent Application Publication
(10) Pub. No.: US 2026/0216361 A1
(43) Pub. Date: Jul. 30, 2026

(54) NANOBODY-DRUG CONJUGATES AND METHODS OF PREPARING THEREOF

(71) Applicant: Vanderbilt University, Nashville, TN (US)

(72) Inventors: John T. Wilson, Nashville, TN (US); Karan Arora, Nashville, TN (US); Blaise R. Kimmel, Nashville, TN (US)

(21) Appl. No.: 19/130,090

(22) PCT Filed: Nov. 15, 2023
(86) PCT No.: PCT/US2023/079885

§ 371 Date: May 14, 2025

Related U.S. Application Data
(60) Provisional application No. 63/383,856, filed on Nov. 15, 2022.
(60) Provisional application No. 63/472,528, filed on Jun. 12, 2023.
(60) Provisional application No. 63/519,556, filed on Aug. 14, 2023.

(51) Int. Cl. A61K 47/68; A61K 47/60; A61P 35/00

(52) U.S. Cl. CPC A61K 47/6843; A61K 47/60; A61K 47/6849; A61K 47/6889; A61P 35/00

(57) ABSTRACT

Disclosed herein are nanobody-drug conjugates that can effectively deliver drugs, such as STING agonists. An example conjugate includes an albumin-binding nanobody, a drug, and a linker attaching the nanobody to the drug. Also disclosed are methods of making and using the conjugates.

### CROSS-REFERENCE TO RELATED APPLICATIONS

This application claims priority to U.S. Provisional Patent Application No. 63/383,856 filed on Nov. 15, 2022; U.S. Provisional Patent Application No. 63/472,528 filed on Jun. 12, 2023; and U.S. Provisional Patent Application No. 63/519,556 filed on Aug. 14, 2023, each of which are incorporated fully herein by reference.

### FEDERALLY SPONSORED RESEARCH

This invention was made with government support under Grant No. 5R01CA245134 awarded by the National Institutes of Health and the National Cancer Institute. The government has certain rights in the invention.

### TECHNICAL FIELD

This disclosure relates to nanobody-drug conjugates and their use in biomedical applications, such as drug delivery.

### INTRODUCTION

Immune checkpoint inhibitors (ICIs) targeting, e.g., 72 CTLA-4 and PD-1/PD-L1 have improved the treatment of an increasing number of cancers but are still only effective for a relatively small fraction of patients (~15%). For many cancers, this can be attributed, in part, to poor tumor immunogenicity and an immunosuppressive (i.e., “cold”) tumor microenvironment (TME) that can restrict the infiltration and/or function of antitumor T cells. The innate immune system plays a role in cancer immune surveillance, with clinical evidence linking activation of certain pattern recognition receptor (PRR) signaling pathways to increased T cell infiltration and responses to TCTs in cancer patients. Accordingly, the relationship between innate and adaptive antitumor immunity has led to the clinical exploration and continued development of agonists targeting PRRs, including toll-like receptors (TLRs), RIG-I-like receptors (RLRs), and stimulator of interferon genes (STING), which may be able to induce a coordinated antitumor immune response and enhance the efficacy of ICIs. However, therapeutic targeting of PRRs and ICIs remains a significant challenge owing to multiple intertwined pharmacological barriers, including suboptimal pharmacokinetics and poor tumor accumulation, that limit efficacy and increase the risk of inflammatory toxicities.

### SUMMARY

In one aspect, disclosed are conjugates comprising a nanobody portion, the nanobody portion comprising an albumin-binding nanobody, wherein the albumin-binding nanobody is capable of specifically binding albumin; a drug; and a linker attaching the nanobody portion to the drug, the linker comprising a nanobody linking moiety attached to the nanobody portion, the nanobody linking moiety including an amino acid sequence of LPXT (SEQ ID NO: 1), wherein X is any amino acid.

In another aspect, disclosed are methods of preparing a conjugate, the method comprising: (a) reacting sortase with a nanobody portion, the nanobody portion including at least one nanobody having a sortase recognition site, to form a sortase-nanobody reagent, wherein the sortase-nanobody reagent comprises the nanobody portion, a nanobody linking moiety attached to the nanobody portion, the nanobody linking moiety including an amino acid sequence of LPXT (SEQ ID NO: 1), wherein X is any amino acid, and a sortase moiety attached to the nanobody linking moiety; (b) reacting the sortase-nanobody reagent with a reactive amine reagent, the reactive amine reagent comprising a first reactive group, to form a reactive nanobody reagent, the reactive nanobody reagent comprising the nanobody portion, the nanobody linking moiety, and a first reactive group attached to the nanobody linking moiety; and (c) coupling the reactive nanobody reagent with a reactive drug reagent, the reactive drug reagent comprising a drug and a second reactive group, to form a conjugate, the conjugate comprising the nanobody portion attached to the drug by a linker, the linker comprising the nanobody linking moiety.

In another aspect, disclosed are pharmaceutical compositions comprising one or more disclosed conjugates; and a pharmaceutically acceptable excipient.

In another aspect, disclosed are methods of treating a disease or a disorder in a subject in need thereof, the method comprising administering to the subject an effective amount of one or more conjugates as disclosed herein, optionally in combination with a pharmaceutically acceptable excipient.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1J show the design, synthesis, and in vitro characterization of an anti-albumin nanobody for site-selective conjugation of STING agonists.

FIG. 1A is a scheme depicting the concept of an albumin-hitchhiking nanobody-STING agonist conjugate for cancer immunotherapy. Anti-albumin nanobodies conjugated to STING agonists can bind to circulating albumin in situ, resulting in improved pharmacokinetics and increased biodistribution to tumor sites that stimulate antitumor innate and adaptive immune responses.

FIG. 1B illustrates the computational model of the anti-albumin nanobody (nAlb) binding at domain IIB of human serum albumin.

FIG. 1C shows isothermal calorimetry (ITC) traces (top) and binding isotherms (bottom) of nAlb binding to human and mouse serum albumin at pH 7.5.

FIG. 1D schematically depicts a reaction forgenerating molecularly homogeneous nAlb conjugates through site selective enzymatic ligation of an amine-PEG₃-azide followed by conjugation of agonist or dye cargo through copper-free click chemistry addition.

FIG. 1E shows the structure of diABZI STING agonist conjugated to a DBCO-PEG₁₁ handle for ligation to azide-functionalized nanobodies via click chemistry.

FIG. 1F is electrospray ionization mass spectrometry (ESI-MS) spectra demonstrating nanobody conjugate purity and molecular weight.

FIG. 1G shows sodium dodecyl sulfate polyacrylamide electrophoresis (SDS-PAGE) gel demonstrating nanobody conjugate purity and molecular weight.

FIG. 1H is a dose-response curve in the A549-Dual cell line (n=3) with estimated EC₅₀ values indicated in the legends.

FIG. 1I is a dose-response curve in the THP1-Dual IFN181-1 reporter cell line (n=3) with estimated EC₅₀ values indicated in the legends.

FIG. 1J shows qPCR analysis of gene expression in EMT6 murine breast cancer cells treated in vitro with 0.25 μM of free diABZI or nAlb-diABZI conjugate (n=3). P values determined by one-way ANOVA with post-hoc Tukey's correction for multiple comparisons; *P≤0.05, **P≤0.01, 184 ***P≤0.001, and ****P<0.0001 compared to PBS control. Data shown as mean±SEM.

FIGS. 2A-2K demonstrate that anti-albumin nanobodies harness albumin transport mechanisms to increase cargo delivery to tumor sites.

FIG. 2A schematically illustrates the proposed mechanism of albumin-hitchhiking, endocytosis, recycling, and degradation for intracellular STING agonist delivery.

FIG. 2B is a dose-response curve for nanobody-Cy5 conjugate surface binding and intracellular uptake at 37° C. and 4° C. measured by flow cytometry in EGFR⁻ (THP-1) and EGFR⁺ (A549) cells in vitro.

FIG. 2C shows bar graph analysis of nAlb-Cy5 uptake at 1 μM measured by flow cytometry in the presence or absence of receptor blocking antibodies (anti-GP60, 3 μM; anti-SPARC, 3 μM). P values determined by one-way ANOVA with post-hoc Tukey's correction for multiple comparisons; *P≤50.05, **P≤50.01, and ****P<0.0001 compared to PBS control.

FIG. 2D shows bar graph analysis of nAlb-Cy5 uptake at 1 μM measured by flow cytometry in serum-containing or serum-depleted media (n=3). ****P<0.0001 indicates a statistically significant difference between serum-containing or serum-depleted, as determined by Student's t-test.

FIG. 2E graphically shows the pharmacokinetics of free DBCO-Cy5 dye and indicated nanobody-Cy5 conjugates injected intravenously at 2 mg/kg in healthy female C57BL/6 mice (n=5). Elimination phase half-life and area under the curve (AUC) are indicated in legend.

FIG. 2F shows representative in vivo imaging system (IVIS) fluorescent images of excised tumors and major organs.

FIG. 2G shows bar graphs quantifying the average radiant efficiencies 24 h following intravenous administration of vehicle (PBS), DBCO-Cy5, nEGFR-Cy5, and nAlb-Cy5 at 2 mg/kg to female Balb/c 259 mice with orthotopic EMT6 breast tumors (n=5-8). P values determined by one-way ANOVA with post-hoc Tukey's correction for multiple comparisons; *P≤0.05, **P≤0.01, ***P≤0.001, and ****P<0.0001 compared to the tumor.

FIG. 2H shows heat map analysis of the ratio of tumor fluorescence to indicated organ fluorescence as measured by IVIS fluorescence imaging for free DBCO-Cy5 dye, nEGFR-Cy5, and nAlb-Cy5; **P≤0.01 and ****P<0.0001 indicate a statistically significant difference to DBCO-Cy5 by one-way ANOVA with post-hoc Tukey's correction for multiple comparisons.

FIG. 21 shows representative fluorescent microscopy images of tumor sections stained for DAPI (blue), CD45 (green), and CD31 (red) 24 h following administration of nAlb-Cy5 (yellow) alone or in combination with nAlb-diABZI (scale bar: 200 μm).

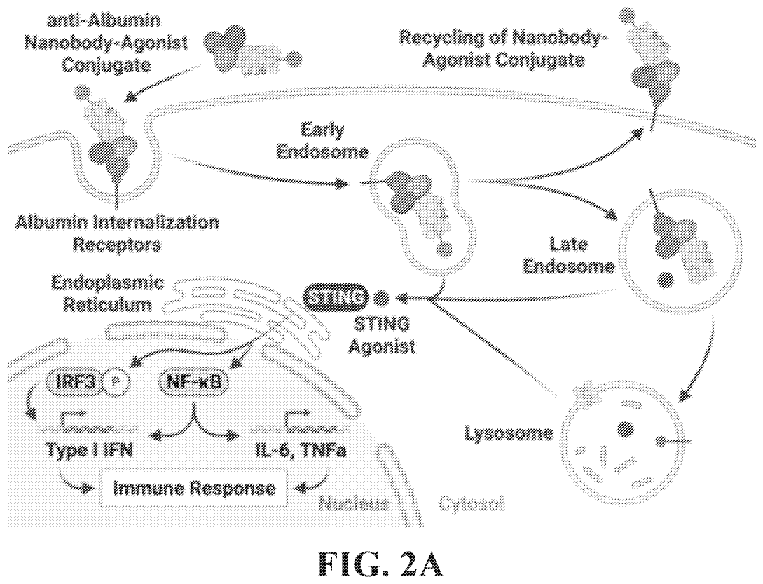

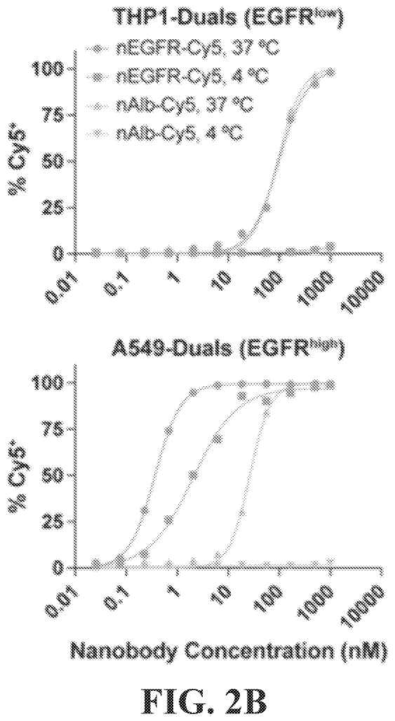

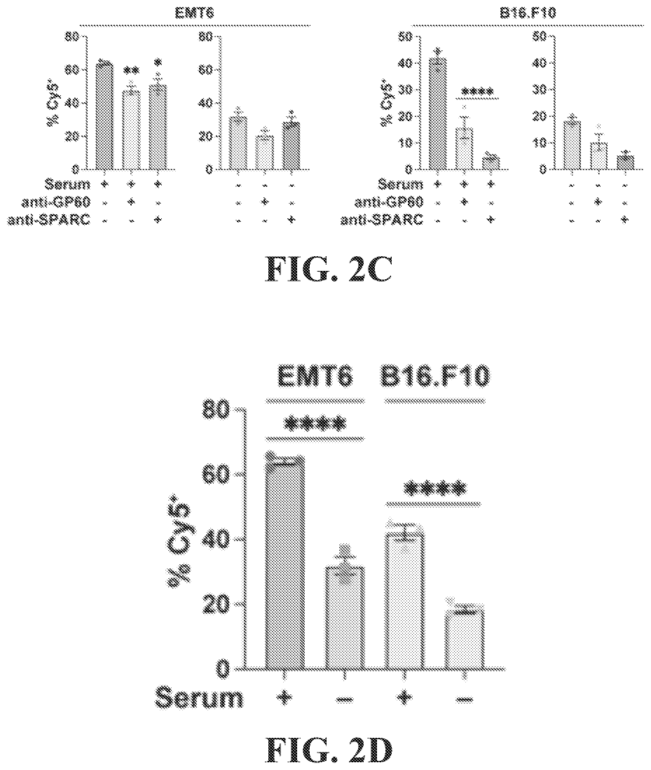

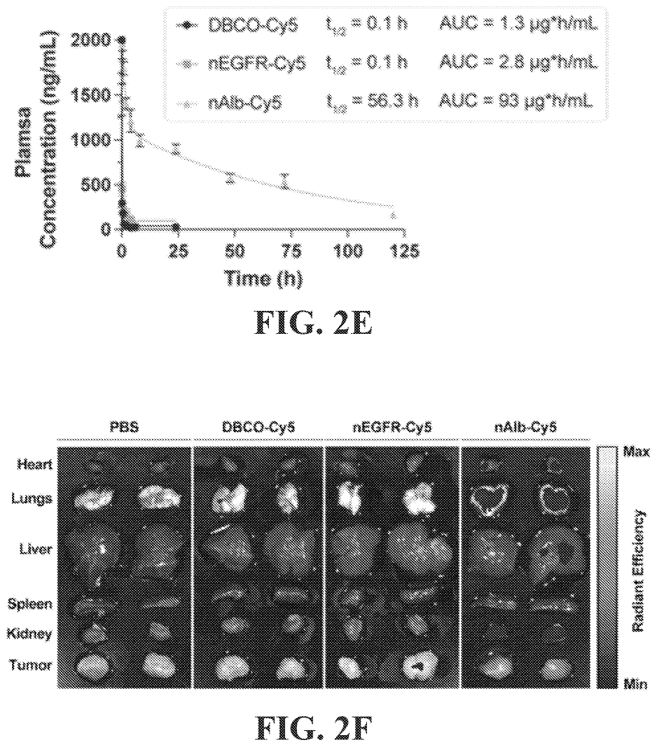

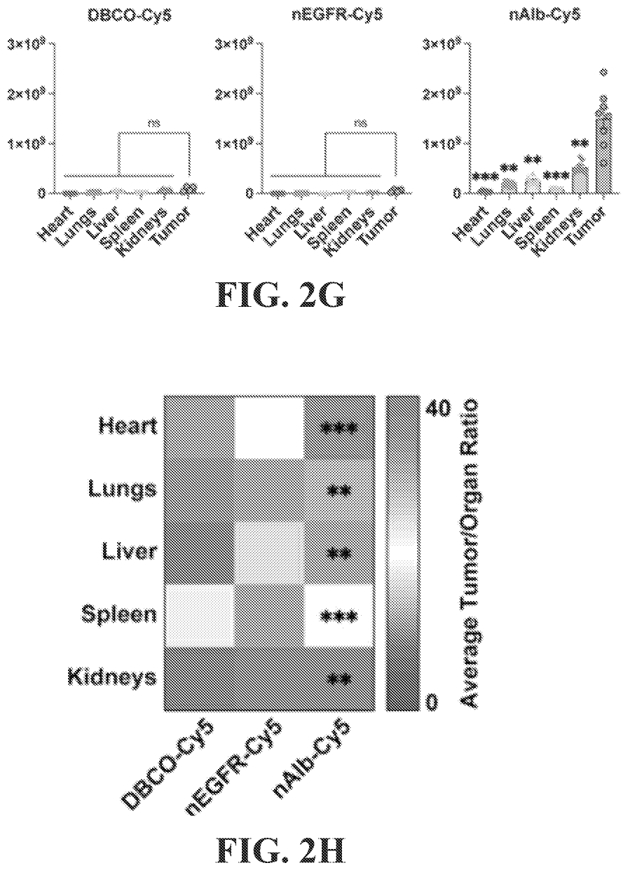

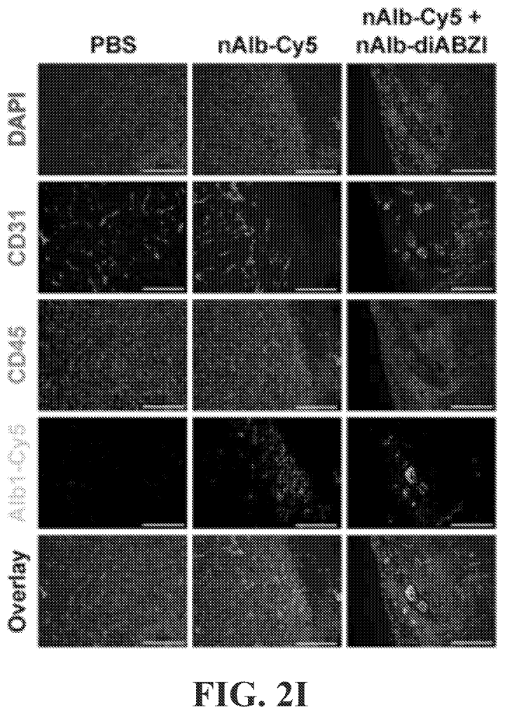

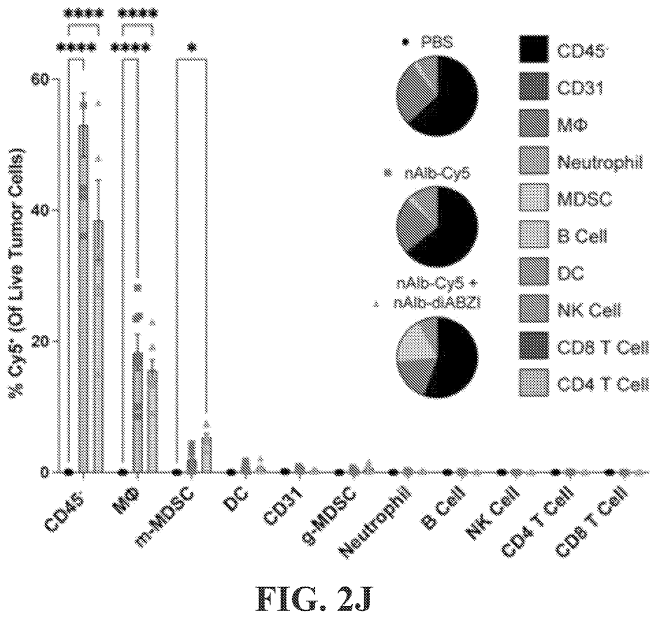

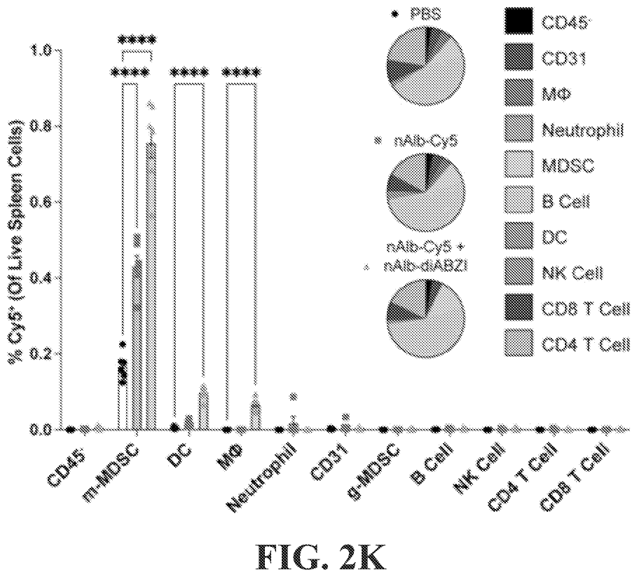

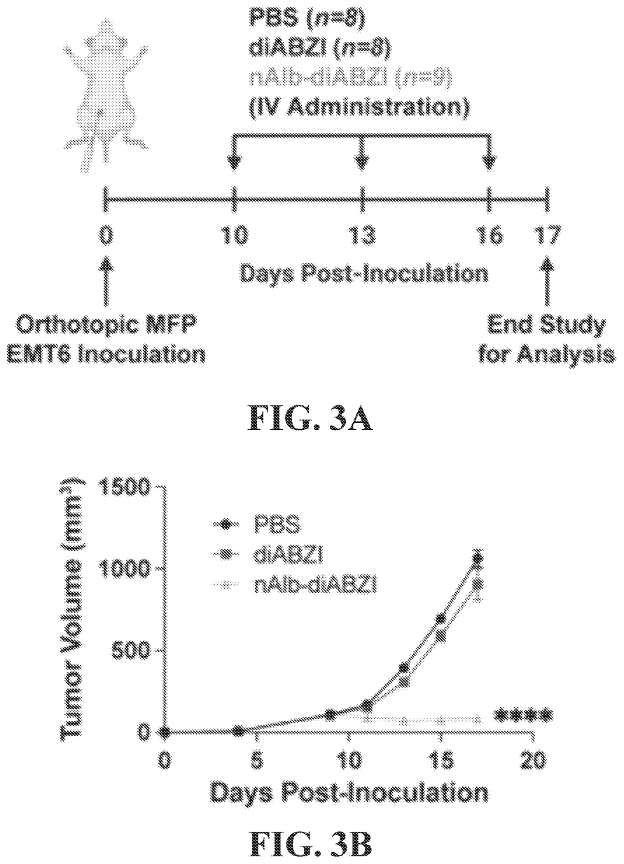

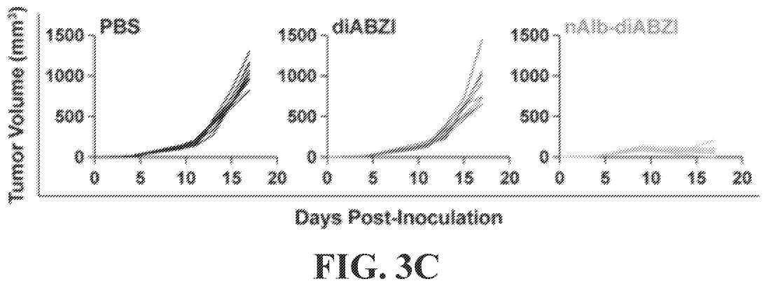

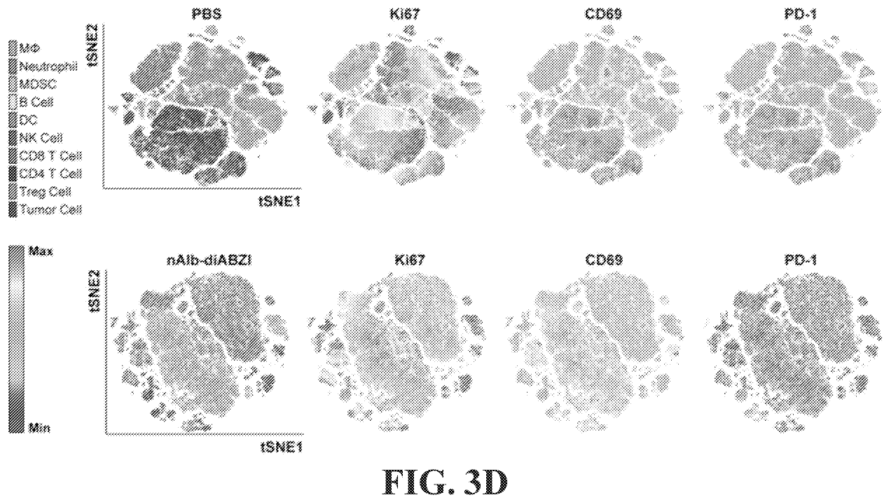

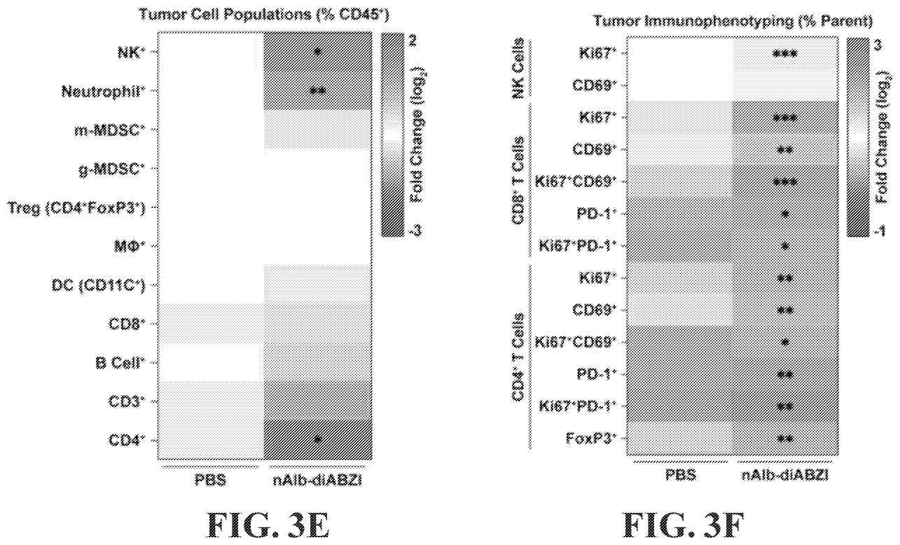

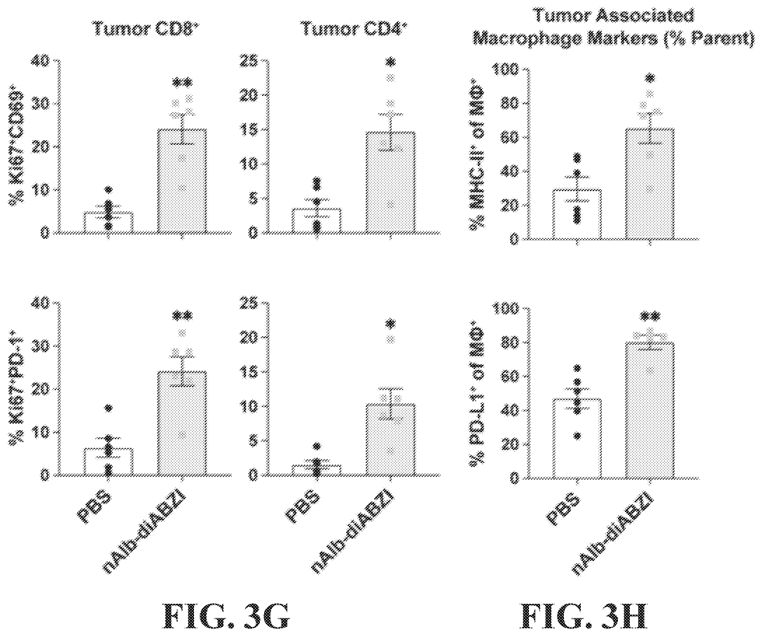

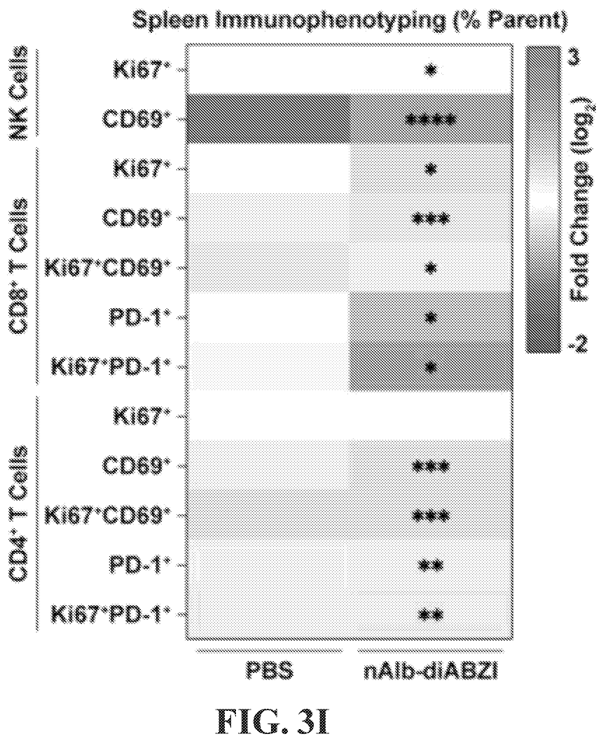

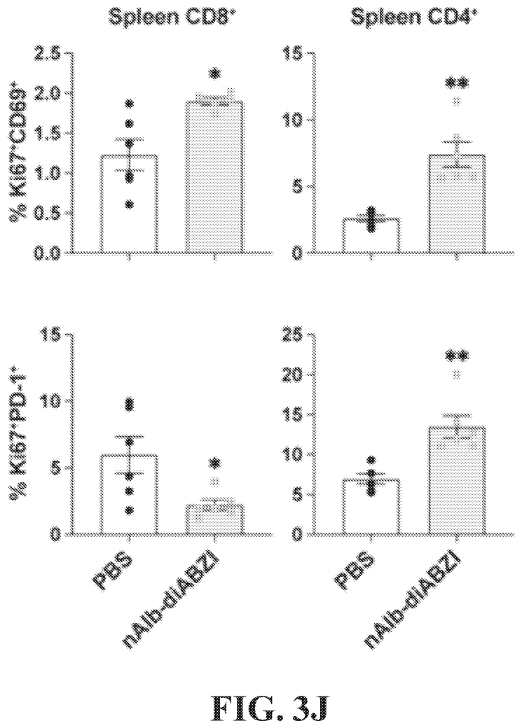

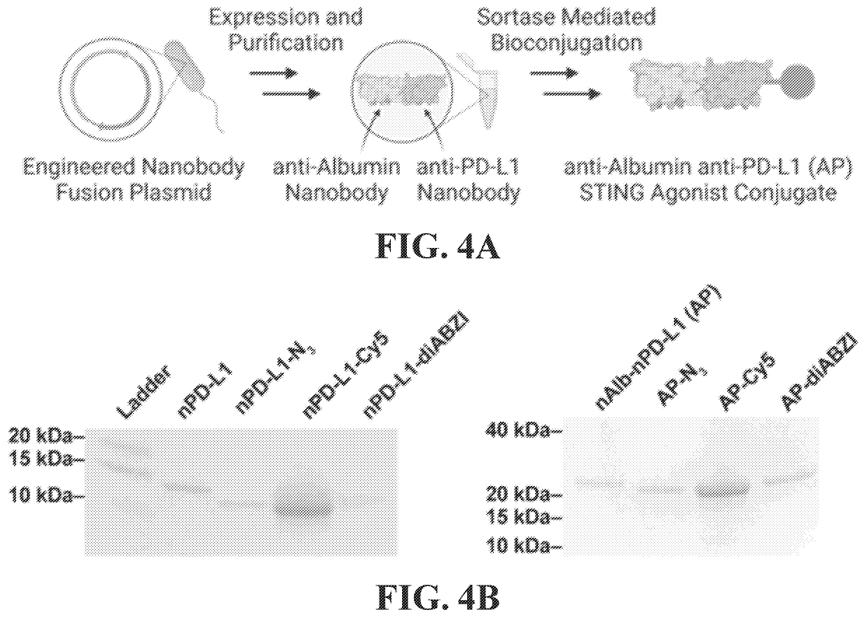

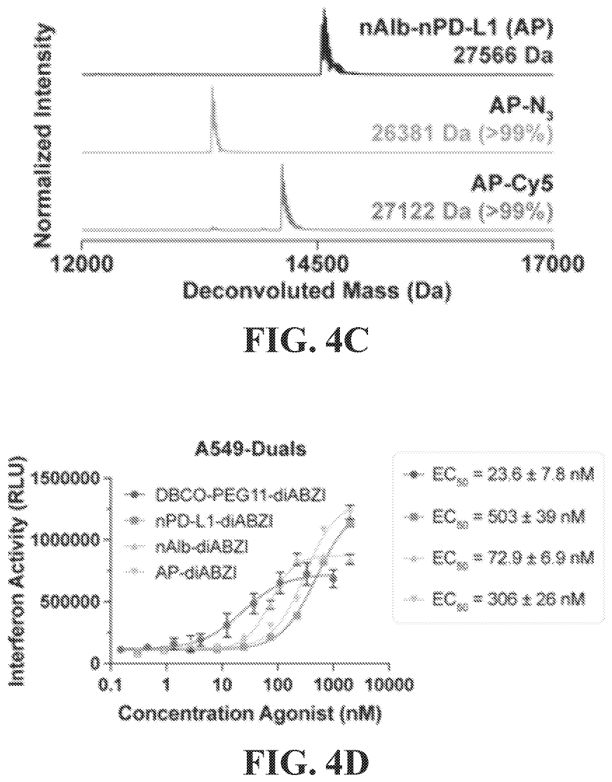

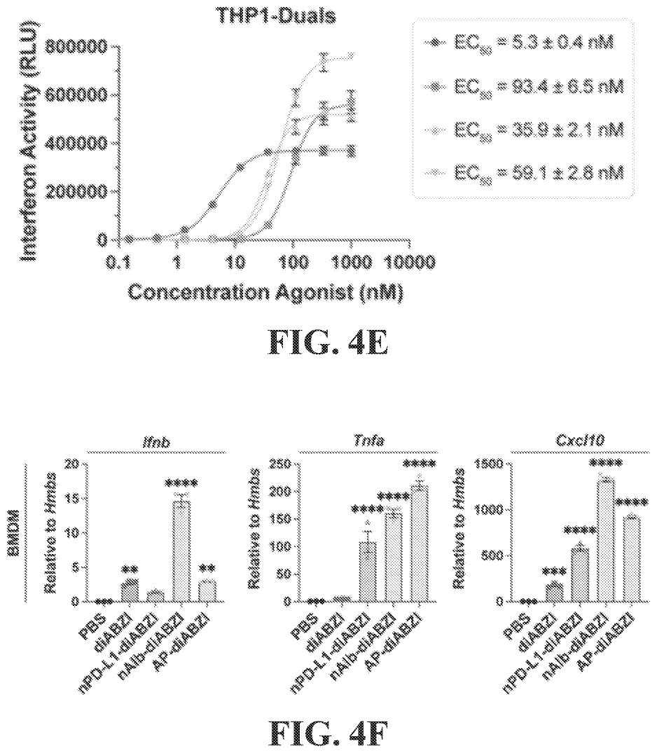

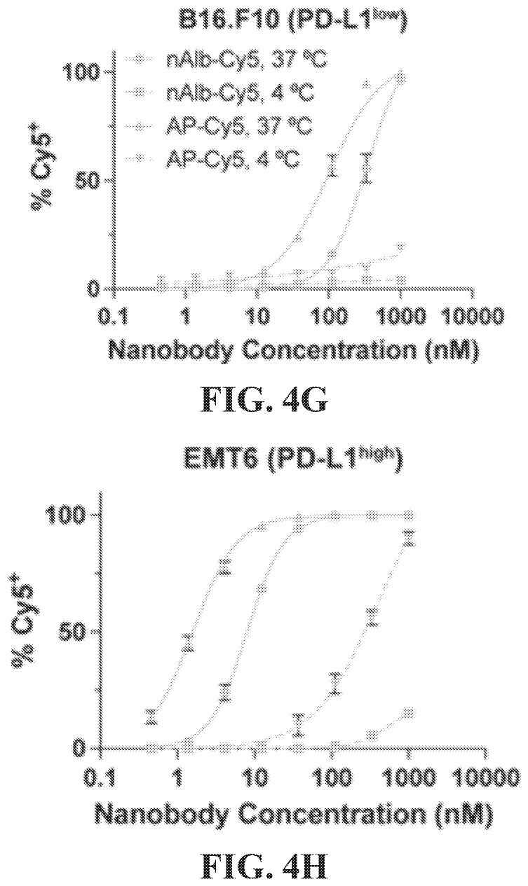

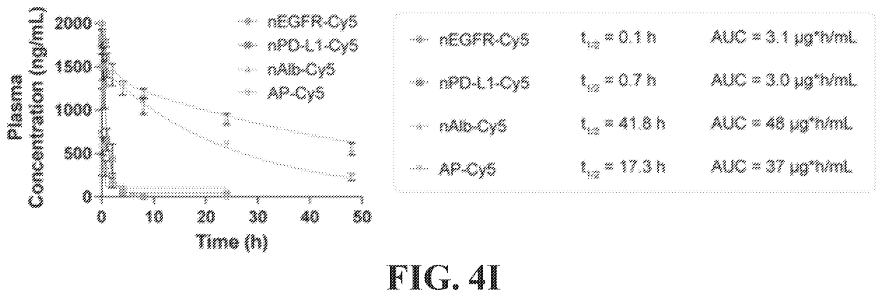

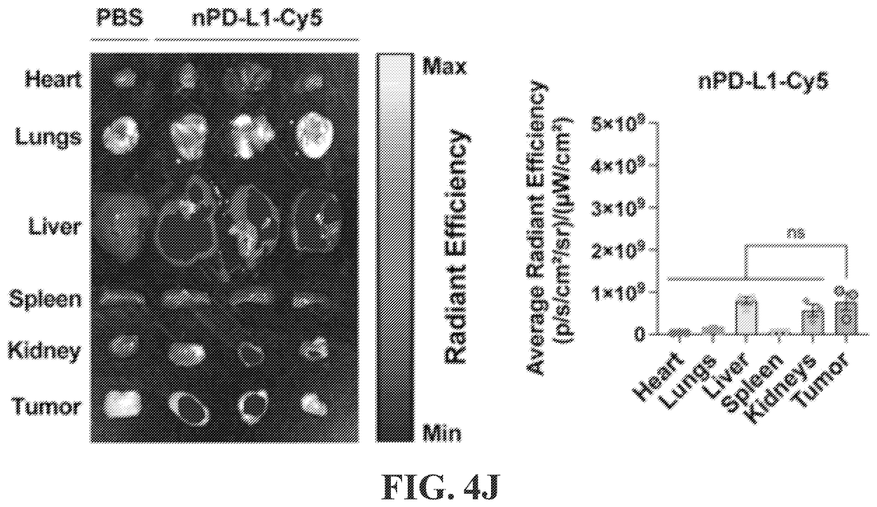

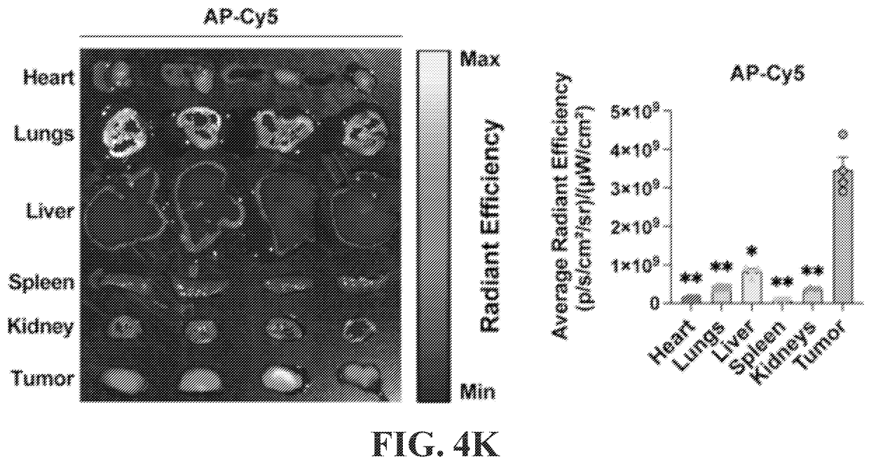

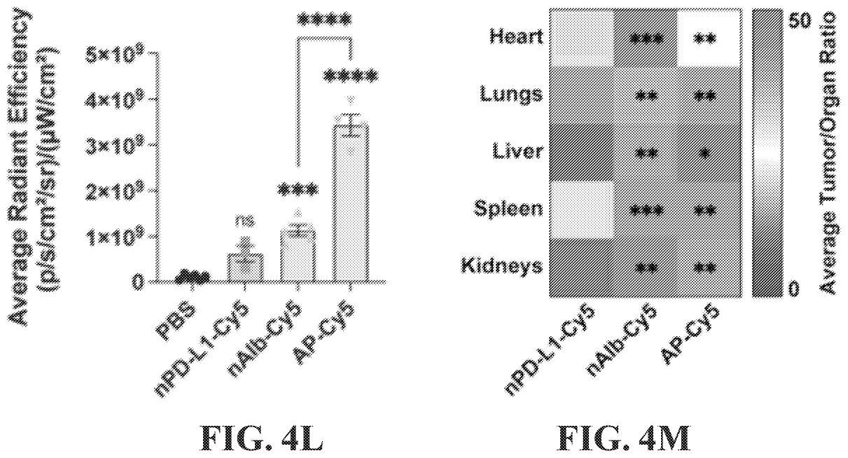

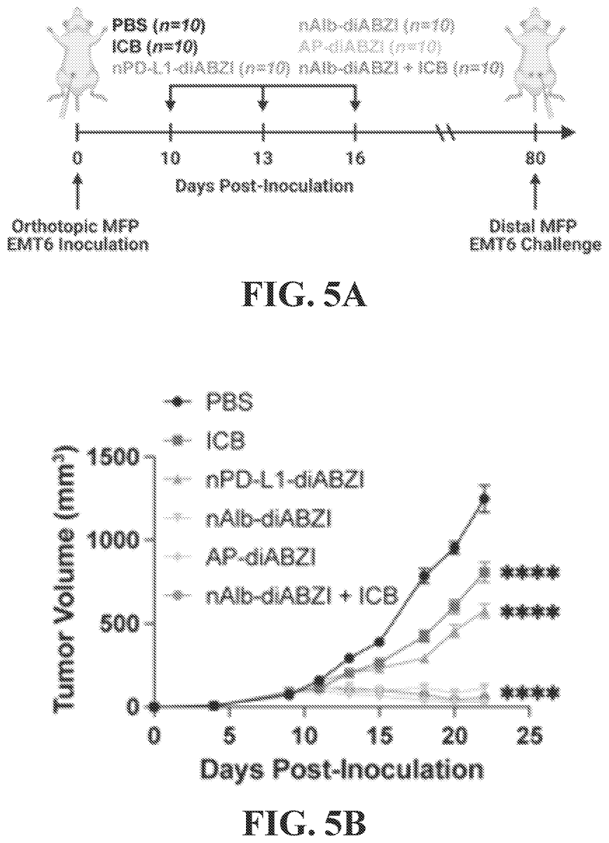

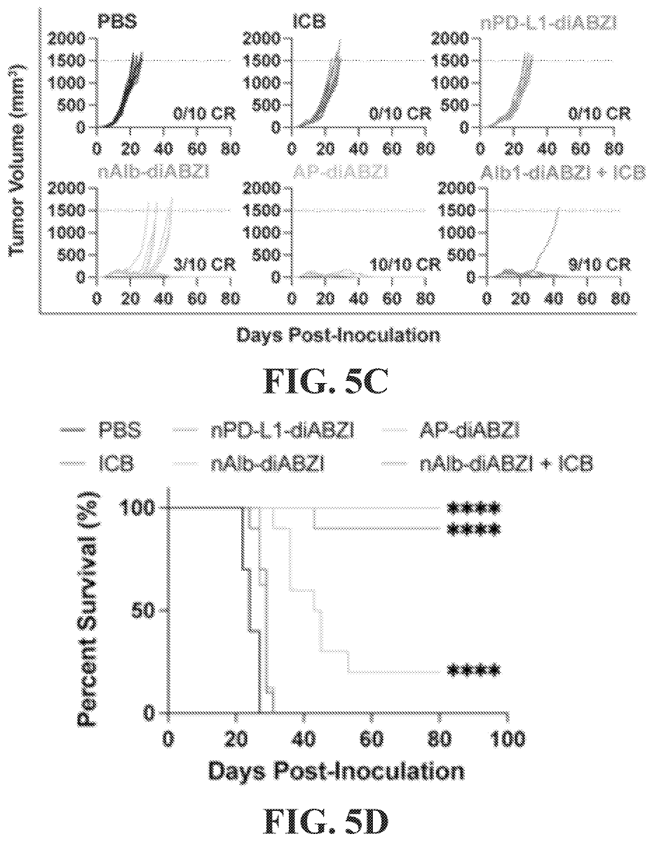

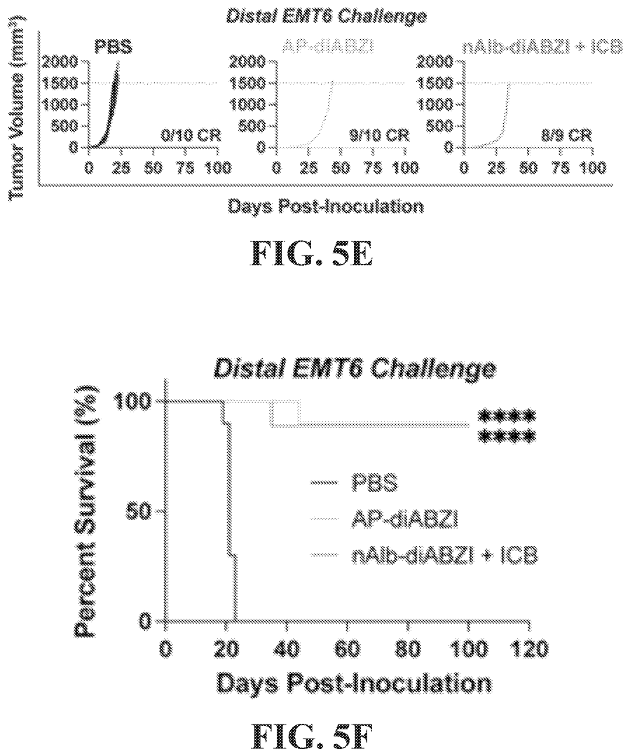

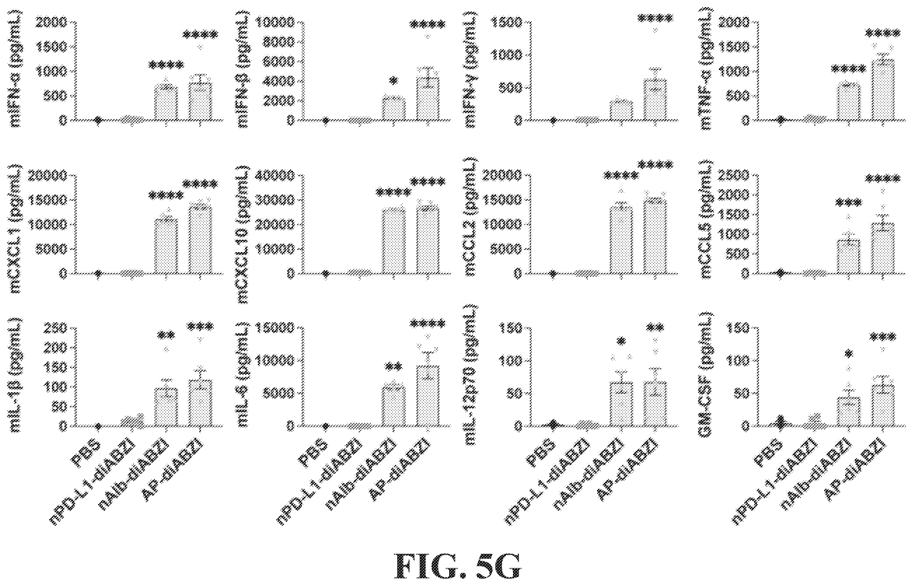

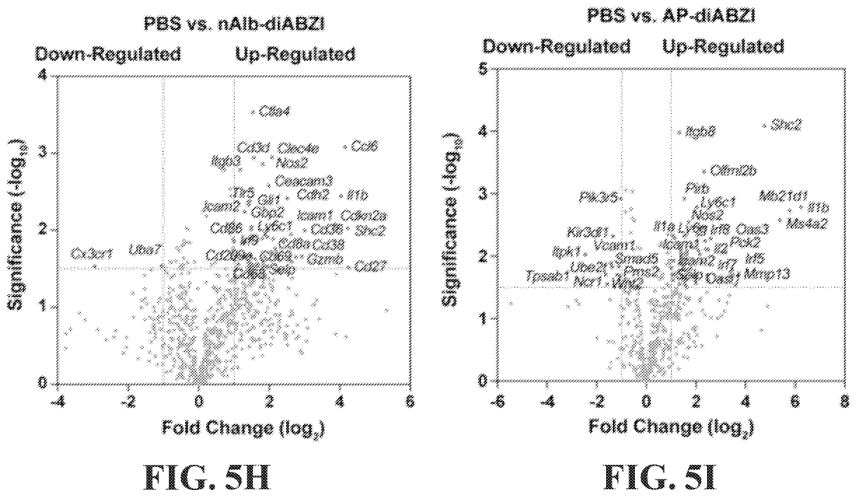

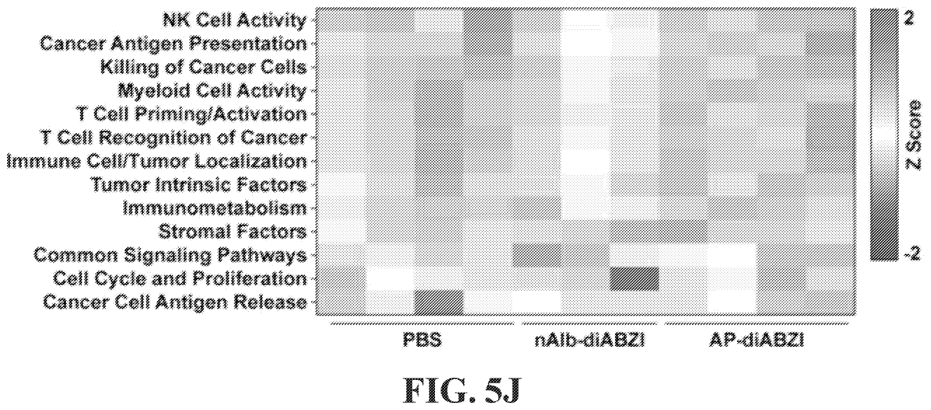

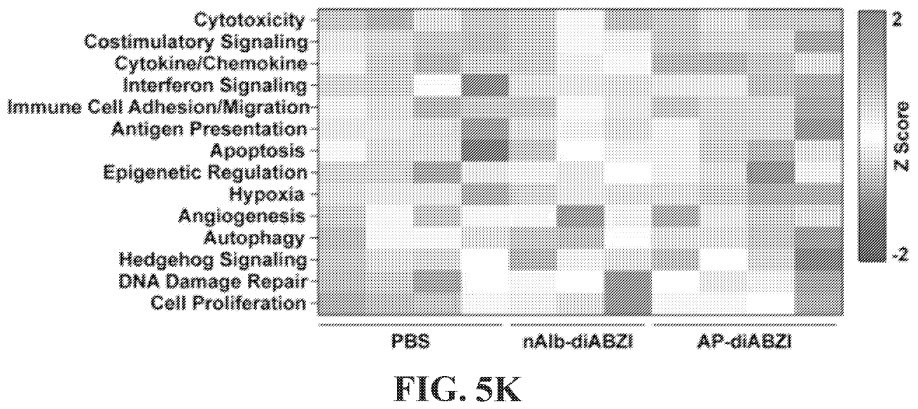

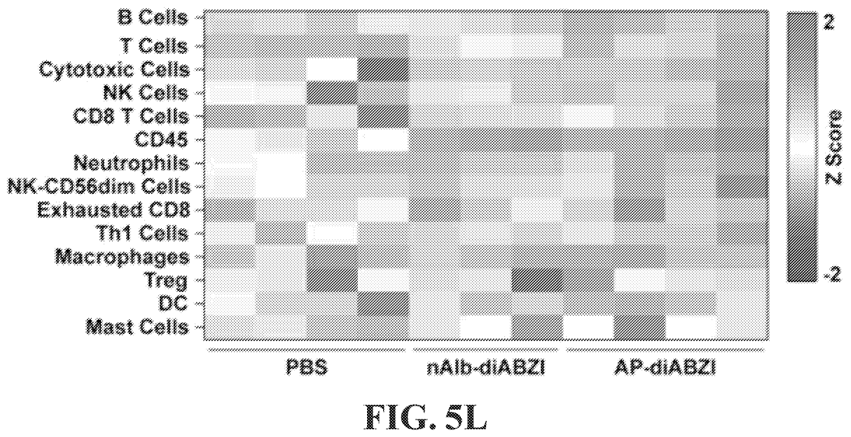

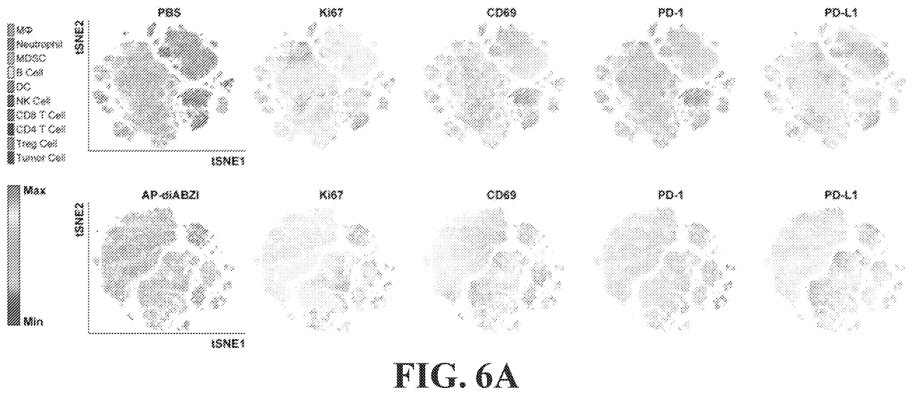

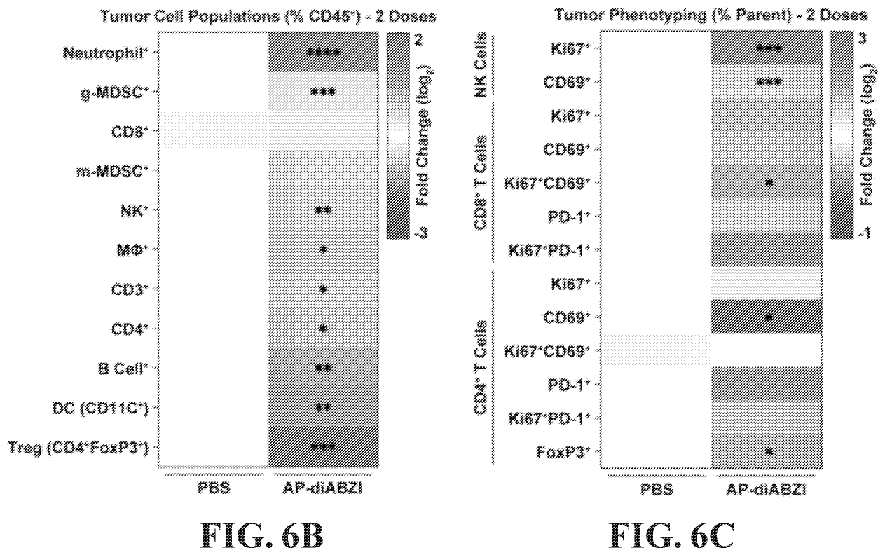

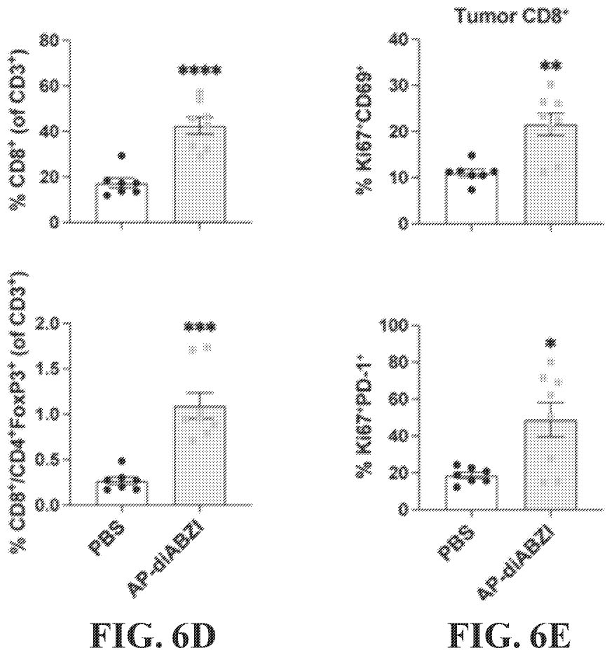

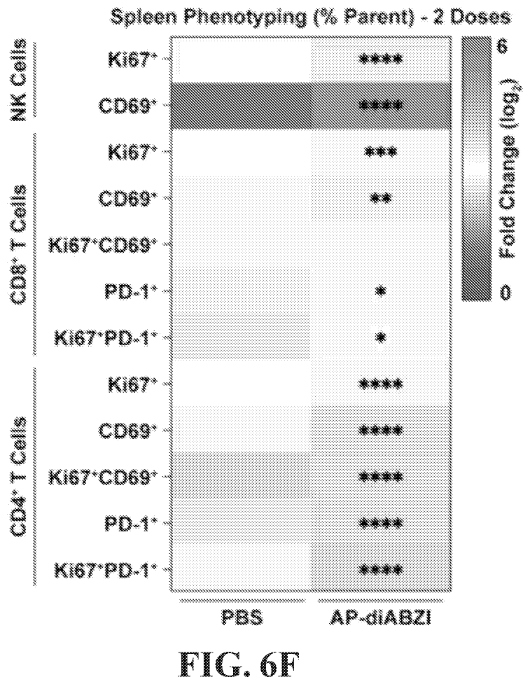

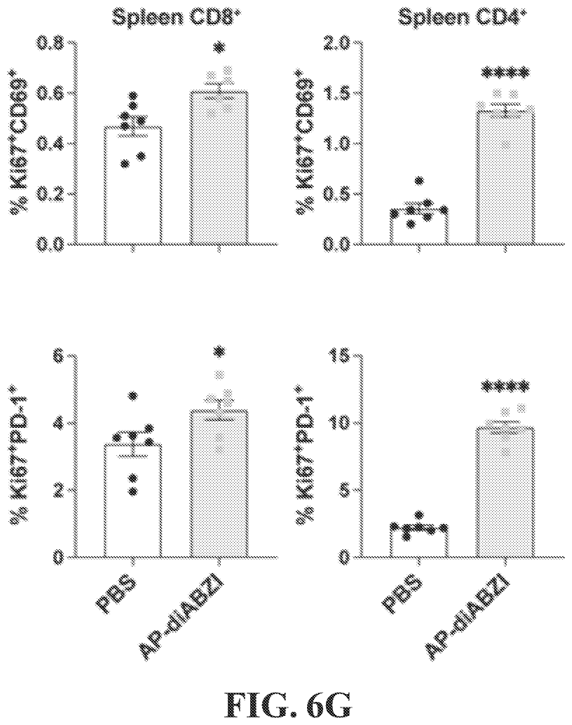

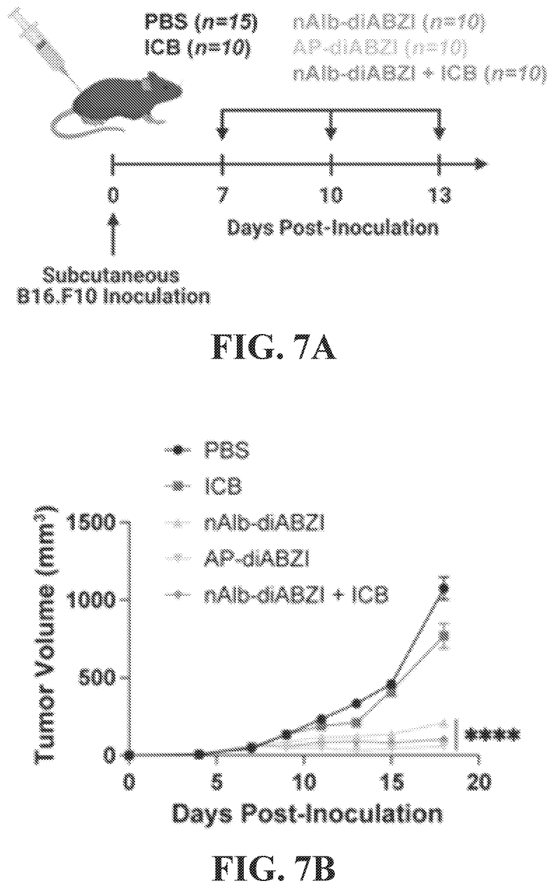

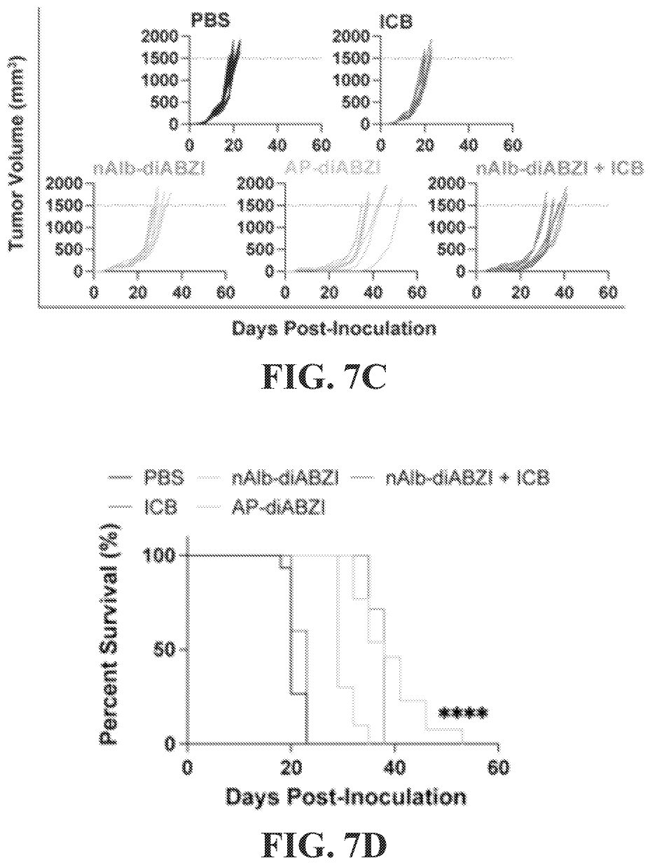

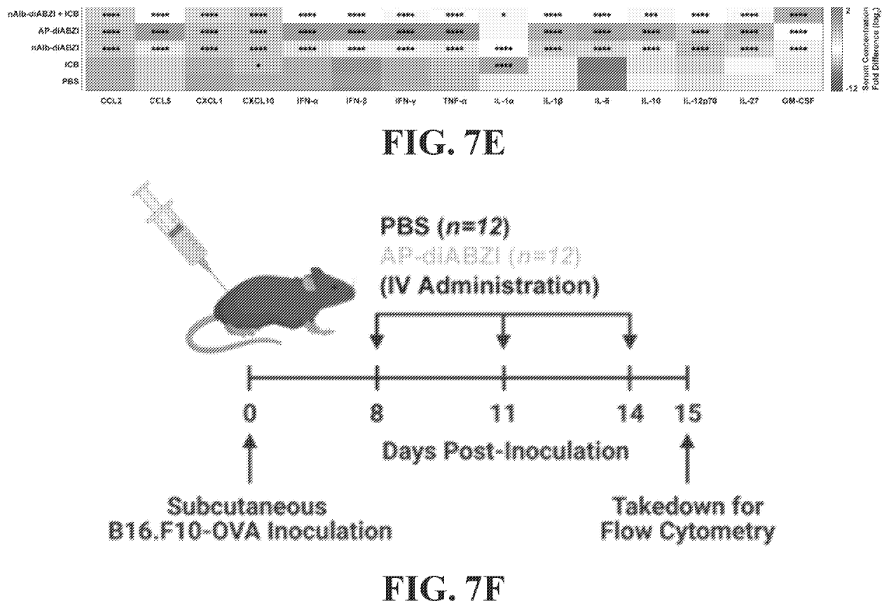

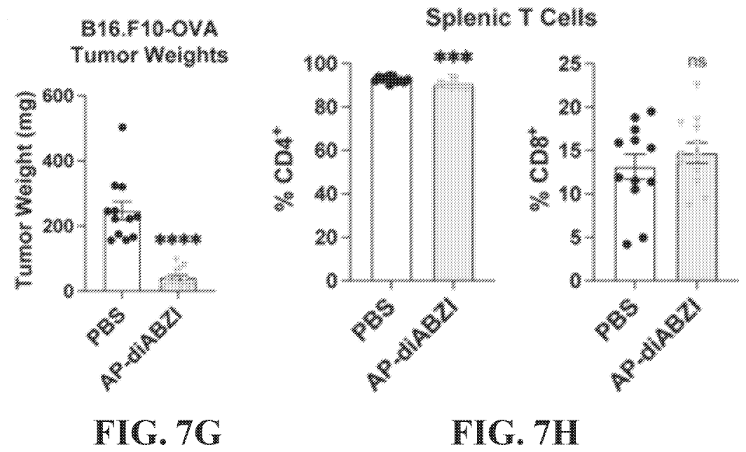

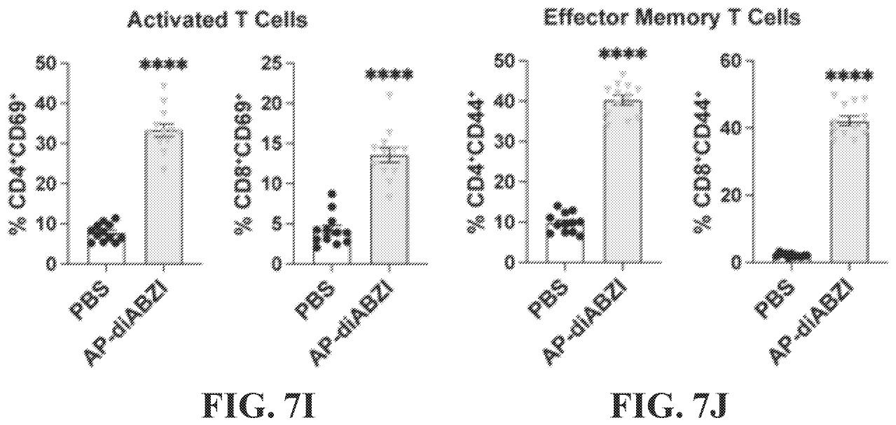

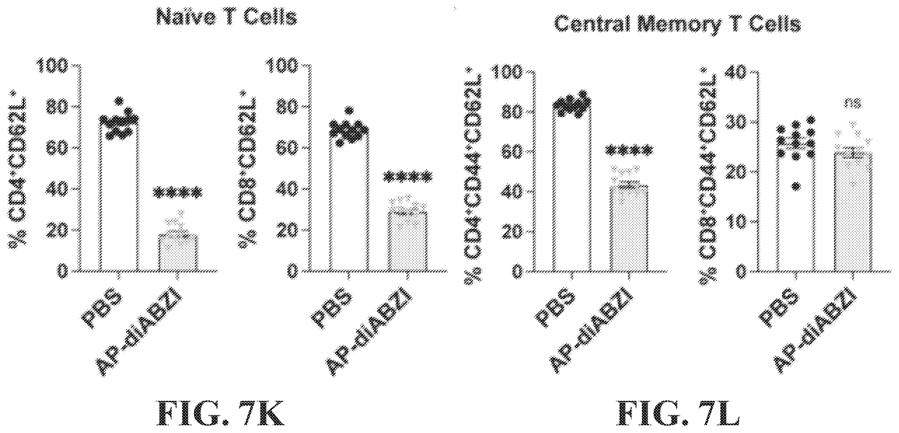

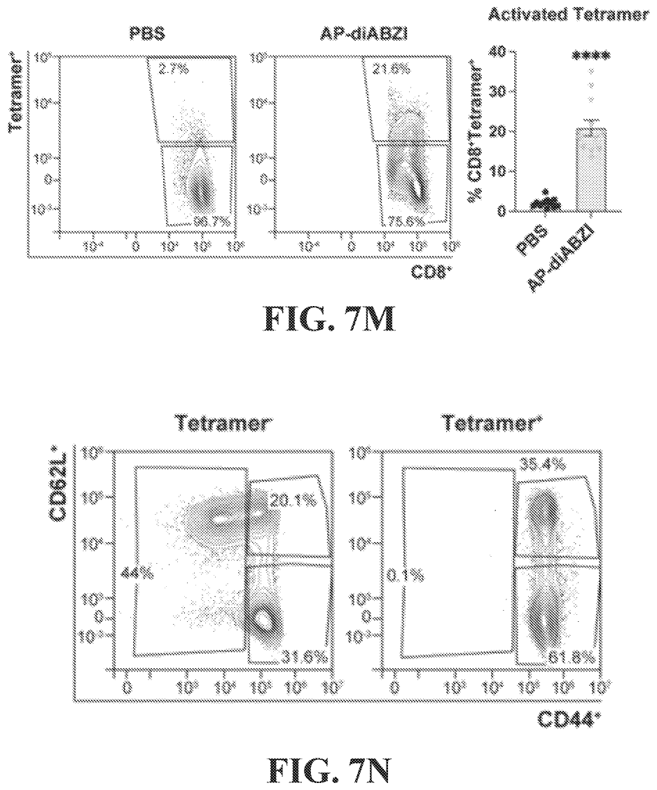

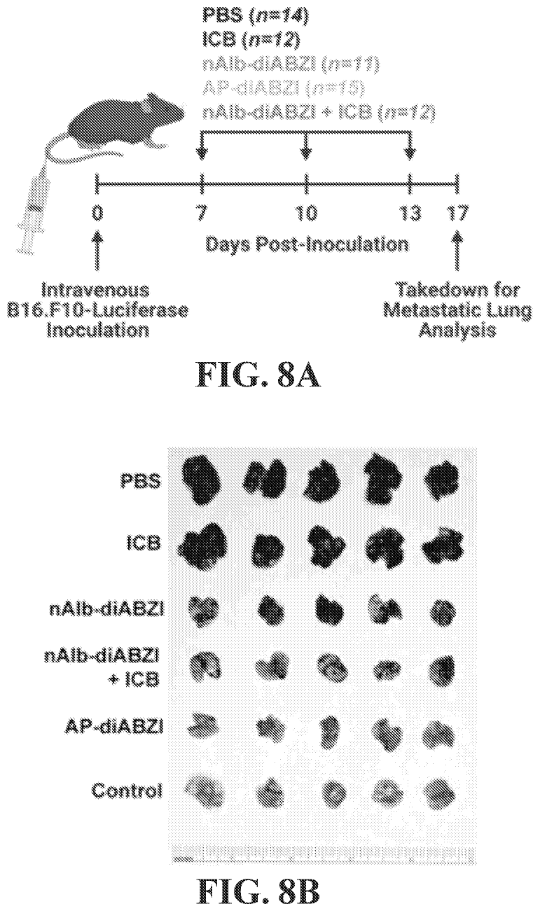

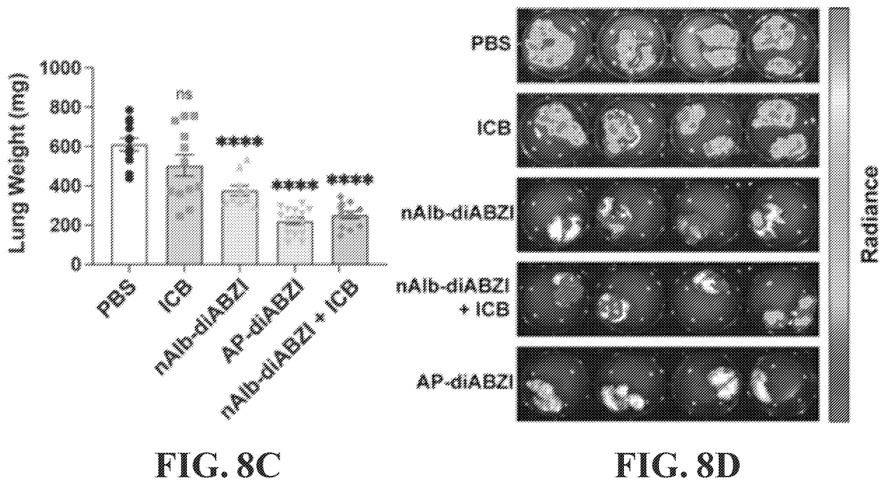

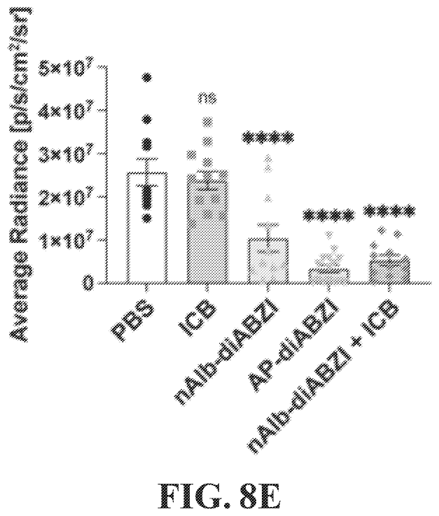

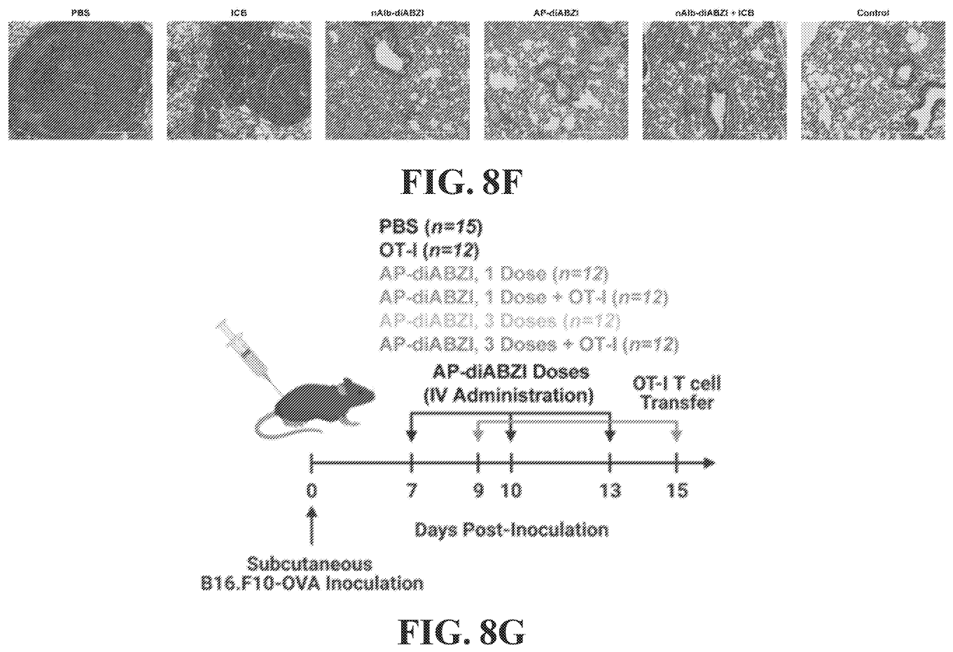

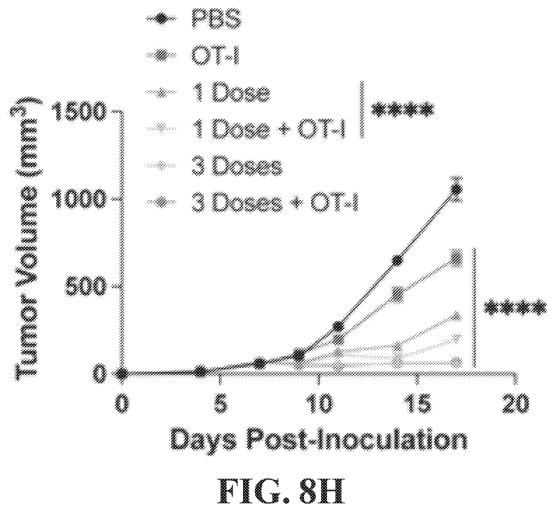

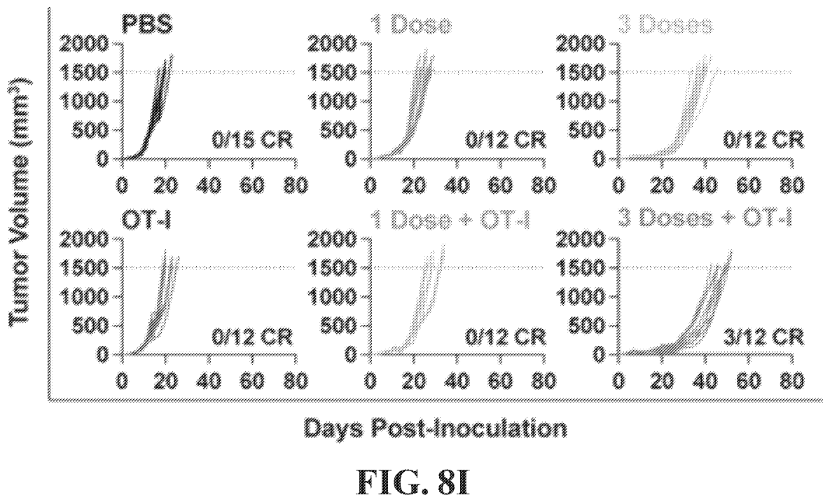

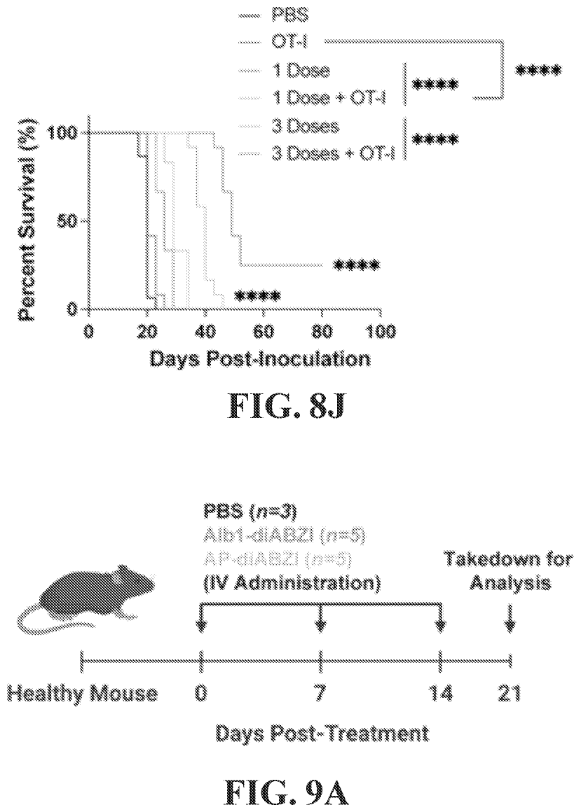

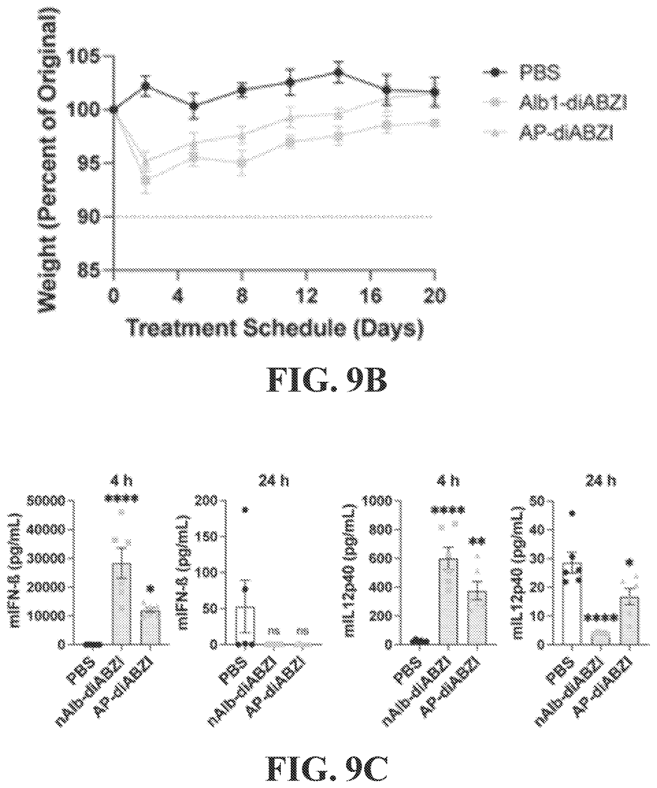

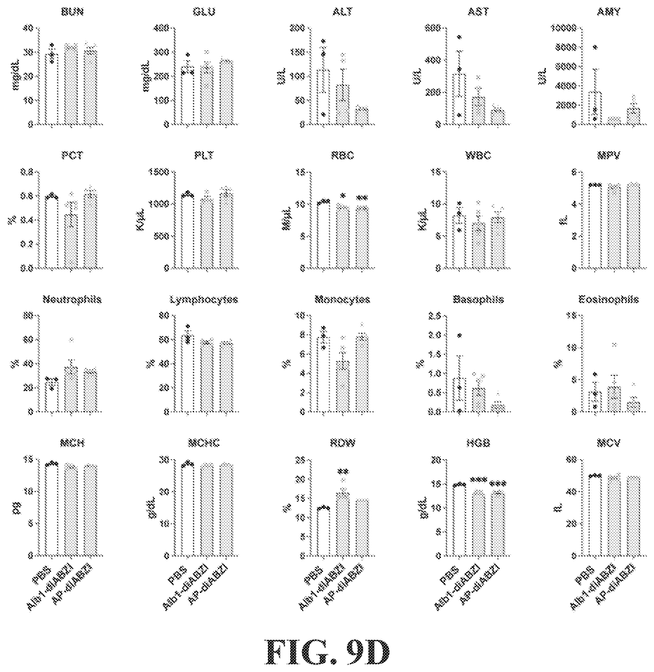

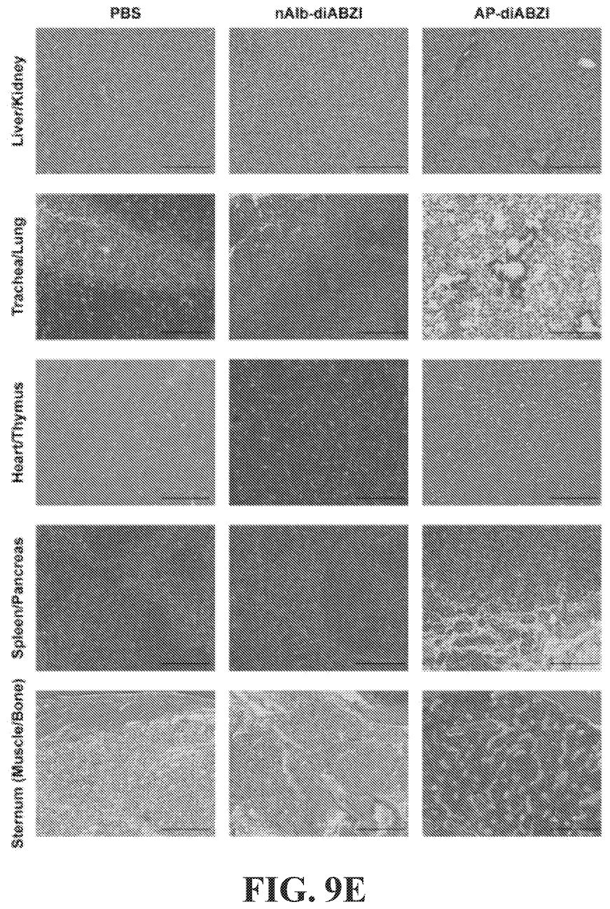

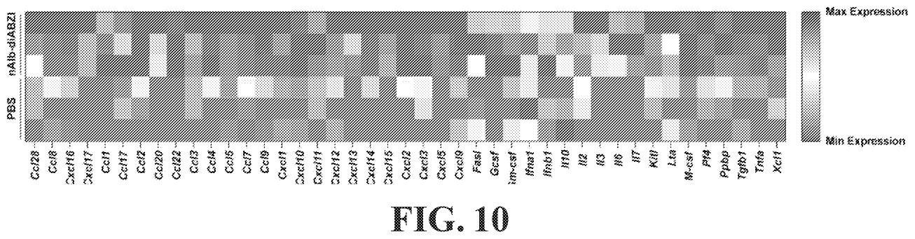

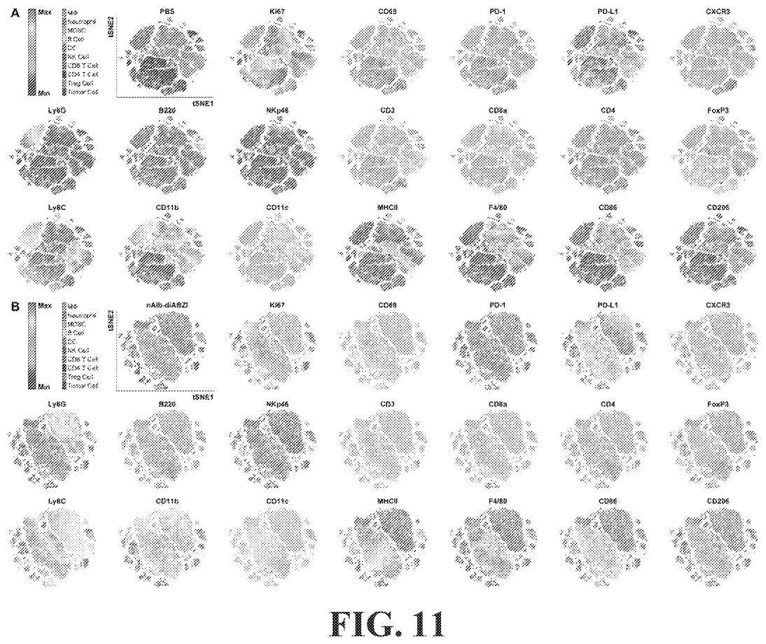

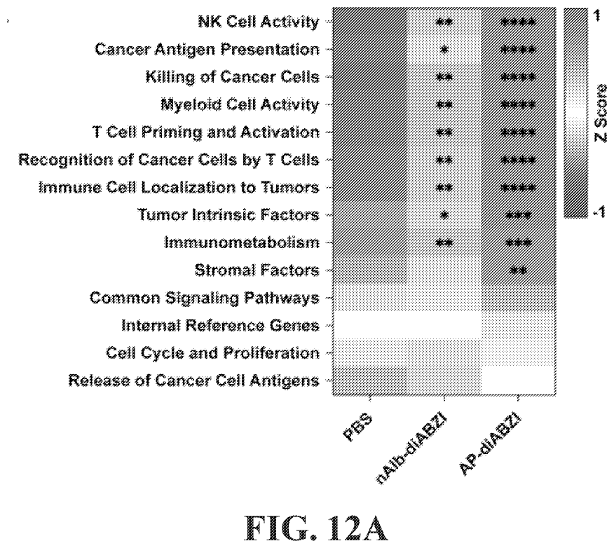

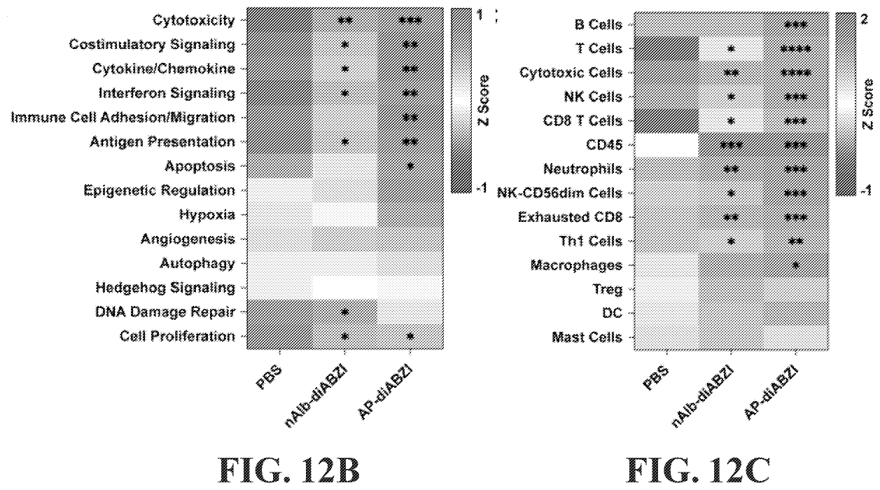

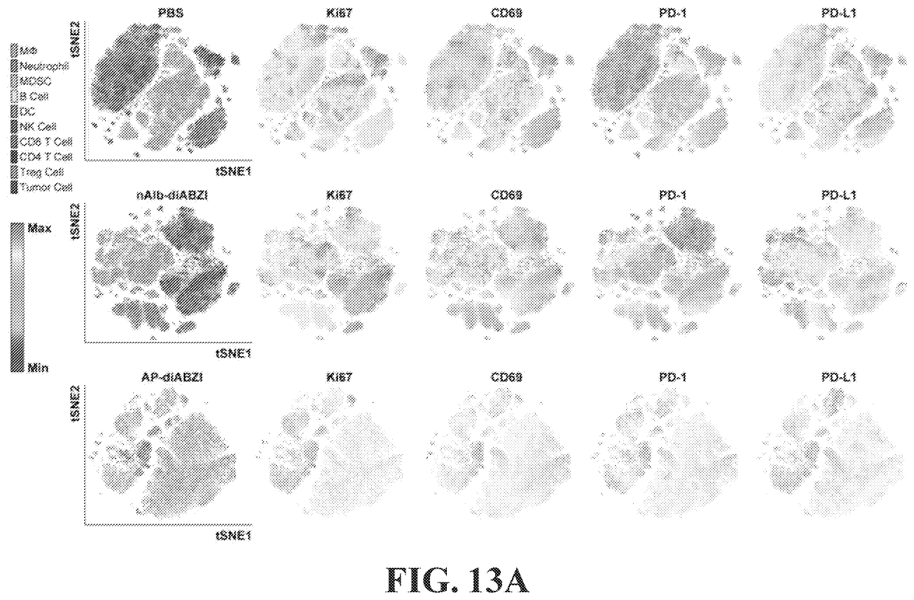

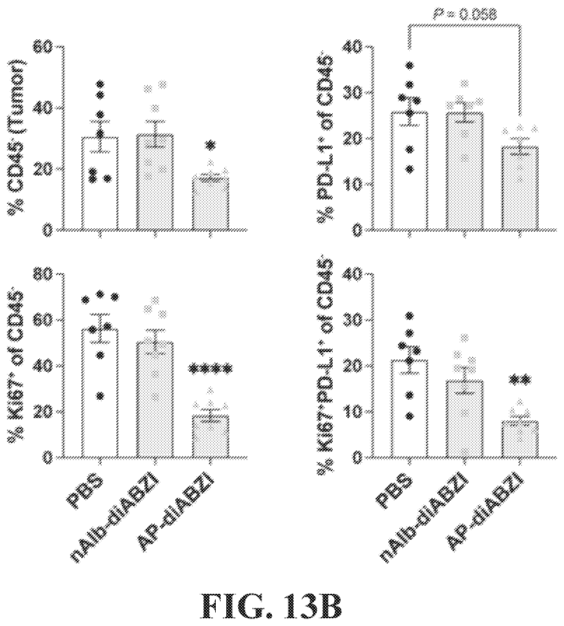

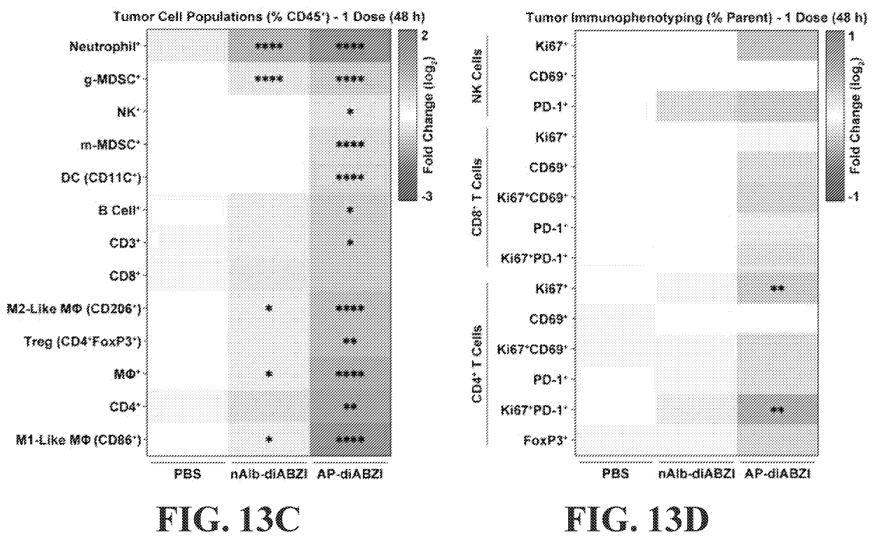

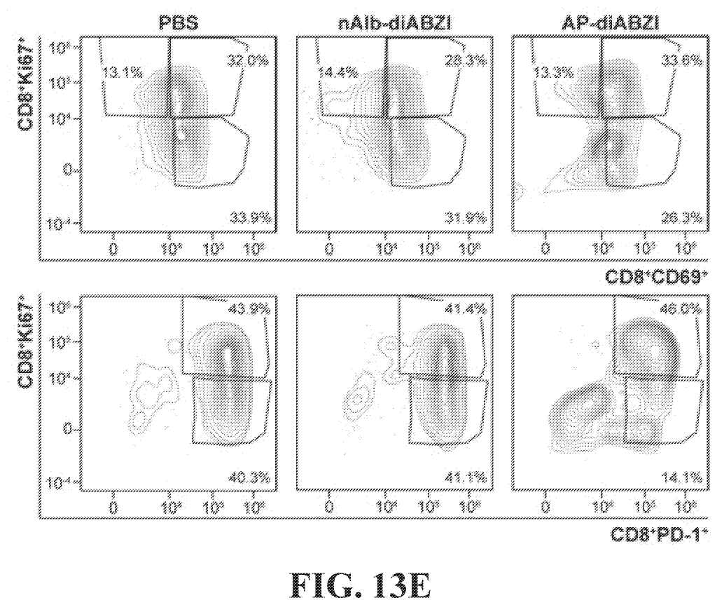

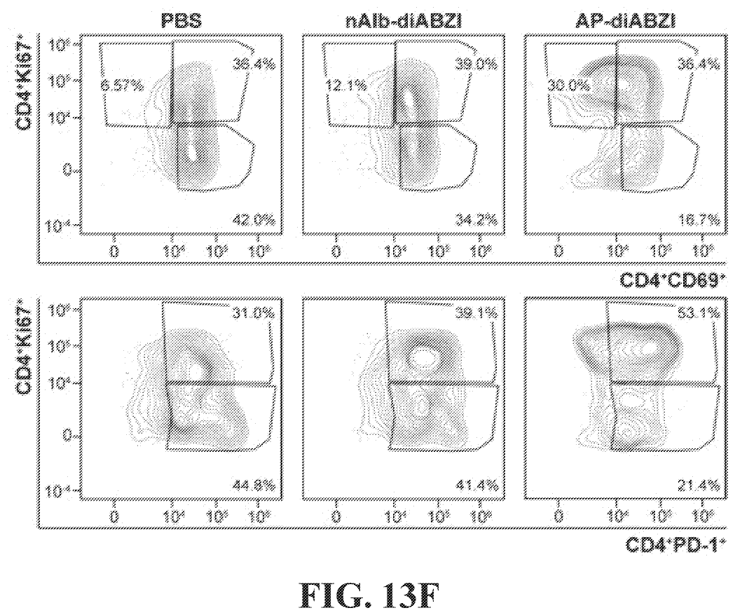

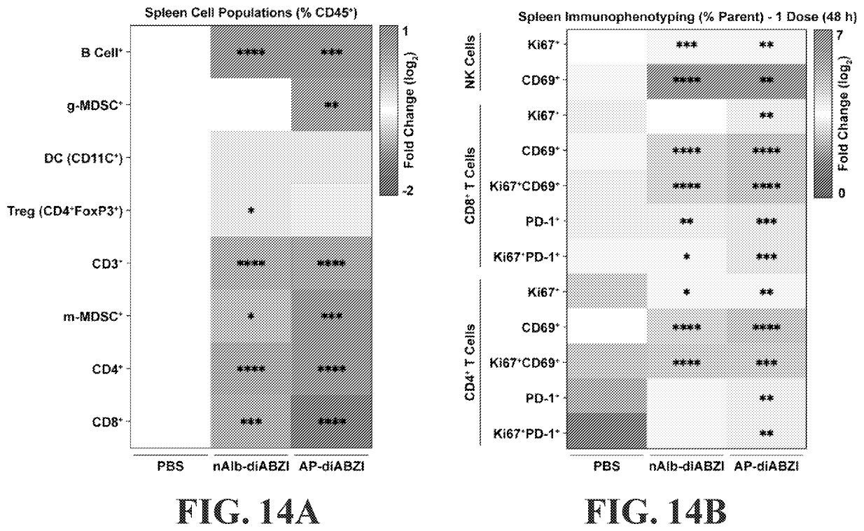

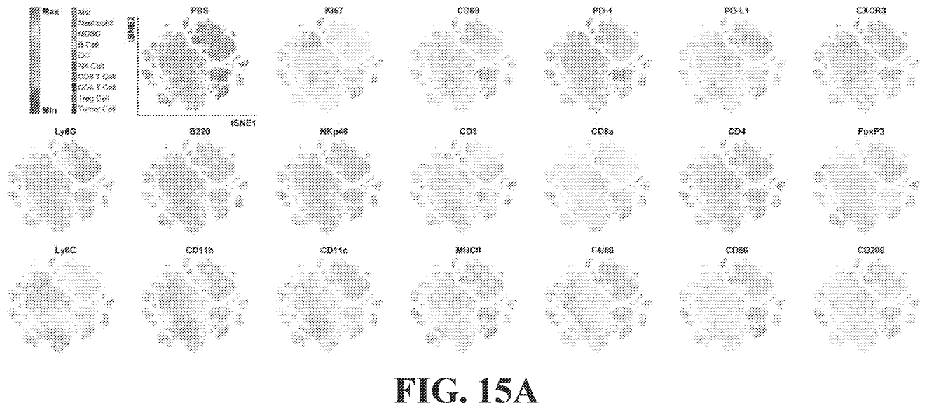

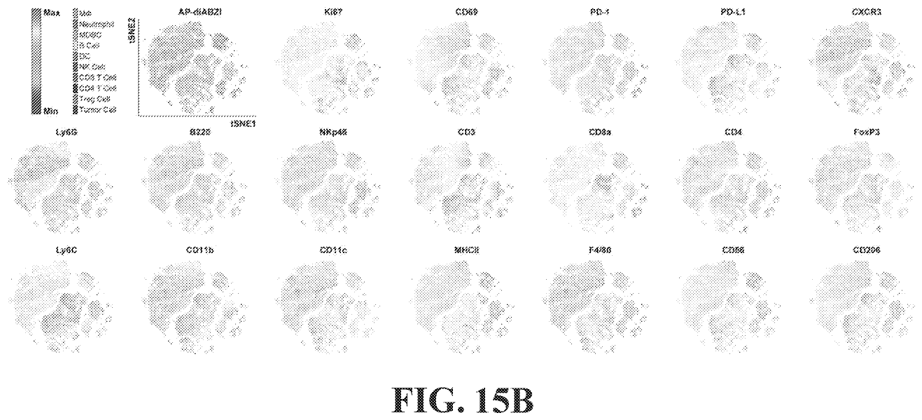

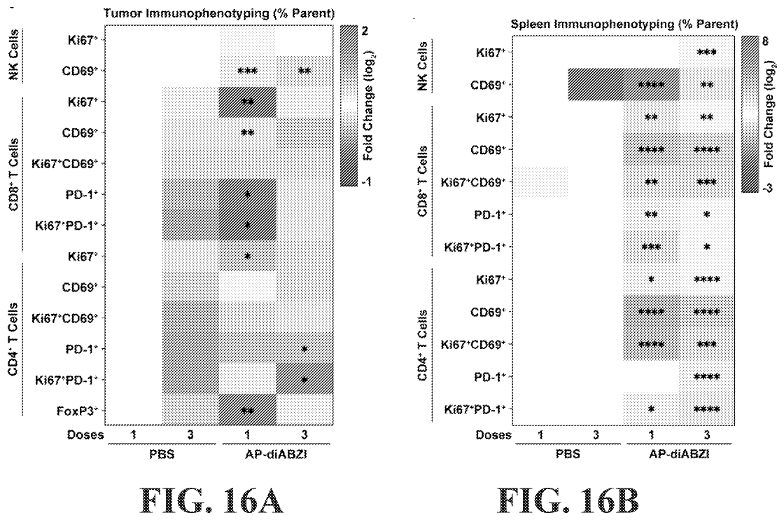

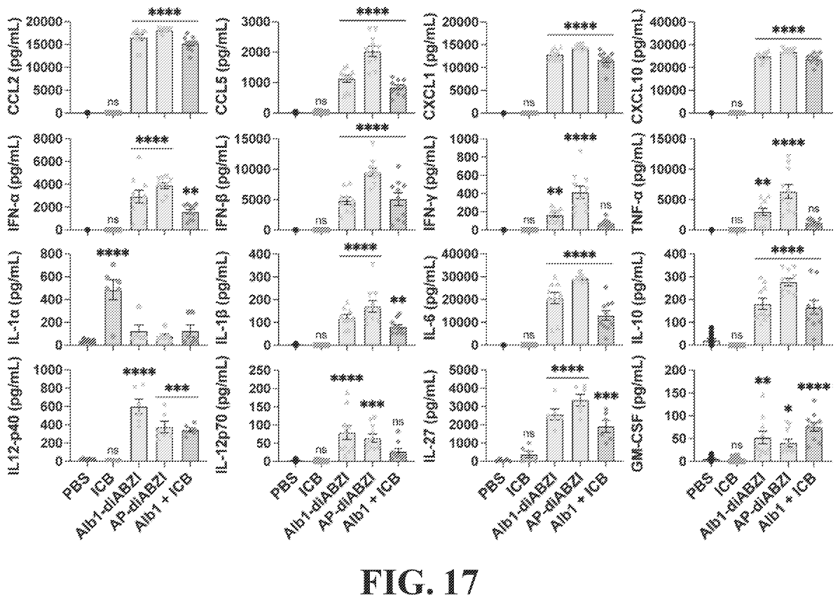

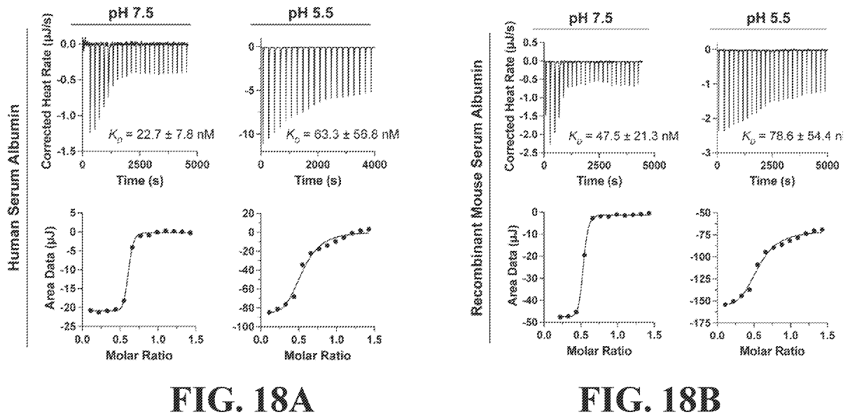

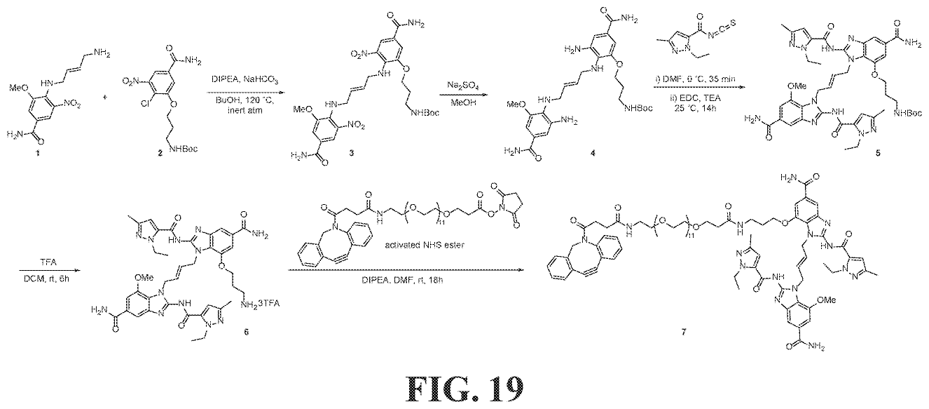

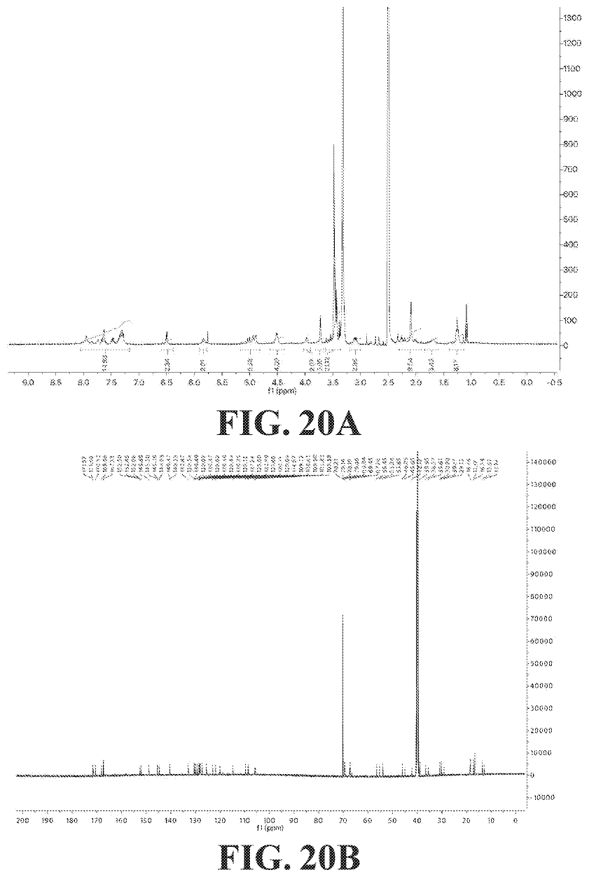

FIG. 2J shows flow cytometric analysis of nAlb-Cy5 uptake by indicated cell type as a percentage of total live cells in EMT6 tumors 24 h following administration of vehicle (PBS), nAlb-Cy5 alone, or nAlb-Cy5 co-administered with nAlb-diABZI; median fluorescent intensities (MFI) for each cell population are shown in FIGS. 22A-22B (n=7-8). Insets: percentage of indicated cell population in the tumor as measured by flow cytometry. DC: dendritic cell; Mφ: macrophage; MDSC: myeloid derived suppressor cell; NK: natural killer cell. P values determined by one-way ANOVA with post-hoc Tukey's correction for multiple comparisons; *P≤50.05, **P≤50.01, ***P≤50.001, and ****P<0.0001 compared to PBS control. Data shown as mean±SEM.

FIG. 2J shows flow cytometric analysis of nAlb-Cy5 uptake by indicated cell type as a percentage of total live cells in spleen 24 h following administration of vehicle (PBS), nAlb-Cy5 alone, or nAlb-Cy5 co-administered with nAlb-diABZI; median fluorescent intensities (MFI) for each cell population are shown in FIGS. 22A-22B (n=7-8). Insets: percentage of indicated cell population in the tumor as measured by flow cytometry. DC: dendritic cell; Mφ: macrophage; MDSC: myeloid derived suppressor cell; NK: natural killer cell. P values determined by one-way ANOVA with post-hoc Tukey's correction for multiple comparisons; *P≤50.05, **P≤50.01, ***P≤0.001, and ****P<0.0001 compared to PBS control. Data shown as mean±SEM.

FIGS. 3A-3J demonstrate that albumin-hitchhiking STING agonist inhibits breast tumor growth by shifting the immunocellular profile of the TME.

FIG. 3A schematically illustrates EMT6 tumor inoculations, treatment schedule, and study end point for gene expression and flow cytometry analysis.

FIG. 3B shows tumor growth curves of individual tumor growth curves for each mice with EMT6 tumors treated as indicated (n=8-9).

FIG. 3C shows spider plots of individual tumor growth curves for each mice with EMT6 tumors treated as indicated (n=8-9). SEM with P value determined by two-way ANOVA with post-hoc Tukey's correction for multiple comparisons; ****P<0.0001 on day 17 for all groups compared to PBS. FIG. 3(D-J) Flow cytometric analysis of breast tumors and spleen 24 h following final dose of nAlb333 diABZI.

FIG. 3D shows t-distributed stochastic neighbor embedding (tSNE) plots of live cells in EMT6 tumors colored by cell population with relative expression level of Ki67, CD69, and PD-1 as indicated on heat map. DC: dendritic cell; Mφ: macrophage; NK: natural killer cell; MDSC: myeloid-derived suppressor cell.

FIG. 3E is a heat map summarizing the fold change in the percentage of indicated cell population.

FIG. 3F is a heat map summarizing the fold change in the frequency of NK cells, CD8⁺ T cells, and CD4⁺ T cells expressing the indicated marker or marker combination in EMT6 breast tumors.

FIG. 3G shows bar graphs quantifying Ki67⁺CD69⁺ and Ki67⁺PD1⁺CD8⁺ and CD4⁺ T cells in EMT6 tumors following treatment with vehicle (PBS) or nAlb-diABZI.

FIG. 3H shows bar graphs quantifying the frequency of MHC-II+ and PD-L1+ macrophages in EMT-6 tumors following treatment with vehicle (PBS) or nAlb-diABZT.

FIG. 3I is a heat map summarizing fold change in the frequency of NK cells, CD8⁺ T cells, and CD4⁺ T cells expressing activation markers within splenic populations.

FIG. 3J shows bar graphs quantifying Ki67⁺CD69⁺, Ki67⁺PD1⁺CD8⁺, and CD4⁺ T cells in spleens. *P≤0.05, **P≤50.01, ***P≤0.001, and ****P<0.0001 indicate a statistically significant difference between PBS and nAlb-diABZI treated groups as determined by Student's t-test, n=6 per group. All results are the mean f SEM.

FIGS. 4A-4M illustrate the design, synthesis, and testing of bivalent nanobody-STING agonist conjugate for albumin-hitchhiking and targeting of PD-L1.

FIG. 4A schematically illustrates the cloning, expression, and bioconjugation of small molecule cargo to generate the AP-diABZI conjugate.

FIG. 4B shows a sodium dodecyl sulfate-polyacrylamide gel electrophoresis (SDS-PAGE) image confirming the purity and molecular weight of the nPD-L1 and AP conjugates.

FIG. 4C shows an electrospray ionization mass spectrometry (ESI-MS) spectrum confirming the purity and molecular weight of the nPD-L1 and AP conjugates.

FIG. 4D shows dose-response curves for indicated nanobody-diABZI conjugate in A549-Dual (n=3) with estimated EC50 values indicated in the legends.

FIG. 4E shows dose-response curves for indicated nanobody-diABZI conjugate in THP1-Dual IFN-I reporter cell lines (n=3) with estimated EC50 values indicated in the legends.

FIG. 4F shows bar graphs providing qPCR analysis of genes associated with STING activation in bone marrow derived macrophages (BMDMs) in response to 4 h treatment with indicated agonist at 0.25 μM (n=5-6). P values determined by one-way ANOVA with post-hoc Tukey's correction for multiple comparisons; *P≤0.05, **P≤0.01, ***P≤0.001, and ****P<0.0001 compared to PBS control.

FIG. 4G is a dose response curve for nAlb-Cy5 and AP-Cy5 conjugate surface binding and intracellular uptake at 37° C. and 4° C. measured by flow cytometry in B16.F10 cells (n=2-3).

FIG. 4H is a dose response curve for nAlb-Cy5 and AP-Cy5 conjugate surface binding and intracellular uptake at 37° C. and 4° C. measured by flow cytometry in EMT6 cells (n=3).

FIG. 4I graphically shows pharmacokinetics of indicated nanobody-Cy5 conjugate in healthy Balb/c female mice (n=5). Elimination phase half-life and area under the curve (AUC) are indicated in legend.

FIG. 4J shows representative IVIS fluorescent images of excised tumors and major organs (left) and quantification of average radiant efficiencies (right) of tumors and major organs 48 h after administration of nPD-L1-Cy5 mice with EMT6 breast tumors (n=3-4). P values determined by one-way ANOVA with post-hoc Tukey's correction for multiple comparisons; *P≤0.05, **P≤50.01, ***P≤0.001, and ****P<0.0001 compared to the tumor group.

FIG. 4K shows representative IVIS fluorescent images of excised tumors and major organs (left) and quantification of average radiant efficiencies (right) of tumors and major organs 48 h after administration of AP-Cy5 in mice with EMT6 breast tumors (n=3-4). P values determined by one-way ANOVA with post-hoc Tukey's correction for multiple comparisons; *P≤0.05, **P≤0.01, ***P≤0.001, and ****P<0.0001 compared to the tumor group.

FIG. 4L graphically compares Cy5 radiant efficiencies in tumor tissue 48 h following administration of indicated nanobody-Cy5 conjugate. P values determined by one-way ANOVA with post-hoc Tukey's correction for multiple comparisons; *P≤0.05, **P≤0.01, ***P≤0.001, and ****P<0.0001 compared to the PBS control, as well as between nAlb-Cy5 and AP-Cy5.

FIG. 4M shows heat map analysis of the ratio of tumor fluorescence to indicated organ fluorescence as measured by IVIS fluorescence imaging for indicated nanobody-Cy5 conjugate. *P≤0.05, **P≤0.01, and ***P≤0.001 indicate a statistically significant difference compared to nPD-L1-Cy5 by one-way ANOVA with post-hoc Tukey's correction for multiple comparisons. All results are the mean±SEM.

FIGS. 5A-5L demonstrate that systemic administration of AP-diABZI conjugates enhance antitumor immune and therapeutic responses in EMT6 breast cancer model.

FIG. 5A schematically illustrates EMT6 tumor inoculation and treatment schedule (n=10). Anti-PD-L1 IgG (ICB) was injected I.P. at 100 μg and all nanobodies were injected 1.V. at 1.25 μg of diABZI per injection.

FIG. 5B shows tumor growth curves of mice treated with indicated formulation ****P<0.0001 on day 22 for all groups compared to PBS.

FIG. 5C shows spider plots of individual tumor growth curves using 1500 mm³ tumor volume as endpoint criteria with P value was determined by log-rank test; ****P<0.0001 compared to PBS control.

FIG. 5D shows Kaplan-Meier survival plots for mice with EMT6 tumors treated as indicated. CR=complete responder; SEM with P value determined by two-way ANOVA with post-hoc Tukey's correction for multiple comparisons.

FIG. 5E shows spider plots of individual tumor growth curves.

FIG. 5F shows Kaplan-Meier survival curves of mice challenged or re-challenged (complete responders after first treatment regimen) with EMT6 cells (n=9-10).

FIG. 5G shows serum cytokine/chemokine concentrations 4 h post-injection with indicated nanobody-diABZI conjugate (n=6-10). P values determined by one-way ANOVA with post-hoc Tukey's correction for multiple comparisons; *P≤0.05, **P≤0.01, ***P≤0.001, and ****P<0.0001 compared to PBS control.

FIG. 5H is a volcano plot representing significance (−log 10) and fold change (log 2) for gene expression analysis in nAlb1-diABZI vs. PBS (n=4).

FIG. 5I is a volcano plot representing significance (−log 10) and fold change (log 2) for gene expression analysis in AP-diABZI vs. PBS (n=4).

FIG. 5J is a heat map of NanoString gene cluster matrices showing Z score fold changes for functional gene annotations. Results are the mean±SEM.

FIG. 5K is a heat map of NanoString gene cluster matrices showing Z score fold changes for biological signatures. Results are the mean t SEM.

FIG. 5L is a heat map of NanoString gene cluster matrices showing Z score fold changes for cell types. Results are the mean±SEM.

FIGS. 6A-G show that AP-diABZI activates a tumoricidal NK and T cell response. Flow cytometric analysis of orthotopic EMT6 breast tumors 24 h following two intravenous doses of AP-diABZI (1.25 μg, n=8), or PBS (n=7).

FIG. 6A shows tSNE plots of live cells in EMT6 tumors colored by cell population with relative expression level of Ki67, CD69, PD-1, and PD-L1 as indicated on heat map. DC: dendritic cell; Mφ: macrophage; NK: natural killer cell; MDSC: myeloid-derived suppressor cell.

FIG. 6B shows a heat map summarizing the fold change in the percentage of indicated cell population in EMT6 tumors.

FIG. 6C shows a heat map summarizing the fold change in the frequency of NK cells, CD8⁺ T cells, and CD4⁺ T cells expressing the indicated marker in EMT6 tumors.

FIG. 6D shows bar plots indicating an increase in CD8⁺ cells and the ratio of CD8⁺ to CD4⁺ FoxP3⁺ cells (as precent of CD3⁺ tumor cells).

FIG. 6E shows bar graphs quantifying Ki67⁺CD69⁺ and Ki67⁺PD-1⁺CD8⁺ T cells in EMT6 tumors.

FIG. 6F is a spleen phenotyping heat map of frequency of NK cells, CD8⁺ T cells, and CD4⁺ T cells.

FIG. 6G shows bar plots for activation and proliferation markers Ki67⁺CD69⁺ and Ki67⁺PD-1⁺ in CD8⁺ T cells and CD4⁺ T cells from spleens. *P≤50.05, **P≤0.01, ***P≤0.001, and ****P<0.0001 indicate a statistically significant difference between PBS and AP-diABZI treated groups as determined by Student's t-test. All results are the mean±SEM.

FIGS. 7A-7N show that albumin-hitchhiking STING agonists stimulate antitumor immunity in B16.F10 melanoma tumor model.

FIG. 7A schematically illustrates B16.F10 tumor inoculation and treatment schedule.

FIG. 7B show tumor growth curves. SEM with P value determined by two-way ANOVA with post-hoc Tukey's correction for multiple comparisons; ****P<0.0001 on day 18 for all groups compared to PBS.

FIG. 7C show spider plots of individual tumor growth curves.

FIG. 7D Kaplan-Meier survival plots (n=10-15). Anti-PD-L1 IgG (ICB) was injected I.P. at 100 μg and all nanobodies were injected I.V. at 1.25 μg of diABZI per injection. Kaplan-Meier survival curves of mice treated with indicated formulation using 1500 mm³ tumor volume as endpoint criteria with P value was determined by log-rank test; ****P<0.0001 compared to PBS control.

FIG. 7E is a heat map showing serum cytokine concentration in B16.F10 tumor bearing C57BL/6 female mice 4 hours after the first treatment (n=6-10).

FIG. 7F schematically illustrates B16.F10-OVA tumor inoculation, treatment schedule, and study end point for flow cytometry analysis (n=12). AP-diABZI was injected I.V. at 1.25 μg of diABZI per injection.

FIG. 7G is a bar graph showing tumor weight on day 15 for mice with B16.F10-OVA tumors treated with AP-diABZI or PBS.

FIG. 7H shows bar graphs quantifying the frequency of CD4⁺ and CD8⁺ T cells in the spleen at study endpoint.

FIG. 7I shows flow cytometric analysis of the frequency of CD69⁺ activated T cells,

FIG. 7J shows flow cytometric analysis of the frequency of CD44⁺CD62L⁻ effector memory T cells.

FIG. 7K shows flow cytometric analysis of the frequency of CD44⁻CD62L+ naive T cells.

FIG. 7L shows flow cytometric analysis of the frequency of CD44⁺CD62L⁺ central memory T cells.

FIG. 7M shows SIINFEKL/H-2 kB tetramer staining was performed to determine the frequency of OVA-specific CD8⁺ T cells in the spleen at study endpoint.

FIG. 7N shows representative flow cytometry dot plots demonstrating the distribution of CD8⁺ TEM (CD44⁺CD62L⁻) and TCM (CD44⁺CD62L⁺) within the OVA-specific (tetramer⁺) and non-OVA-specific (tetramer⁻) populations. *P≤0.05, **P≤0.01, ***P≤0.001, and ****P<0.0001 indicate a statistically significant difference between PBS and AP-diABZI treated groups as determined by Student's t-test. All results are the mean f SEM.

FIGS. 8A-8J show albumin-hitchhiking STING agonists improve immunotherapy responses in a model of lung metastatic melanoma and adoptive T cell transfer therapy.

FIG. 8A schematically illustrates B16.F10-LUC I.V. tumor inoculation, treatment schedule, and study end point for analysis of lung tumor burden (n=11-15). Anti-PD-L1 IgG (ICB) was injected 1.P. at 100 μg and all nanobodies were injected I.V. at 1.25 μg of diABZI per injection.

FIG. 8B shows representative images of lungs of mice treated as indicated.

FIG. 8C is a bar graph showing lung weights of mice treated as indicated.

FIG. 8D shows representative IVIS luminescent images of mice treated as indicated.

FIG. 8E is a bar graph quantifying average radiance from luciferase expressing B16.F10 within isolated lung tissue. P values determined by one-way ANOVA with post-hoc Tukey's correction for multiple comparisons; *P≤0.05, **P≤0.01, ***P≤0.001, and ****P<0.0001 compared to the PBS control.

FIG. 8F shows representative images of H&E stained lung sections from mice with B16.F10 lung metastases treated as indicated or lungs from healthy mice (control).

FIGS. 8G-J show the evaluation of AP-diABZI as an adjuvant therapy for adoptive OT-I T cell transfer therapy in a B16.F10-OVA model (n=15).

FIG. 8G schematically illustrates B16.F10-OVA tumor inoculation and of treatment schedule with OT-I transfer (0.5 million OT-I T cells) either on day 9 (OT-I alone or one dose (1.25 μg) AP-diABZI pre-treatment) or day 15 (three dose AP-diABZI pre-treatment).

FIG. 8H shows tumor growth curves. P value determined by two-way ANOVA with post-hoc Tukey's correction for multiple comparisons; ****P<0.0001 on day 17 for all groups compared to PBS.

FIG. 8I shows spider plots of individual tumor growth curves.

FIG. 8J shows Kaplan-Meier survival curves of mice treated with indicated formulation using 1500 mm³ tumor volume as endpoint criteria with P value was determined by log-rank test; ****P<0.0001 compared to PBS control. (CR=complete responder). All results are the mean±SEM.

FIG. 9A schematically illustrates treating healthy C57BU/6 female mice with PBS, 1.25 μg nAlb-diABZI, or 1.25 μg AP-diABZI.

FIG. 9B graphically shows the body weight change of mice during treatments.

FIG. 9C shows bar graphs quantifying plasma cytokines 4 hours after the first treatment and 24 hours after the final treatment (n=3-5, *P≤50.05, ***P≤0.001, ****P<0.0001 by one-way ANOVA compared to PBS).

FIG. 9D shows bar graphs assessing blood biochemistry of healthy C57BL/6 mice. After 3 treatments, mice were euthanized and blood samples were collected to determine changes in RBCs, white blood cells (WBCs), neutrophils, platelets, and lymphocytes. Serum samples were also used to analyze liver and kidney function, by measuring changes in ALT/AST, blood urea nitrogen (BUN), and creatinine. Tissue was sectioned, H&E stained, and imaged. Images use a scale bar of 200 μm. No significant changes were observed. (*P≤0.05, **P:s0.01, and ***P≤0.001 compared to PBS by one-way ANOVA).

FIG. 9E shows stained tissues for immunohistochemistry analysis of healthy C57BL/6 mice. After 3 treatments, mice were euthanized and blood samples were collected to determine changes in RBCs, white blood cells (WBCs), neutrophils, platelets, and lymphocytes. Serum samples were also used to analyze liver and kidney function, by measuring changes in ALT/AST, blood urea nitrogen (BUN), and creatinine. Tissue was sectioned, H&E stained, and imaged. Images use a scale bar of 200 μm. No significant changes were observed. (*P≤0.05, **P≤0.01, and ***P≤50.001 compared to PBS by one-way ANOVA).

FIG. 10 shows a NanoString panel for evaluating STING activation markers within EMT6 tumor bearing Balb/C mice 24 h after three doses of nAlb-diABZI (1.25 μg) or PBS (n=3).

FIG. 11 shows tSNE plots of live cells in EMT6 tumors after three doses of nAlb-diABZI (1.25 μg) or PBS, colored by cell population with relative expression levels. DC: dendritic cell; Mφ: macrophage; NK: natural killer cell; MDSC: myeloid-derived suppressor cells.

FIG. 12A shows annotated matrices for functional gene annotations comparing PBS, nAlb-diABZI (1.25 μg), and AP-diABZI (1.25 μg) 24 hours after three doses (n=3-4). P values determined by one-way ANOVA with post-hoc Tukey's correction for multiple comparisons; *P≤50.05, **P≤0.01, ***P≤0.001, and ****P<0.0001 compared to PBS control.

FIG. 12B shows annotated matrices for biological signatures comparing PBS, nAlb-diABZI (1.25 μg), and AP-diABZI (1.25 μg) 24 hours after three doses (n=3-4). P³ values determined by one-way ANOVA with post-hoc Tukey's correction for multiple comparisons; *P≤0.05, **P≤0.01, ***P≤0.001, and ****P<0.0001 compared to PBS control.

FIG. 12C shows annotated matrices for cell types from 10360 Pan Cancer NanoString gene expression panel comparing PBS, nAlb-diABZI (1.25 μg), and AP-diABZI (1.25 μg) 24 hours after three doses (n=3-4). P values determined by one-way ANOVA with post-hoc Tukey's correction for multiple comparisons; *P≤0.05, **P≤0.01, ***IP≤0.001, and ****P<0.0001 compared to PBS control.

FIGS. 13A-13F show flow cytometric analysis of EMT6 tumor bearing female Balb/c mice 48 h following a single dose of nAlb-diABZI (1.25 μg, n=8), AP-diABZI (1.25 μg, n=8), or PBS (n=7).

FIG. 13A shows tSNE plots of live cells in EMT6 tumors, colored by cell population with relative expression level of Ki67, CD69, PD-1, and PD-L1 as indicated on heat map. DC: dendritic cell: Mφ: macrophage; NK: natural killer cell; MDSC: myeloid-derived suppressor cell.

FIG. 13B shows bar graphs analyzing the frequency of live CD45⁻ cells and frequency of PD-L1 and Ki67 expressing CD45⁻ cells in the tumor. P values determined by one-way ANOVA with post-hoc Tukey's correction for multiple comparisons to PBS; *P≤0.05, **P≤0.01, ***P≤0.001, and ****P<0.0001 compared to the PBS control.

FIG. 13C is a heat map summarizing the fold change in the percentage of indicated cell population and of NK cells, CD8⁺ T cells, and CD4⁺ T cells expressing the indicated marker in EMT6 tumors. P values determined by one-way ANOVA with post-hoc Tukey's correction for multiple comparisons to PBS; *P≤0.05, **P≤0.01, ***P≤0.001, and ****P<0.0001 compared to the PBS control.

FIG. 13D is a heat map summarizing the fold change in the frequency of NK cells, CD8⁺ T cells, and CD4⁻ T cells expressing the indicated marker in EMT6 tumors. P values determined by one-way ANOVA with post-hoc Tukey's correction for multiple comparisons to PBS; *P≤0.05, **P≤0.01, ***P≤0.001, and ****P<0.0001 compared to the PBS control.

FIGS. 13E-13F show representative flow cytometry dot plots summarizing the distribution of Ki67⁺to CD69⁺ and PD-1⁺ CD8⁺ T cells and CD4⁺ T cells in EMT6 tumors. All results are the mean SEM.

FIG. 14A shows a spleen heat map summarizing the fold change in the percentage of indicated cell population 48 h after treatment with one dose of nAlb-diABZI (1.25 μg, n=8), AP-diABZI (1.25 μg, n=8), or PBS (n=6) from EMT6 tumor bearing Balb/c mice. P values determined by one-way ANOVA with post-hoc Tukey's correction for multiple comparisons to PBS; *P≤0.05, **P≤0.01, ***P≤0.001, and ****P<0.0001 compared to the PBS control.

FIG. 14B shows a spleen heat map summarizing the frequency of NK cells, CD8⁺ T cells, and CD4⁺ T cells expressing the indicated markers 48 h after treatment with one dose of nAlb-diABZI (1.25 μg, n=8), AP-diABZI (1.25 μg, n=8), or PBS (n=6) from EMT6 tumor bearing Balb/c mice. P values determined by one-way ANOVA with post-hoc Tukey's correction for multiple comparisons to PBS; *P≤0.05, **P≤0.01, ***P≤0.001, and ****P<0.0001 compared to the PBS control.

FIG. 15 shows tSNE plots of live cells from female EMT6 tumor bearing Balb/c mice after two doses of AP-diABZI (1.25 μg) or PBS. Maps are colored by indicated cell populations with relative expression levels. DC: dendritic cell, Mφ: macrophage; NK: natural killer cell; MDSC: myeloid-derived suppressor cells.

FIG. 16A is a heat map summarizing the fold change in the frequency of NK cells, CD8⁺ T cells, and CD4⁺ T cells expressing the indicated markers 24 h after treatment with either one or three doses of nAlb-diABZI (1.25 μg, n=6-8) or PBS (n=6-7) from EMT6 tumor bearing Balb/c mice in tumors. P values determined by one-way ANOVA with post hoc Tukeys correction for multiple comparisons to PBS; *P≤0.05, **P≤0.01, ***P≤0.001, and ****P<0.0001 compared to the PBS control matched from the day of the same dose.

FIG. 16B is a heat map summarizing the fold change in the frequency of NK cells, CD8⁺ T cells, and CD4⁺ T cells expressing the indicated markers 24 h after treatment with either one or three doses of nAlb-diABZI (1.25 μg, n=6-8) or PBS (n=6-7) from EMT6 tumor bearing Balb/c mice in spleens. P values determined by one-way ANOVA with post hoc Tukeys correction for multiple comparisons to PBS; *P≤0.05, **P≤50.01, ***P≤0.001, and ****P<0.0001 compared to the PBS control matched from the day of the same dose.

FIG. 17 shows serum cytokine/chemokine concentrations 4 hours post-injection with indicated nanobody-diABZI conjugate (n=6-10) in B16.F10 bearing female C57BL/6 mice. P values determined by one-way ANOVA with post-hoc Tukeys correction for multiple comparisons; *P≤0.05, **P≤0.01, ***P≤0.001, and ****P<0.0001 compared to PBS control.

FIG. 18A shows isothermal calorimetry (ITC) for nAlb at pH 7.5 and 5.5 in human serum albumin.

FIG. 18B shows isothermal calorimetry (ITC) for nAlb at pH 7.5 and 5.5 in recombinant mouse serum albumin.

FIG. 19 schematically illustrates the synthesis of DBCO-PEG11-diABZI (7).

FIG. 20A shows the ¹H NMR (500 MHz) spectrum of DBCO-PEGI 1-diABZI (7) in DMSO.

FIG. 20B shows the ¹³C NMR 151 MHz of DBCO-PEG11-diABZI (7) in DMSO.

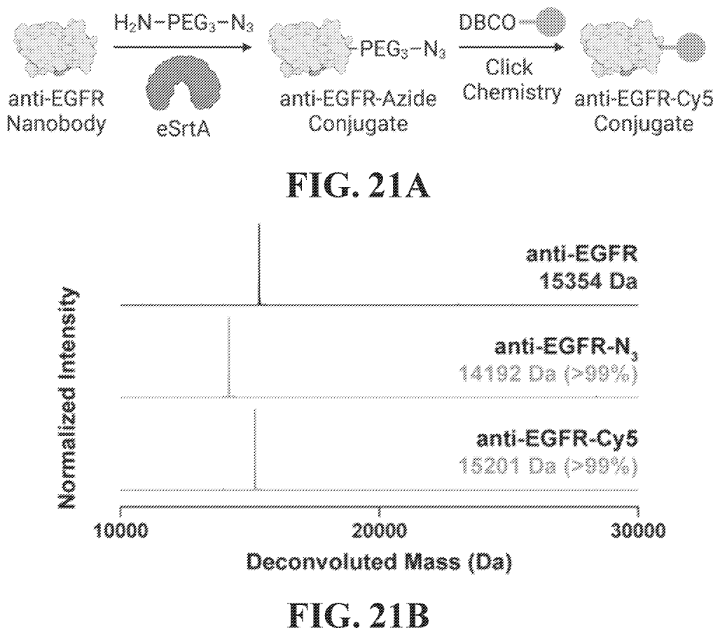

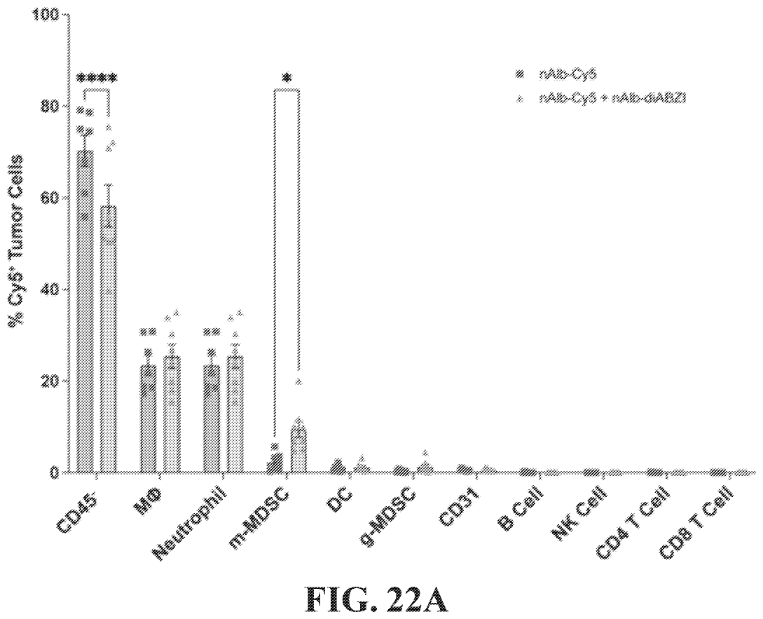

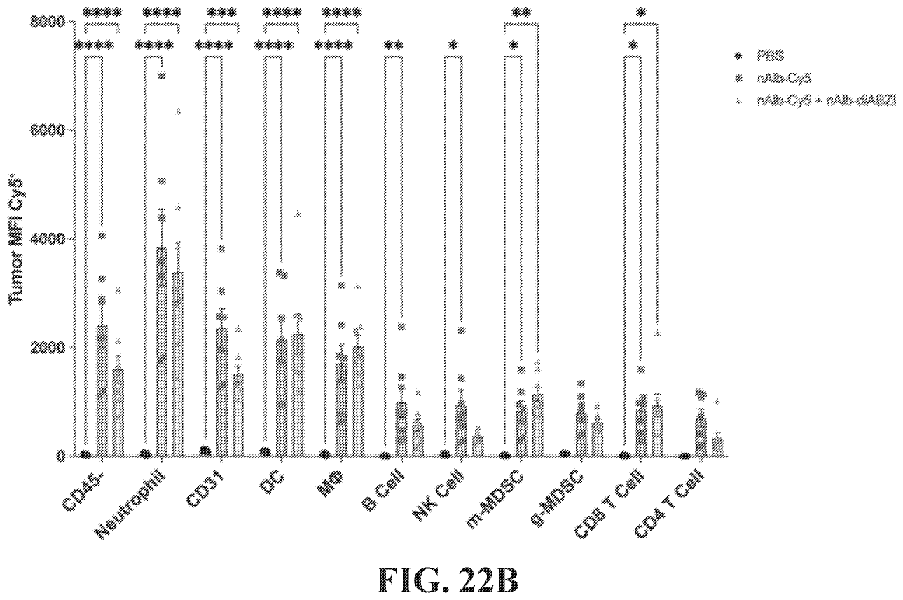

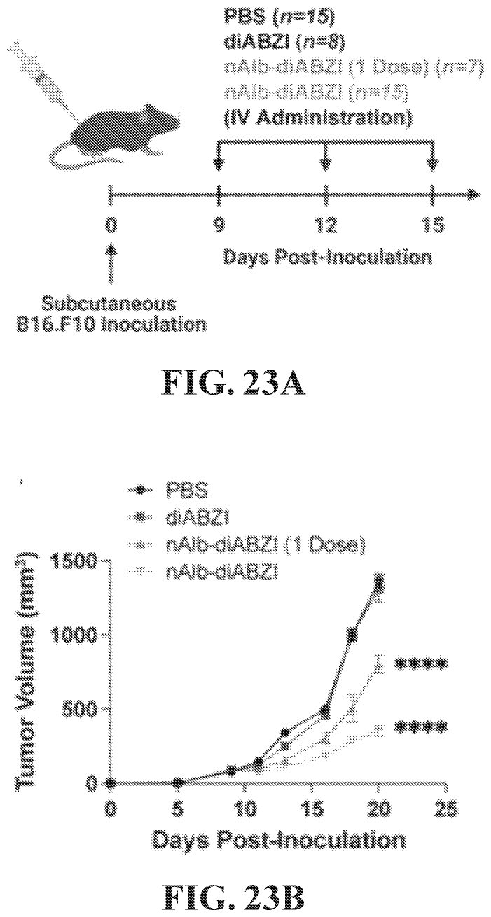

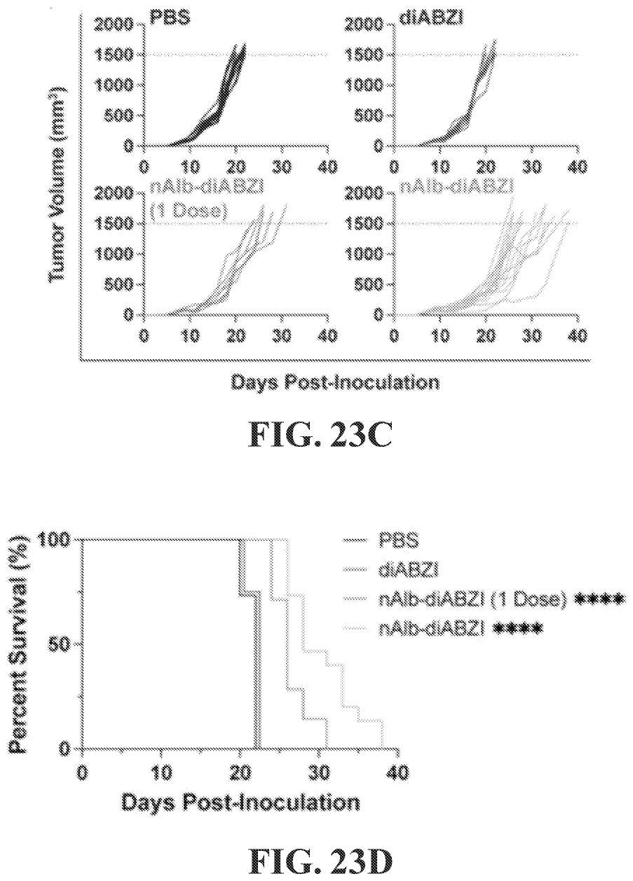

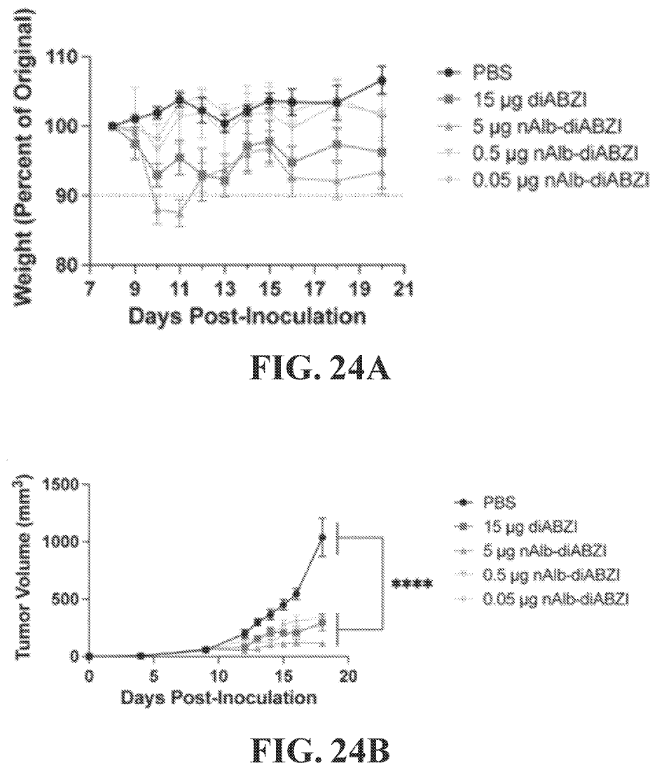

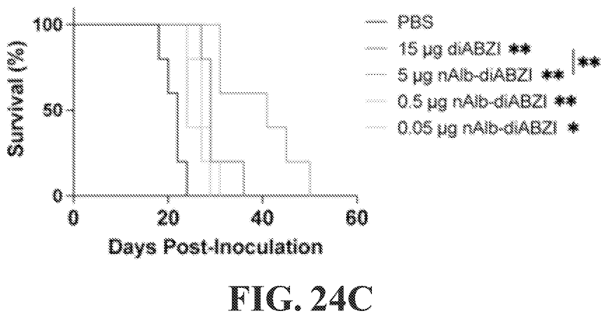

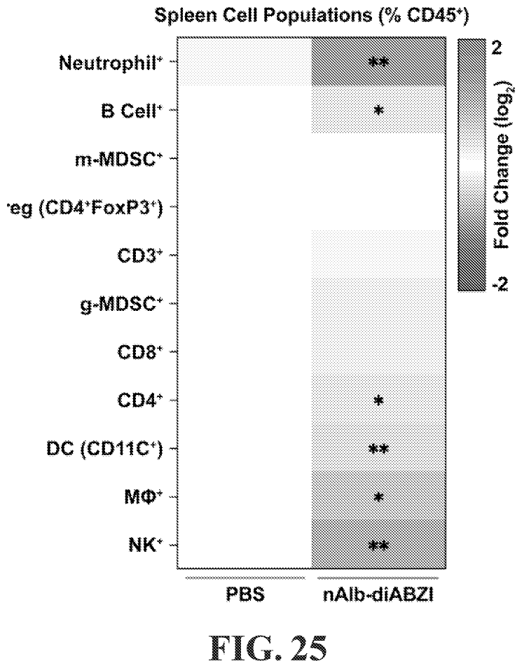

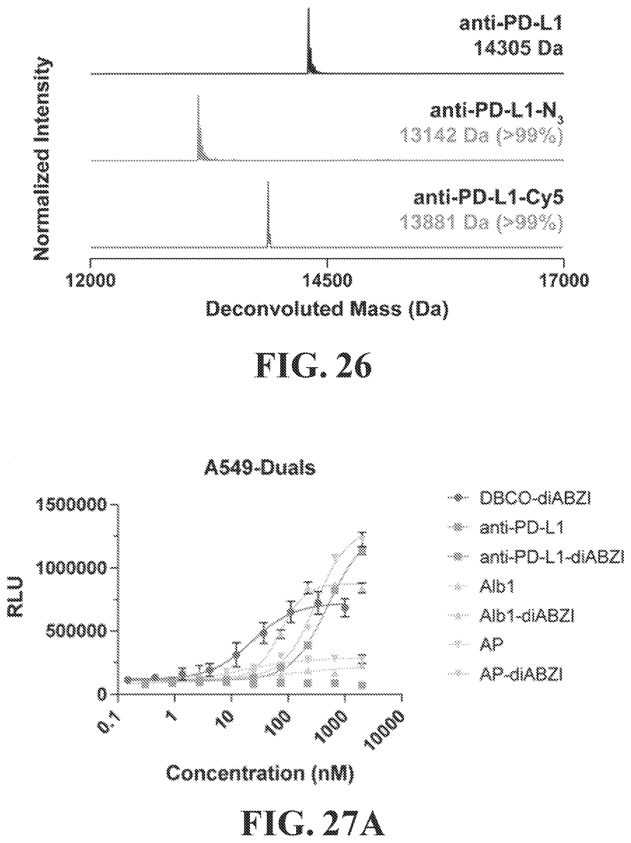

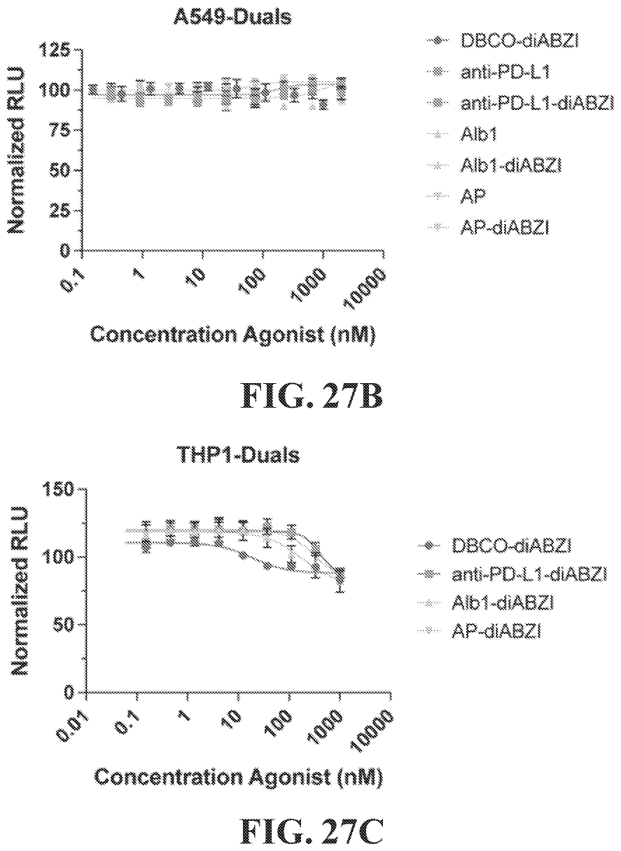

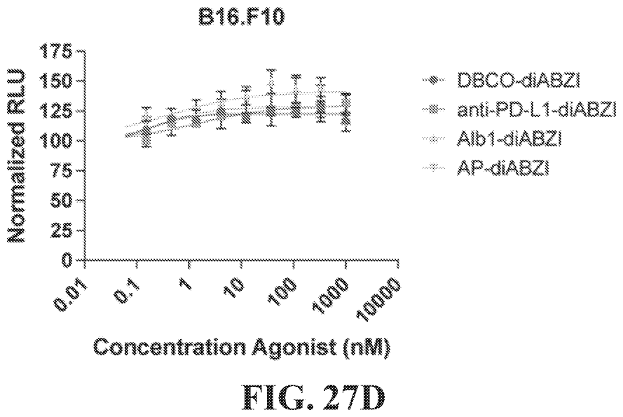

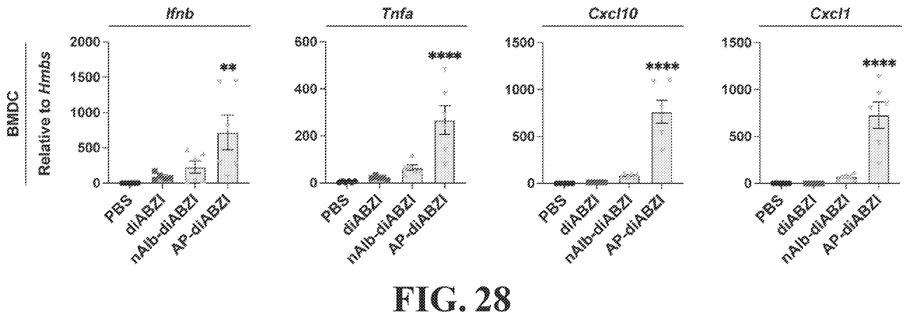

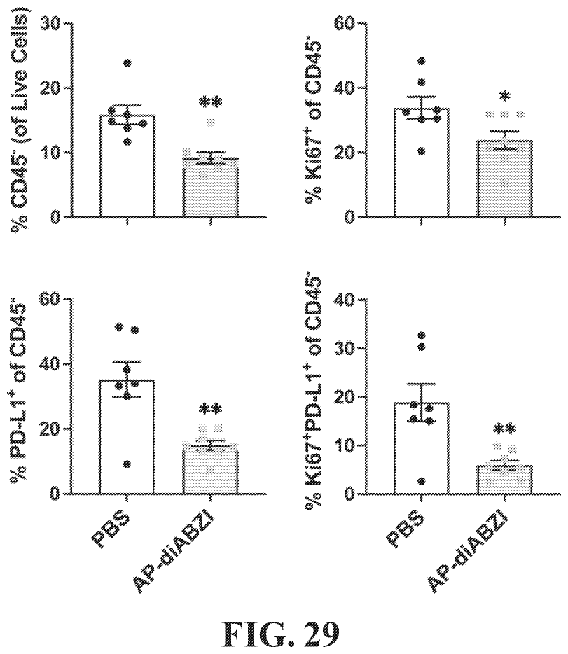

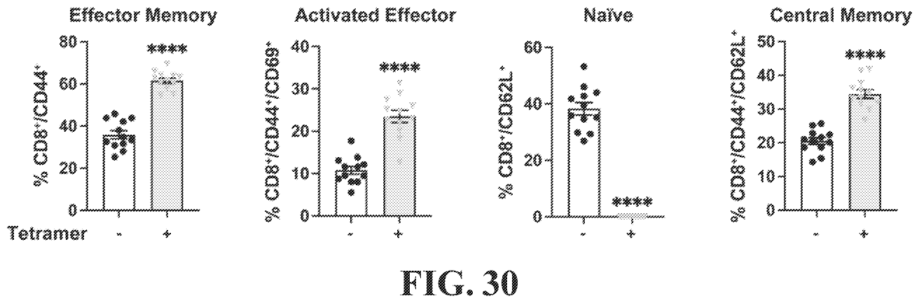

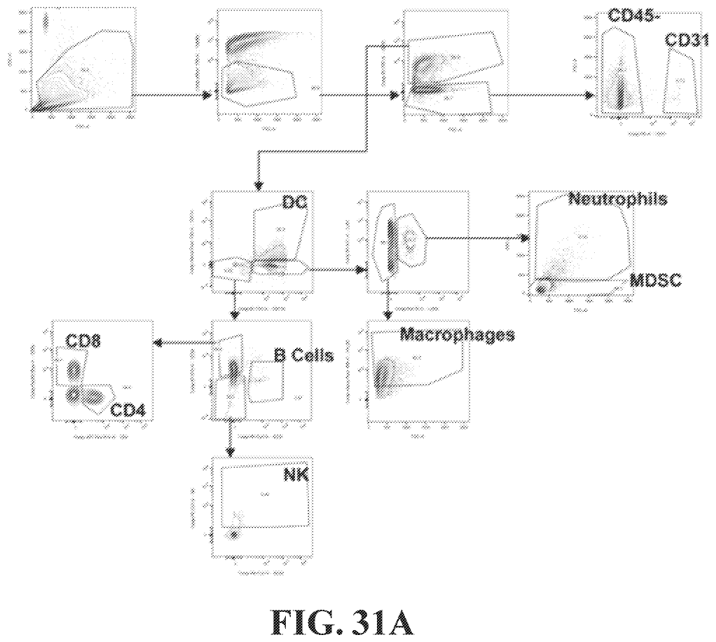

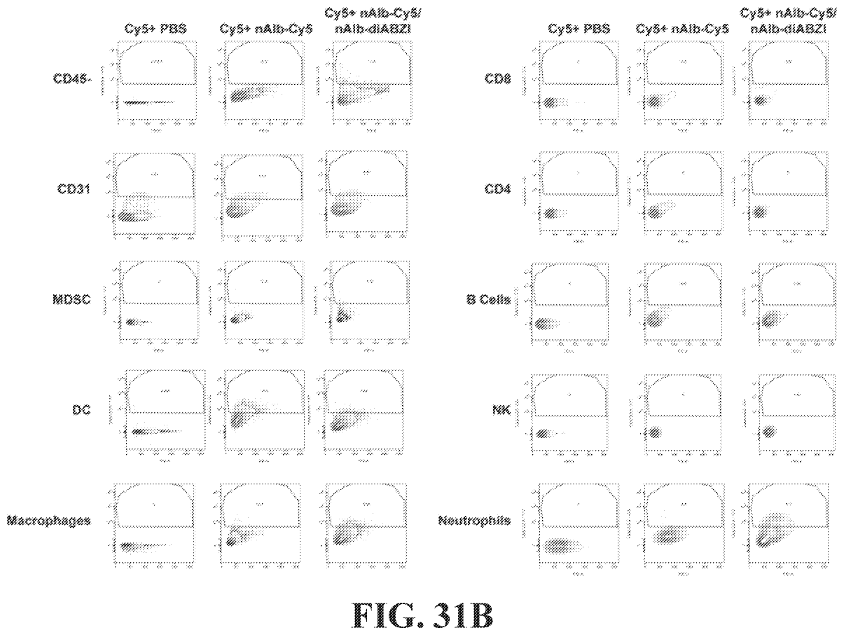

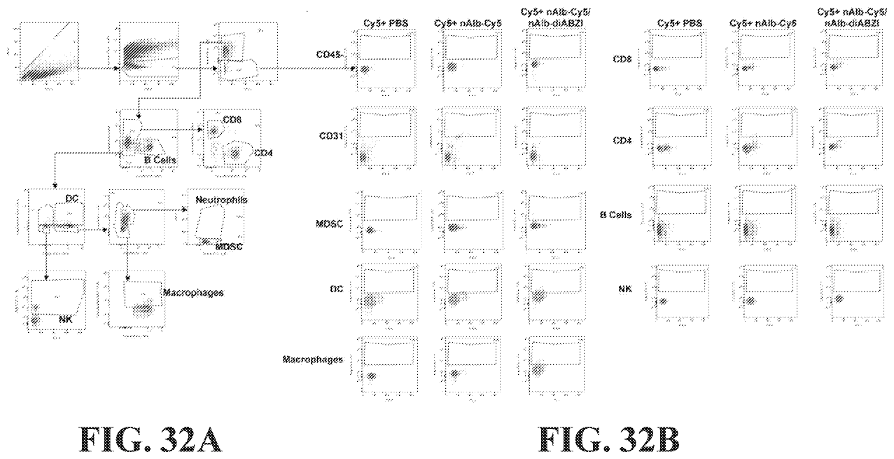

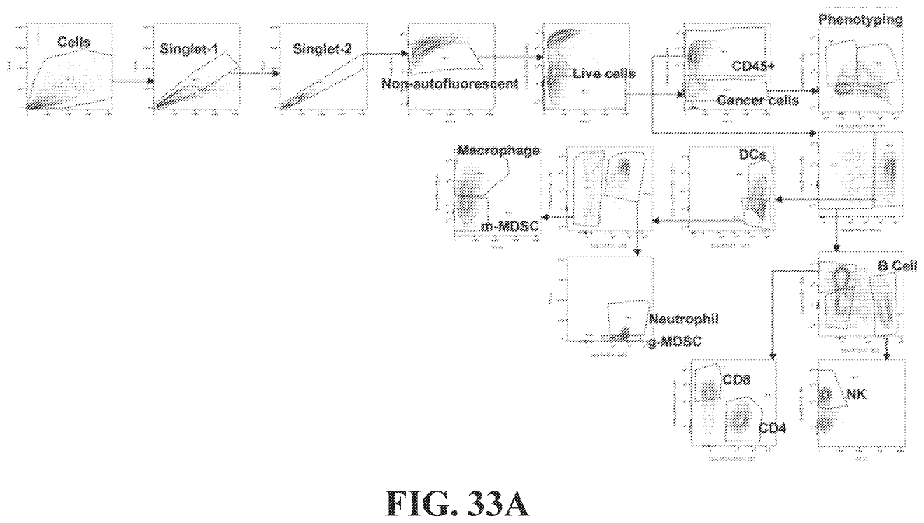

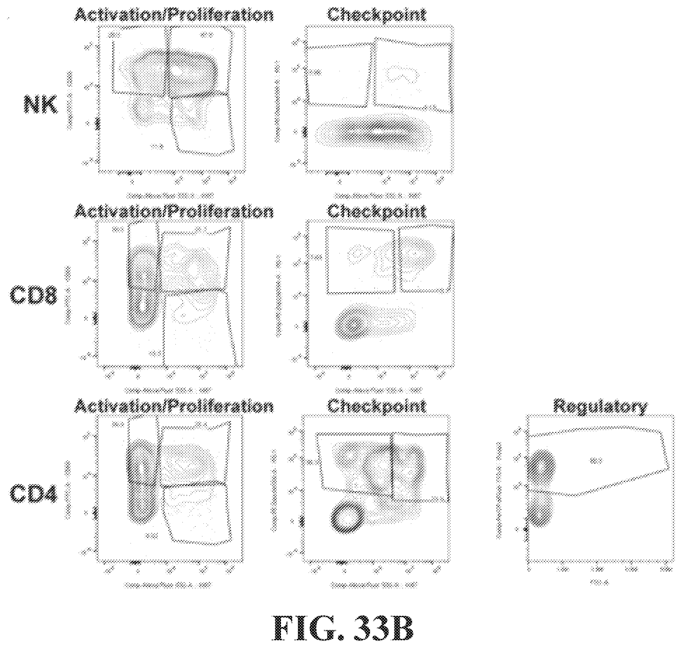

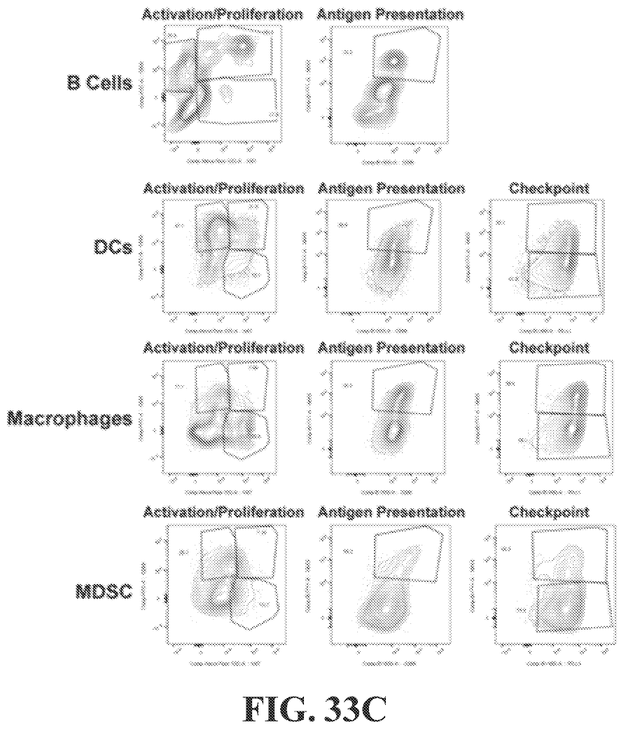

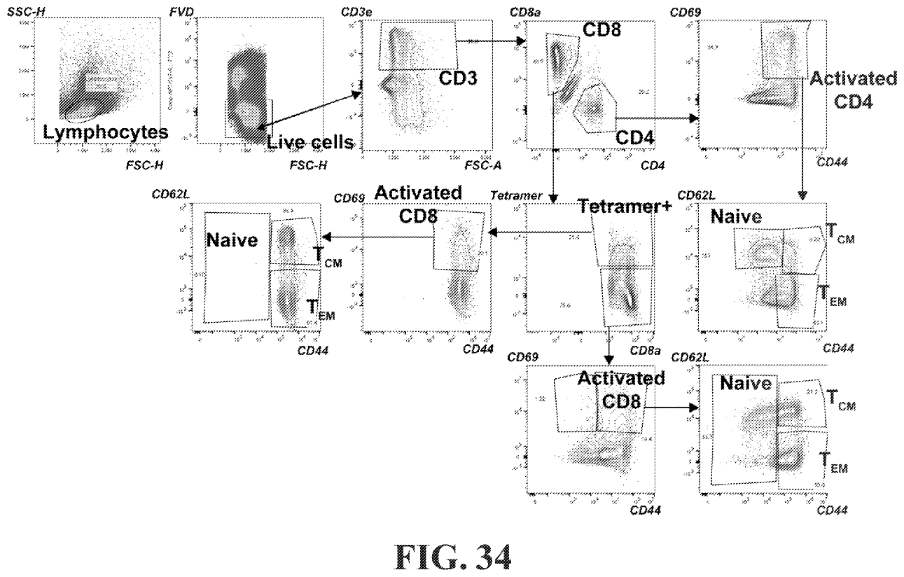

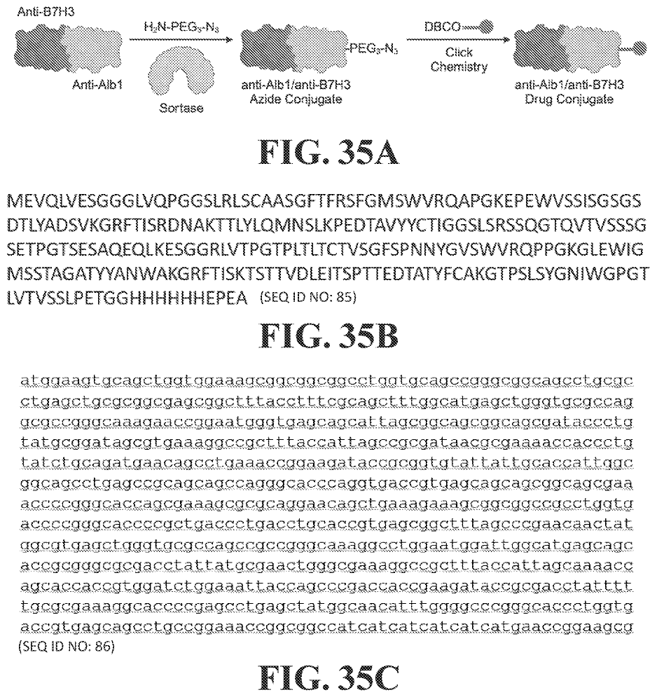

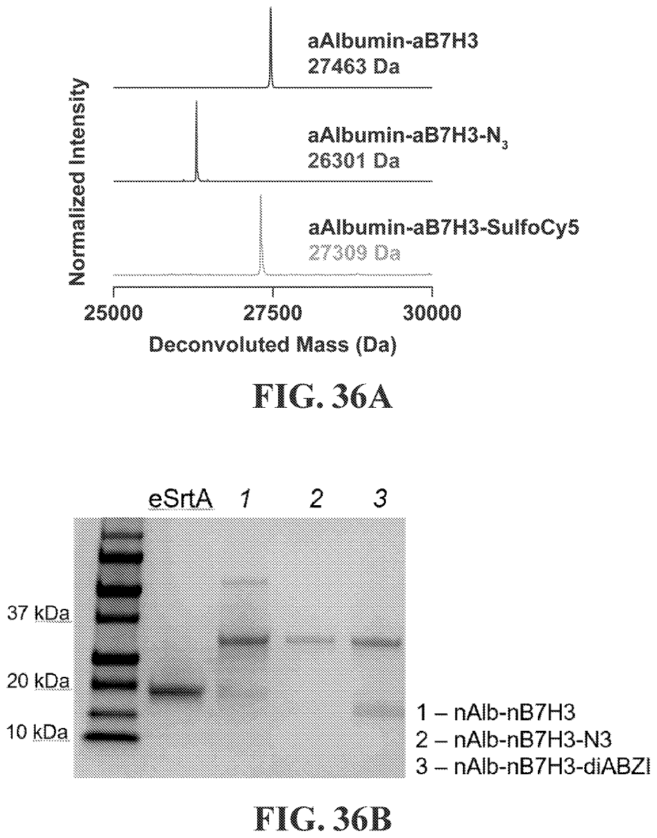

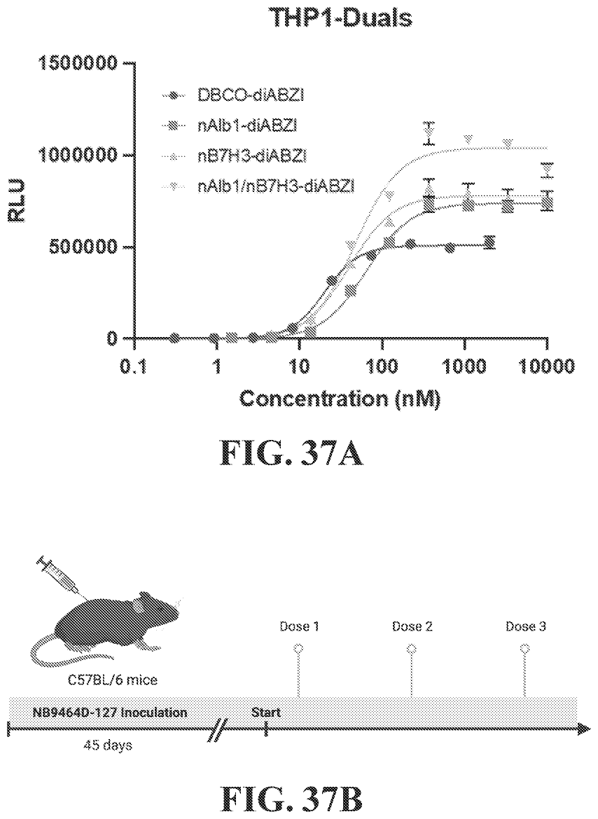

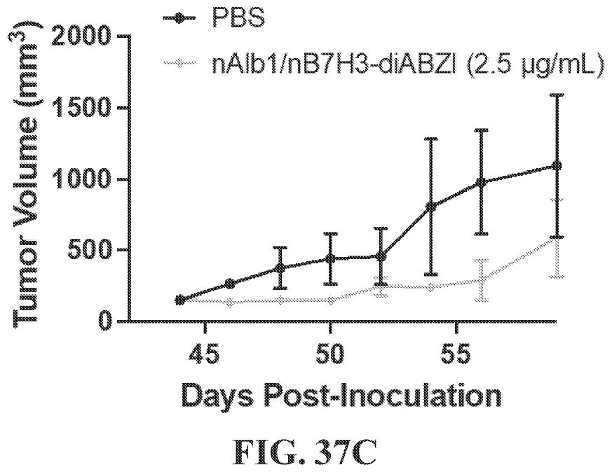

FIG. 21A schematically illustrates the synthesis of anti-EGFR⁻ (nEGFR) nanobody conjugates.

FIG. 21B shows ESI-MS characterization of anti-EGFR (nEGFR) nanobody conjugates.

FIG. 22A shows flow cytometric analysis for nAlb-Cy5 uptake in EMT6 tumors for populations percentages of total Cy5 positive tumor cells. DC: dendritic cell; Mφ: macrophage; MDSC: myeloid derived suppressor cell; NK: natural killer cell. P values determined by one-way ANOVA with post-hoc Tukey's correction for multiple comparisons; *P≤0.05, **P≤0.01, ***P≤50.001, and ****P<0.0001 compared to PBS control. Data shown as mean f SEM.

FIG. 22B shows flow cytometric analysis for nAlb-Cy5 uptake in EMT6 tumors for median fluorescent intensities (MFI) for each cell population. DC: dendritic cell; Mφ: macrophage; MDSC: myeloid derived suppressor cell; NK: natural killer cell. P values determined by one-way ANOVA with post-hoc Tukey's correction for multiple comparisons; *P≤0.05, **P≤0.01, ***P≤0.001, and ****P<0.0001 compared to PBS control. Data shown as mean f SEM.

FIG. 23A schematically illustrates B16.F10 tumor inoculation and treatment schedule. Free diABZI (1.25 μg) and nAlb-diABZI (1.25 μg) were injected I.V.

FIG. 23B shows tumor growth curves for mice with B16.F10 tumors treated as indicated. ****P<0.0001 on day 20 for all groups compared to PBS.

FIG. 23C shows spider plots of individual tumor growth curves for mice with B16.F10 tumors treated as indicated.

FIG. 23D shows Kaplan-Meier survival plots for mice with B16.F10 tumors treated as indicated. Data represented with error in SEM with P value determined by two-way ANOVA with post-hoc Tukey's correction for multiple comparisons. Kaplan-Meier survival curves of mice treated with indicated formulation using 1500 mm³ tumor volume as endpoint criteria with P value was determined by log-rank test; ****P<0.0001 compared to PBS control.

FIG. 24A graphically shows mouse weights after treatment as indicated. Free diABZI (15 μg) and nAlb-diABZI (5, 0.5, 0.05 μg) were injected I.V.

FIG. 24B shows tumor growth curves after treatment as indicated. Free diABZI (15 μg) and nAlb-diABZI (5, 0.5, 0.05 μg) were injected I.V. ****P<0.0001 on day 18 for all groups compared to PBS.

FIG. 24C shows Kaplan-Meier survival plots for mice with B16.F10 tumors treated as indicated. Data represented with error in SEM with P value determined by two-way ANOVA with post-hoc Tukey's correction for multiple comparisons. Kaplan-Meier survival curves of mice treated with indicated formulation using 1500 mm³ tumor volume as endpoint criteria with P value was determined by log-rank test; *P<0.05 and **P≤0.01 compared to PBS control, and **P≤0.01 noted between 15 μg diABZI and 5 μg nAlb-diABZI.

FIG. 25 is a heat map indicating fold changes differences of indicated cell population spleens from Balb/c mice bearing EMT6 tumors treated as indicated. *P≤0.05, **P≤0.01, ***P≤0.001, and ****P<0.0001 indicate a statistically significant difference between PBS and nAlb-diABZI treated groups as determined by Student's t-test, n=6 per group. All results are the mean f SEM.

FIG. 26 shows ESI-MS characterization for anti-PD-L1 nanobody conjugates.

FIG. 27A-D show in vitro activity of nanobody-diABZI conjugates in A549-Dual and THP1-Dual IFN-I reporter cells. All nanobody-diABZI conjugates were potently active in both reporter cell lines without evidence of cytotoxicity.

FIG. 27A shows in vitro dose-response curves by Cell-622 Titer Glo in A549-Dual reporter cells.

FIG. 27B graphically shows coupled toxicity by Cell-622 Titer Glo in A549-Dual reporter cells.

FIG. 27C graphically shows toxicity by Cell-622 Titer Glo in THP1-Dual reporter cells.

FIG. 27D graphically shows toxicity by Cell-622 Titer Glo in murine B16.F10 melanoma cells.

FIG. 28 graphically shows in vitro qPCR analysis of genes associated with STING activation in bone marrow derived dendritic cells (BMDCs) as analyzed 4 h post-treatment STING agonist as free diABZI or nanobody-diABZI conjugates at 0.25 μM (n=5-6). P values determined by one-way ANOVA with post-hoc Tukey's correction for multiple comparisons; **P≤0.01 and ****P<0.0001 compared to PBS control.

FIG. 29 graphically shows analysis of the frequency of live CD45⁻ cells, and the frequency of PD-L1 and Ki67expressing CD45⁻ cells within the tumor after two doses of AP-diABZI (1.25 μg, n=8) or PBS (n=7).*P≤0.05 and **P≤0.01 indicates a statistically significant difference between PBS and AP-diABZI treated groups as determined by Student's t-test. All results are the mean±SEM.

FIG. 30 graphically shows quantification of flow cytometric analysis presented in FIG. 7N showing the distribution of memory of OVA-specific (SIINFEKL/H-2 kB tetramer⁺) or non-SIINFEKL-specific (tetramer) CD8⁻ T cells. Represented bar plots include effector memory TEM (CD44⁺CD62L⁻), activated CD69⁺ effector memory TEM (CD44⁺CD69⁺CD62L⁻), naive (CD44⁻CD62L⁺), and central memory TCM (CD44⁺CD62L⁺). ****P<0.0001 indicates a statistically significant difference between tetramer negative and tetramer positive treated groups as determined by Student's t-test. All results are the mean±SEM.

FIG. 31A-B show representative flow cytometry dot plots showing gating strategy related to FIG. 2J.

FIG. 31A shows analysis of tumor cell populations present.

FIG. 31B shows analysis of relative uptake of Cy5 in each cell population.

FIGS. 32A-32B show representative flow cytometry dot plots and gating strategy for FIG. 2K.

FIG. 32A shows analysis of splenic cell populations present.

FIG. 32B shows analysis of relative uptake of Cy5 in each cell population.

FIG. 33A-33B show representative flow cytometry analysis for cell population mapping and immunophenotype determination within tumors and spleens from EMT6 bearing mice.

FIG. 33A shows cell population gating strategy.

FIG. 33B shows NK, CD8 T cell, and CD4 T cell activation, proliferation, and checkpoint markers.

FIG. 33C shows activation, proliferation, antigen presentation, and checkpoint markers for B cells, dendritic cells (DCs), macrophages, and MDSCs.

FIG. 34 shows representative flow cytometric gating strategy for spleen cells from B16.F10-OVA bearing C57BL/6 bearing female mice. Analysis determination of CD4, CD8 T cells, and SIINFEKL/H-2 kB stained tetramer CD8 T cells, as well as both activation and memory T cell markers.

FIG. 35A schematically illustrates the synthesis scheme of anti-albumin/anti-B7H3 fusion drug conjugate, the drug depicted can be any small molecule functionalized with a DBCO group.

FIG. 35B shows the amino acid sequence encoding anti-Albuminvanti-B7H3.

FIG. 35C shows the predicted DNA sequence encoding anti-Albumin/anti-B7H3.

FIG. 36A shows an ESI-MS spectrum depicting successful sythnesis of anti-albumin/anti-B7H3 conjugated to Cy5.

FIG. 36B is an image of an SDS-PAGE showing anti-albumin/anti-B7H3 conjugated to a small molecule drug, diABZI.

FIG. 37A graphically shows interferon-β (IFN-β) dose response to nanobody-drug conjugates in THP1-Dual reporter IFN-β cells.

FIG. 37B graphically shows treatment scheme depicting study conditions for NB9464D-127 (127 denotes GPC2 overexpressing line) in C57BL/6 mice. Doses were administered every 4 days.

FIG. 37C shows tumor volume curves until Day 59 when first tumor related death was observed in the PBS group.

### DETAILED DESCRIPTION

Described herein are albumin-hitchhiking, nanobody-STING agonist conjugates. “Albumin hitchhiking” can leverage molecular design of albumin binding chaperones to harness albumin's long circulation time and proclivity to accumulate at tumor sites via both passive and active transport processes. High-affinity anti-albumin nanobodies have been designed and prepared as site-selective enzymatic bioconjugation of STING agonists via biorthogonal chemistry. Employing a novel conjugatable diamidobenzimidazole (diABZI) STING agonist as a clinically relevant example, nanobody hitchhiking of STING agonists on serum albumin noticeably improves the pharmacological properties of these STING agonists and increases tumor tropism, leading to a reduction in tumor burden and improved therapeutic outcomes in multiple mouse tumor models.

Further demonstrated is the programmability of these nanobody-STING agonist conjugates for integrating tumor targeting and additional immunoregulatory functions through the development of a bispecific nanobody-drug conjugate that binds to both albumin and the immune checkpoint ligand PD-L1. An example bivalent nanobody carrier was used for STING agonist delivery, further increasing tumor accumulation while also inhibiting immunosuppressive PD-1/PD122 L1 interactions, resulting in a reprograming of the tumor microenvironment (TME) to a more immunogenic “hot” milieu and a priming of antitumor T cells that further potentiate responses to multiple immunotherapeutic modalities. Collectively, these albumin-hitchhiking, nanobody-STING agonist conjugates provide a multimodal and programmable platform for cancer immunotherapy with high translational potential.

### 1. Definitions

Unless otherwise defined, all technical and scientific terms used herein have the same meaning as commonly understood by one of ordinary skill in the art. In case of conflict, the present document, including definitions, will control. The materials, methods, and examples disclosed herein are illustrative only and not intended to be limiting. Methods and materials similar or equivalent to those described herein can be used in practice or testing of the disclosed invention. All publications, patent applications, patents and other references mentioned herein are incorporated by reference in their entirety.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are contemplated, and for the range 1.5-2, the numbers 1.5, 1.6, 1.7, 1.8, 1.9, and 2 are contemplated.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition. Cambridge University Press, Cambridge, 1987; the entire contents of each of which are incorporated herein by reference.

The term “alkyl,” as used herein, means a straight or branched, saturated hydrocarbon chain. The term “lower alkyl” or “C₁₋₆alkyl” means a straight or branched chain hydrocarbon containing from 1 to 6 carbon atoms. The term “C₁₋₄alkyl” means a straight or branched chain hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkenyl,” as used herein, means a straight or branched, hydrocarbon chain containing at least one carbon-carbon double bond.

The term “alkynyl,” as used herein, means a straight or branched, hydrocarbon chain containing at least one carbon-carbon triple bond.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

The term “drug” refers to a substance that can act on a cell, virus, tissue, organ, organism, or the like, to create a change in the functioning of the cell, virus, tissue, organ, or organism. Examples of drugs include, but are not limited to, chemotherapeutics, anti-inflammatory drugs, and immunomodulating drugs. A drug is capable of treating and/or ameliorating a condition or disease, or one or more symptoms thereof, in a subject. Drugs of the present disclosure also include prodrug forms of the agent.

The term “effective dosage” or “therapeutic dosage” or “therapeutically effective amount” or “effective amount,” as used herein, refers to an amount sufficient to effect beneficial or desirable biological and/or clinical results, to modulate a biological process, and/or treat a disease or one or more of its symptoms and/or to prevent or reduce the risk of the occurrence or reoccurrence of the disease or disorder or symptom(s) thereof. A therapeutically effective amount is also one in which any toxic or detrimental effects of substance are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount. In reference to treating a disease or disorder an effective or therapeutically effective amount can include an amount sufficient to, among other things, improve survival from a disease, such as cancer.

The term “halogen” or “halo,” as used herein, means Cl, Br, I, or F.

The term “haloalkyl,” as used herein, means an alkyl group, as defined herein, in which one, two, three, four, five, six, seven or eight hydrogen atoms are replaced by a halogen.

The term “nanobody,” as used herein, refers to an antibody fragment including a single monomeric variable antibody domain, such as a single variable domain of a heavy chain. Nanobodies typically have molecular weights of between 12 kDa and 15 kDa and can include peptide chains of from 90 to 120 amino acids. Nanobodies can be obtained from heavy-chain antibodies found in camelid species and from cartilaginous fish species (e.g., sharks). Nanobodies can also be obtained from variable domains of common IgG derived from humans or mice. It should be understood that while most nanobodies are derived from heavy-chain variable domains of antibodies, nanobodies can also be derived from light-chain antibody domains.

To create, e.g., a camelid nanobody immune library, camelids can be immunized against a molecule of interest (albumin, checkpoint immune ligand, etc.). mRNA of the camelids' peripheral blood mononuclear cells can then be converted into cDNA. PCR can then be employed to amplify the VHH genes. These immune VHH genes can then be cloned into a phage display vector. Phages can then be generated using E. coli strains such as TG1. Phage libraries can then be panned against immobilized antigens to select for nanobodies that selectively bind the antigen with high affinity. The panned libraries can be used for reinfection of E. coli to obtain specific clones. Further description of nanobodies can be found in S. Muyldermans, A guide to: generation and design of nanobodies, FEBS J. 2021 April; 288(7): 2084-2102, which is incorporated by reference herein in its entirety.

“Subject” and “patient” as used herein interchangeably refers to any vertebrate, including, but not limited to, a mammal that wants or is in need of the herein described conjugates or methods. The subject may be a human or a non-human. The subject may be a vertebrate. The subject may be a mammal. The mammal may be a primate or a non-primate. The mammal can be a non-primate such as, for example, cow, pig, camel, llama, hedgehog, anteater, platypus, elephant, alpaca, horse, goat, rabbit, sheep, hamsters, guinea pig, cat, dog, rat, and mouse. The mammal can be a primate such as a human. The mammal can be a non-human primate such as, for example, monkey, cynomolgous monkey, rhesus monkey, chimpanzee, gorilla, orangutan, and gibbon. The subject may be of any age or stage of development, such as, for example, an adult, an adolescent, or an infant. The subject may be male. The subject may be female. In some embodiments, the subject has a specific genetic marker. The subject may be undergoing other forms of treatment.

“Sortase” refers to an enzyme that recognizes a sortase recognition site in a protein and cleaves a peptide bond therein, forming a stable intermediate that joins the catalytic thiol of sortase to the carboxyl group of an amino acid within the recognition site via a thioester bond. An example sortase is Sortase A (SrtA).

As used herein, the term “specifically binds” is generally meant that a molecule (e.g., a nanobody or conjugate thereof) binds to a target molecule when it binds to that target molecule more readily than it would bind to a random, unrelated target. For example, nanobodies disclosed herein can specifically bind to a target molecule with nanomolar affinity. “Specific binding” does not necessarily require (although it can include) exclusive binding to a target molecule or epitope thereof.

The term “substituted” refers to a group that may be further substituted with one or more non-hydrogen substituent groups. Substituent groups include, but are not limited to, halogen, ═O (oxo), ═S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

Terms such as “alkyl,” “cycloalkyl,” “alkylene,” etc. may be preceded by a designation indicating the number of atoms present in the group in a particular instance (e.g., “C₁₋₄alkyl,” “C₃₋₆cycloalkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

### 2. Conjugates

Provided herein are conjugates that can advantageously bind to albumin. The conjugate includes a nanobody portion, a drug, and a linker attaching the nanobody portion to the drug. The nanobody portion can include at least one nanobody that can specifically bind albumin, which can allow the conjugate to bind albumin. The ability to bind albumin can provide advantageous benefits to the conjugate including, but not limited to, improved pharmacokinetics and pharmacodynamics of the attached drug.

The disclosed conjugates may exist as salts, such as pharmaceutically acceptable salts. The term “pharmaceutically acceptable salt” refers to salts or zwitterions of the conjugates which are water or oil-soluble or dispersible, suitable for administration to a subject (e.g., treatment of disorders) without undue toxicity, irritation, and allergic response, commensurate with a reasonable benefit/risk ratio and effective for their intended use. The salts may be prepared during the final isolation and purification of the conjugates or separately by reacting an amino group of the conjugates with a suitable acid. For example, the conjugate may be dissolved in a suitable solvent and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate. maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric and the like. Amino groups of the conjugates may also be quaternized with alkyl chlorides, bromides and iodides such as methyl, ethyl, propyl, isopropyl, butyl, lauryl, myristyl, stearyl and the like.

Basic addition salts may be prepared during the final isolation and purification of the disclosed conjugates by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

### A. Nanobody Portions

The nanobody portion includes at least one nanobody. In some embodiments, the nanobody portion includes a first nanobody and a second nanobody. The first nanobody and the second nanobody can specifically bind to different target molecules. A nanobody portion including a first nanobody and a second nanobody can be considered bivalent, and as a result can instill bivalency to the conjugate. Bivalency of the conjugate can aid in overall targeting of the conjugate and can aid in inhibition of signaling pathways associated with a target molecule.

The first nanobody can be an albumin-binding nanobody. The albumin-binding nanobody is capable of specifically binding albumin, and thus can instill in the conjugate the ability to specifically bind albumin. The albumin-binding nanobody can have a binding affinity (K_{d}) to albumin of less than or equal to 100 nM at a pH of about 7 to about 8, such as less than or equal to 75 nM, less than or equal to 50 nM, less than or equal to 25 nM, less than or equal to 10 nM, or less than or equal to 1 nM at a pH of about 7 to about 8. In some embodiments, the albumin-binding nanobody has a K_{d} to albumin of greater than or equal to 0.1 nM. greater than or equal to 0.2 nM, greater than or equal to 0.4 nM, greater than or equal to 0.5 nM, greater than or equal to 0.6 nM. greater than or equal to 0.7 nM, greater than or equal 0.8 nM, greater than or equal to 0.9 nM, or greater than or equal to 1 nM at a pH of about 7 to about 8. In some embodiments, the albumin-binding nanobody has a K_{d} to albumin of about 0.1 nM to about 100 nM, such as about 0.2 nM to about 90 nM, about 1 nM to about 100 nM, or about 0.1 nM to about 50 nM. In some embodiments, the albumin-binding nanobody does not covalently bind to albumin. Binding affinity of nanobodies can be measured via techniques known within the art, such as isothermal calorimetry (ITC). Further description of using ITC for measuring binding affinity can be found in the Examples below.

The albumin-binding nanobody can include any nanobody suitable for specifically binding albumin as described herein. In some embodiments, the albumin-binding nanobody includes an amino acid sequence selected from the group consisting of SEQ ID NO: 5 to SEQ ID NO: 79 and SEQ ID NO: 89. In some embodiments, the albumin-binding nanobody includes an amino acid sequence of SEQ ID NO: 89.

As detailed above, the nanobody portion can include a second nanobody. In some embodiments, the second nanobody is capable of specifically binding to an immune checkpoint ligand. In some embodiments, the immune checkpoint ligand is present on a cell surface. Example immune checkpoint ligands include, but are not limited to, 72 CTLA-4, PD-1, PD-L1, B7-H3, B7-H4, HVEM, GITRL, CD80/86, CD155, PD-L1, Galectin 9, LAG3, TIM3, VISTA, TIGIT, PD1, and GITR. In some embodiments, the immune checkpoint ligand is 72 CTLA-4, PD-I, PD-L1, B7-H3, or B7-H4. In some embodiments, the immune checkpoint ligand is PD-1, PD-L1, B7-H3, or B7-H4. In some embodiments, the immune checkpoint ligand is PD-L1 or B7-H3. The second nanobody can have a K_{d} to an immune checkpoint ligand as described above for the albumin-binding nanobody to albumin.

In some embodiments, the second nanobody includes an amino acid sequence selected from the group consisting of SEQ ID NO: 82, SEQ ID NO: 87, SEQ ID NO: 88, and SEQ ID NO: 90. In some embodiments, the second nanobody includes an amino acid sequence selected from the group consisting of SEQ ID NO: 88 and SEQ ID NO: 90.

The first nanobody can be attached to the second nanobody through a nanobody linker. The nanobody linker can be a peptide linker. In some embodiments, the nanobody linker includes an amino acid sequence of SEQ ID NO: 84.

In some embodiments, the nanobody portion includes a first nanobody that is capable of specifically binding to albumin and a second nanobody that is capable of specifically binding to an immune checkpoint ligand. In some embodiments, the nanobody portion includes a first nanobody including an amino acid sequence selected from the group consisting of SEQ ID NO: 5 to SEQ ID NO: 79 and SEQ ID NO: 89, and a second nanobody including an amino acid sequence selected from the group consisting of SEQ ID NO: 82, SEQ ID NO: 87, SEQ ID NO: 88, and SEQ ID NO: 90. In some embodiments, the nanobody portion includes an amino acid sequence selected from the group consisting of SEQ ID NO: 83 and SEQ ID NO: 85.

The nanobodies disclosed herein can be commercially purchased or provided by recombinant expression.

### B. Drugs

Any suitable drug can be used in the disclosed conjugates. Example drugs include, but are not limited to, an immunomodulator, an agonist, an antagonist, an inhibitor, or a hormone. In some embodiments, the drug comprises an immunomodulator or a hormone. In some embodiments, the drug comprises an immunomodulator. In some embodiments, the drug is an immunomodulator.

The immunomodulator can be a STING agonist. Example STING agonists include, but are not limited to, a dimeric amidobenzimidazole (diABZI), 2′3′ cGAMP, 2′2′ cGAMP, 3′2′ cGAMP, 3′3′ cGAMP, c-di-GMP, c-di-AMP, ADU-S100, cIAMP 2-5, and ML RR-S2 CDA. In some embodiments, the STING agonist is a non-nucleotide STING agonist. In some embodiments, the STING agonist is diABZI.

In some embodiments, diABZI is:

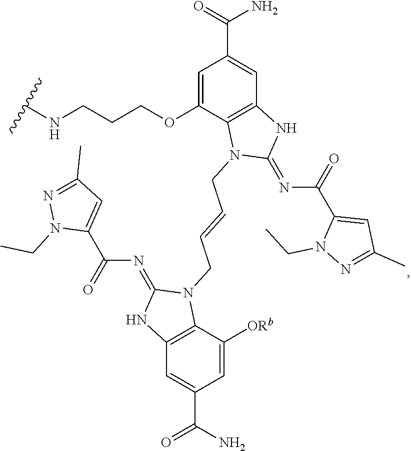

wherein:
R^{b} is methyl,

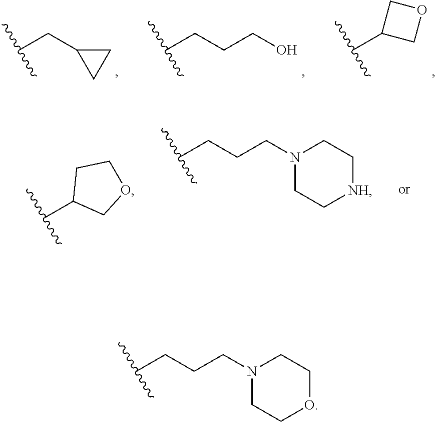

In some embodiments, R^{b} is methyl. Further description of diABZI can be found in PCT/US2023/076732, which is incorporated fully herein by reference.

### C. Linkers

The nanobody portion is attached (e.g., covalently) to the drug through the linker. The linker includes a nanobody linking moiety. The nanobody linking moiety can be attached to the nanobody portion. In some embodiments, the nanobody linking moiety is located at the C-terminus of a nanobody of the nanobody portion. The nanobody linking moiety can include a sortase recognition site. For example, the nanobody linking moiety can include an amino acid sequence of LPXT (SEQ ID NO:1), wherein X is any amino acid. In some embodiments, the nanobody linking moiety includes an amino acid sequence selected from the group consisting of SEQ ID NO: 1, SEQ ID NO: 2, SEQ ID NO: 3, and SEQ ID NO: 4. In some embodiments, the nanobody linking moiety and/or amino acid sequence thereof is described as part of a sequence encoding a nanobody. The nanobody linking moiety may also include a hydrophilic moiety, such as polyethylene glycol.

In embodiments where the nanobody portion includes a first nanobody and a second nanobody, the nanobody linking moiety can be attached to one of the nanobodies. For example, in embodiments that include a first nanobody (e.g., albumin-binding nanobody) and a second nanobody (e.g., immune checkpoint ligand binding nanobody), the nanobody linking moiety can be attached to either the first nanobody or the second nanobody. In some embodiments, the nanobody linking moiety is attached to the second nanobody (e.g., where the nanobody portion includes two nanobodies).

The linker can also include a hydrophilic moiety. The hydrophilic moiety can attach the nanobody linking moiety to the drug. Due to the hydrophobicity of the drug, such as diABZI, the linker can advantageously aid in solubilizing the drug. An example hydrophilic linker includes a hydrophilic polymer such as polyethylene glycol (PEG). Accordingly, in some embodiments, the linker includes a PEG moiety that, e.g., attaches the nanobody linking moiety to the drug. The PEG moiety can include a varying number of ethylene glycol repeats. For example, the PEG moiety can include 3 to 20 ethylene glycol repeats, such as 4 to 20, 5 to 20, 6 to 20, 7 to 20, 8 to 20, 9 to 20, or 10 to 20. Tn some embodiments, the PEG moiety includes greater than 2 ethylene glycol repeats, greater than 3 ethylene glycol repeats, greater than 4 ethylene glycol repeats, greater than 5 ethylene glycol repeats, greater than 6 ethylene glycol repeats, greater than 7 ethylene glycol repeats, greater than 8 ethylene glycol repeats, greater than 9 ethylene glycol repeats, or greater than 10 ethylene glycol repeats.

Tn some embodiments, the PEG moiety is of formula (a) or (b):

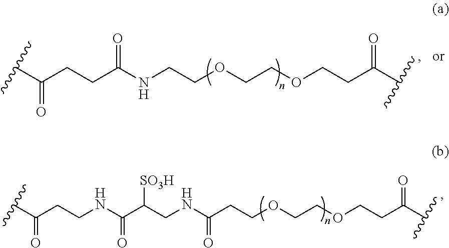

wherein: n is 2 to 20.

In some embodiments, n is 4 to 20, 5 to 20, 6 to 20, 7 to 20, 8 to 20, 9 to 20, or 10 to 20. In some embodiments, n is greater than 2, greater than 3, greater than 4, greater than 5, greater than 6, greater than 7, greater than 8, greater than 9, or greater than 10. In some embodiments, the PEG moiety is of formula (a) and n is 11. In some embodiments, the PEG moiety is of formula (b) and n is 3.

### D. Synthesis of Conjugates

Also provided herein are methods of synthesizing the conjugates. The disclosed methods take advantage of an enzymatic conjugation that can provide site-specific conjugation of the drug. For example, the method can include reacting a sortase with a nanobody portion. The nanobody portion can include at least one nanobody having a sortase recognition site. An example sortase recognition site is LPXT (SEQ ID NO: 1), wherein X is any amino acid. In some embodiments, the sortase recognition site includes an amino acid sequence selected from the group consisting of SEQ ID NO: 1, SEQ ID NO: 2, SEQ ID NO: 3, and SEQ ID NO: 4. Accordingly, sortase can react with a nanobody having a sortase recognition site to form a sortase-nanobody reagent, wherein the sortase-nanobody reagent includes the nanobody portion, a nanobody linking moiety attached to the nanobody portion, the nanobody linking moiety including an amino acid sequence of LPXT (SEQ ID NO: 1), wherein X is any amino acid, and a sortase moiety attached to the nanobody linking moiety.

Any suitable nanobody can be included in the nanobody portion. Description of suitable nanobodies can be found herein. In some embodiments, the at least one nanobody includes a first nanobody, wherein the first nanobody is capable of specifically binding albumin, CD63, EGFR, HER2, MMR, CD16, CTLA-4, CD33, PD-L1, B7-H3, fibronectin, or CD19. In some embodiments, the first nanobody is capable of specifically binding albumin (e.g., albumin-binding nanobody). The at least one nanobody can also include a second nanobody. For example, the nanobody portion can include a first nanobody and a second nanobody. The second nanobody can be capable of specifically binding an immune checkpoint ligand. In some embodiments, the immune checkpoint ligand is 72 CTLA-4, PD-1, PD-L1, B7-H3, B7-H4, HVEM, GITRL, CD80/86, CD155, PD-L1, Galectin 9, LAG3, TIM3, VISTA, TIGIT, PD1, or GITR.

In some embodiments, the sortase is sortase A (SrtA) or an engineered variant thereof. SrtA may be any SrtA, such as Staphylococcus aureus SrtA. SrtA may be from a Gram-positive bacterium, such as, for example, bacteria in a genus selected from Staphylococcus, Streptococcus, Enterococcus, Bacillus, Corynebacterium, Nocardia, Clostridium, Actinobacteria, and Listeria. In some embodiments, SrtA is from S. aureus. The SrtA may be wild-type SrtA or a variant (e.g., engineered) thereof.

The method can further include reacting the sortase-nanobody reagent with a reactive amine reagent. The reactive amine reagent can include a first reactive group. The reaction between the sortase-nanobody reagent and the reactive amine reagent can form a reactive nanobody reagent, where the reactive nanobody reagent includes the nanobody portion, the nanobody linking moiety, and a first reactive group attached to the nanobody linking moiety.

In some embodiments, the reactive amine reagent is of formula (I):

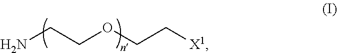

wherein: X¹ is the first reactive group and n′ is 2 to 20.

Tn some embodiments, n′ is 2 to 4, 2 to 6, 2 to 10, 3 to 20, 4 to 20, 5 to 20, 6 to 20, 7 to 20, 8 to 20, 9 to 20, or 10 to 20. In some embodiments, n′ is greater than 2, greater than 3, greater than 4, greater than 5, greater than 6, greater than 7, greater than 8, greater than 9, or greater than 10.

In some embodiments, the reactive nanobody reagent is of formula (II):

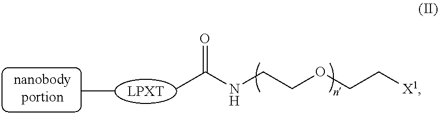

wherein: X¹ is the first reactive group, X is any amino acid, and n′ is 2 to 20. The description for n′ of the reactive amine reagent can also be applied to the reactive nanobody reagent.

The method can also include coupling the reactive nanobody reagent with a reactive drug reagent. The reactive drug reagent can include a drug and a second reactive group. The reaction between the reactive nanobody reagent and the reactive drug reagent can form a conjugate, the conjugate including the nanobody portion attached to the drug by a linker, the linker comprising the nanobody linking moiety.

In some embodiments, the reactive drug reagent is of formula (III-a) or (III-b):

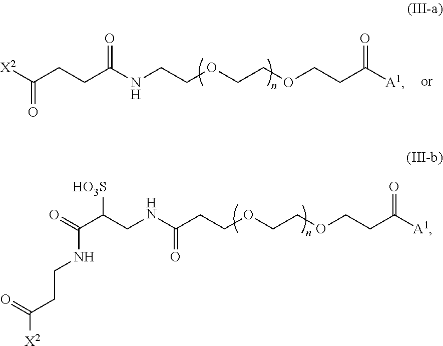

wherein: A¹ is the drug, X² is the second reactive group, and n is 2 to 20. The description for n of the linker herein can also be applied to the reactive drug reagent.

In some embodiments, the reactive drug reagent is of formula (III-a) and n is 11. In some embodiments, the reactive drug reagent is of formula (II-b) and n is 3.

The first reactive group and the second reactive group can include functional groups that are complimentary to each other in that they can form a covalent bond between the functional groups under appropriate conditions. Representative complimentary functional groups that can form a covalent bond include, but are not limited to, an amine and an activated ester, an amine and an isocyanate, an amine and an isothiocyanate, an amine and a carbonate, thiols for formation of disulfides, an aldehyde and amine for enamine formation, and an azide for formation of an amide via a Staudinger ligation. Functional groups suitable for conjugation also include bioorthogonal functional groups. Bioorthogonal functional groups can selectively react with a complementary bioorthogonal functional group through, e.g., Click-chemistry reactions. Bioorthogonal functional groups include, but are not limited to, an azide and alkyne for formation of a triazole, an azide with BCN and DBCO (e.g., strain promoted azide-alkyne cycloaddition), trans-cyclooctene (TCO) and tetrazine (Tz) (e.g., 1,2,4,5-tetrazine), thiols with maleimide groups via Michael addition, and others.

Tn some embodiments, the first reactive group (e.g., X¹) includes an azide, an alkyne, an alkene, a 1,2,4,5-tetrazine, or a thiol. In some embodiments, the first reactive group includes an azide.

In some embodiments, the second reactive group (e.g., X²) includes an alkyne, an azide, an alkene, a 1,2,4,5-tetrazine, or a thiol. In some embodiments, the second reactive group includes an alkyne. In some embodiments, the second reactive group is

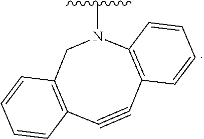 [SMILES: *N1Cc2ccccc2C#Cc2ccccc21]

The drug (e.g., A¹) can be any suitable drug as disclosed herein. In some embodiments, the drug is diABZI as disclosed herein. By using site-specific conjugation techniques disclosed herein, the conjugate can include the drug in a precise manner. For example, the conjugate can include the drug and the nanobody portion at a 1:1 molecular ratio (e.g., 1 drug molecule:1 nanobody portion).

The description of the conjugate, nanobody portion, drug, and linker above may be applied to the disclosed methods of synthesizing the conjugate.

### E. Example Conjugates

In some embodiments, the conjugate includes a nanobody portion, the nanobody portion including an albumin-binding nanobody capable of specifically binding albumin and a second nanobody capable of specifically binding an immune checkpoint ligand; a drug that includes a STING agonist; and a linker including a nanobody linking moiety attached to the nanobody portion and a hydrophilic moiety attaching the nanobody linking moiety to the drug.

In some embodiments, the conjugate includes a nanobody portion, the nanobody portion including an albumin-binding nanobody capable of specifically binding albumin and a second nanobody capable of specifically binding 72 CTLA-4, PD-1, PD-L1, B7-H3, B7-H4, HVEM, GITRL, CD80/86, CD155, PD-L1, Galectin 9, LAG3, TIM3, VISTA, TIGIT, PD1, or GITR; a drug that includes a STING agonist; and a linker including a nanobody linking moiety attached to the nanobody portion and a PEG moiety attaching the nanobody linking moiety to the drug.

In some embodiments, the conjugate includes a nanobody portion, the nanobody portion including an albumin-binding nanobody capable of specifically binding albumin and a second nanobody capable of specifically binding PD-L1 or B7-H3; a drug that includes diABZI; and a linker including a nanobody linking moiety attached to the nanobody portion and a PEG moiety attaching the nanobody linking moiety to the drug.

### 3. Uses of the Conjugates

### A. Pharmaceutical Compositions

Also disclosed herein are pharmaceutical compositions that include the conjugate and a pharmaceutically acceptable excipient. Examples of pharmaceutically acceptable excipients include, but are not limited to, buffering agents (e.g., phosphate buffered saline), carbohydrates (e.g., glucose, trehalose, starch, etc.), and combinations thereof. The description of the conjugate, nanobody portion, drug, and linker above may be applied to the disclosed pharmaceutical compositions.

### B. Administration

The conjugate or pharmaceutical composition thereof can be administered prophylactically or therapeutically. In prophylactic administration, the conjugate or pharmaceutical composition thereof can be administered in an amount sufficient to induce a response. In therapeutic applications, the conjugate or pharmaceutical composition thereof can be administered to a subject in need thereof in an amount sufficient to elicit a therapeutic effect. Amounts effective for this use will depend on, e.g., the particular conjugate regimen administered, the manner of administration, the stage and severity of the disease, the general state of health of the patient, and the judgment of the prescribing physician.

The conjugate or pharmaceutical composition thereof can be delivered via a variety of routes to the subject. The conjugate or pharmaceutical composition thereof can be delivered via systemic administration or locally at a site of interest. Example delivery routes include, but are not limited to, intravenous and locally at the site of injury (e.g., site associated with a cancer). In some embodiments, the conjugate or pharmaceutical composition thereof is administered intravenously or locally at a site of interest.

The conjugate or pharmaceutical composition thereof may be presented in a single dose or as divided doses administered at appropriate intervals, for example, as two, three, four or more sub-doses per day. The sub-dose itself may be further divided, e.g., into a number of discrete loosely spaced administrations.

As will be readily apparent to one skilled in the art, the useful in vivo dosage to be administered and the particular mode of administration will vary depending upon the age, weight, the severity of the affliction, and subjects treated, the particular conjugates and/or compounds employed, and the specific use for which these compounds are employed. The determination of effective dosage levels, that is the dosage levels necessary to achieve the desired result, can be accomplished by one skilled in the art using routine methods, for example, human clinical trials, in vivo studies and in vitro studies.

Dosage amount and interval may be adjusted individually to provide plasma levels of the biologically active agent which are sufficient to maintain the modulating effects, or minimal effective concentration (MEC). The MEC will vary for each agent but can be estimated from in vivo and/or in vitro data. Dosages necessary to achieve the MEC will depend on individual characteristics and route of administration. However, assays well known to those in the art can be used to determine plasma concentrations. Dosage intervals can also be determined using MEC value. Pharmaceutical compositions can be administered using a regimen which maintains plasma levels above the MEC for 10-90/a of the time, such as between 30-90% or between 50-90%. In cases of local administration or selective uptake, the effective local concentration of the conjugate may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the symptoms to be treated and the route of administration. Further, the dose, and perhaps dose frequency, will also vary according to the age, body weight, and response of the individual patient. A program comparable to that discussed above may be used in veterinary medicine.

### C. Methods of Treating a Disease or a Disorder

Disclosed herein are methods of treating a disease or a disorder in a subject (e.g., in need thereof). The method can include administering to the subject an effective amount of one or more of the disclosed conjugates. The conjugate can be administered optionally with a pharmaceutically acceptable excipient as disclosed herein.

Example diseases include, but are not limited to, a cancer, a viral infection, and multiple sclerosis. In some embodiments, the disease or disorder is cancer. Example cancers include, but are not limited to, melanoma, breast cancer, neuroblastoma, renal cell carcinoma, colon cancer, lung cancer, glioma, glioblastoma, and pancreatic cancer. In some embodiments, the disease or disorder is breast cancer or melanoma.

The method can lead to advantageous benefits. For example, the method can increase serum levels of a cytokine in the subject. Example cytokines include, but are not limited to, interferon β (IFN-β), TNFa, CXCL 10, and CXCL1. In addition, in embodiments where the disease or disorder is a cancer, the method can reduce tumor growth, improve survival, or both.

The description of the conjugate, nanobody portion, drug, and linker above may be applied to the disclosed methods of treating a disease or a disorder.

### 4. Examples

### Abbreviations

DIPEA is N,N-Diisopropylethylamine or Hunig's base;
BuOH is n-butyl alcohol;
Na₂S₂O₄ is sodium dithionite;
MeOH is methanol,
EtOAc is ethyl acetate;
DCM is dichloromethane;
DMF is dimethylformamide;
DMSO is dimethyl sulfoxide;
EDC is 1-ethyl-3-(3-dimethylaminopropyl)carbodiimide);
TEA is triethylamine;
TFA is trifluoroacetic acid;
NHS is N-hydroxysuccinimide;
Rr is retention factor;
TLC is thin-layer chromatography;
HRMS is high-resolution mass spectroscopy;
NMR is nuclear magnetic resonance;
atm is atmospheric pressure;
eq. or equiv. is equivalents;
rt or RT is room temperature;
hr or h is hour; and
min or mins is minutes.

### A. Materials and Methods

Cell Lines and Materials. All chemicals involved in synthesis of target compounds were reagent grade unless stated otherwise. DNase, isopropyl thiogalactoside (IPTG), and dimethyl sulfoxide (DMSO) were purchased from Sigma-Aldrich. Azido-PEG3-Amine and DBCO-PEG12-NHS Ester were purchased from Broadpharm. Magnesium sulfate, sodium hydroxide, sodium azide, sodium acetate, sodium azide, sodium chloride, sodium bicarbonate, sodium hydroxide, 2xYT media, kanamycin, Nickel NTA resin, and all other organic solvents were purchased from Thermo Fisher Scientific. All DNA block segments involved in cloning protein inserts were purchased from Integrated DNA Technologies (IDT) with standard desalting as means of purification. A 44 pET28-b(+) expression vector, Q5 Hot Start Master Mix 2x, T4 DNA ligase, Golden Gate Master 45 Mix (BsaI-HF v2), DH5α E. coli, and T7 Shuffle Express were used. E. coli chemically competent cells were purchased from New England Biolabs (NEB). Qiaprep Miniprep Spin kits were purchased from Qiagen. THP1-Dual and A549-Dual cell lines were purchased from InvivoGen. A549-Dual cells were cultured in Dulbecco's Modified Eagle Medium (DMEM; Gibco) and was supplemented with 2 mM L-glutamine, 4.5 g/L glucose, 10% heat-inactivated fetal bovine serum (HI-FBS; Gibco), a mixture of 100 U·mL⁻¹ penicillin/100 μg·mL~¹ streptomycin (Gibco), as well as 100 μg/mL Normocin. THP1-Dual cells were cultured in Roswell Park Memorial Institute (RPMI) 1640 Medium (Gibco) and was supplemented with 2 mM L-glutamine, 4.5 g/L glucose, 10%/6 heat inactivated fetal bovine serum (HI-FBS; Gibco), a mixture of 100 U·mL~¹ penicillin/100 μg mL⁻¹ streptomycin (Gibco), as well as 100 μg/mL Normocin. Every other passage, both Blasticidin (InvivoGen) and Zeocin (InvivoGen) were added at a concentration of 200 μg/mL. The murine breast cancer cell line EMT6 and melanoma cell lines B16.F10 and B16.F10-LUC2 were purchased from American Type Culture Collection (ATCC), where EMT6 cells were grown in RPMI supplemented with 2 mM L-glutamine, 4.5 g/L glucose, 10% HI-FBS, and 100 U ml−1 penicillin/100 μg·mL⁻¹ streptomycin. B16.F10 and B16.F10-LUC2 cells were cultured in DMEM supplemented with 2 mM L-glutamine, 4.5 g/L glucose, 10% heat-inactivated fetal bovine serum, and 100 U ml-1 penicillin/100 g mL⁻¹ streptomycin. B16.F10-OVA cells were cultured in DMEM supplemented with 2 mM L-glutamine, 4.5 g/L glucose, 10% heat-inactivated fetal bovine serum, and 100 U·mL⁻¹ penicillin/100 μg·mL⁻¹ streptomycin with continuous selection using Geneticin (G418; Gibco) after every cell passage at a concentration of 500 μg/mL. All cell types used in the study were grown in a humidified atmosphere at 37° C. in 5% CO₂.

Cloning of Proteins. Gene cassette was purchased from IDT 68 in the form of a gene block, with cloning restriction sites placed on both flanking regions (BsaI—GGTCTC). In the case of a fusion protein, a genetic sequence was placed between the two domains (XTEN-SGSETPGTSESA (SEQ ID NO: 84)). For sortase mediated bioconjugation of nanobodies, a C-terminal sequence was incorporated (LPETGGHHHHHHEPEA (SEQ ID NO. 4)). The gene fragment was digested with BsaI-HF v2 in a golden gate master mix (New England Biolabs) and ligated into a pET28-b(+) plasmid. The construct was transformed into chemically competent DH5α (New England Biolabs) E. coli and plated on LB agar with Kanamycin. The sequence-verified nanobody was transformed in pET28b into T7 Shuffle Express (New England Biolabs) with E. coli as the expression strain.

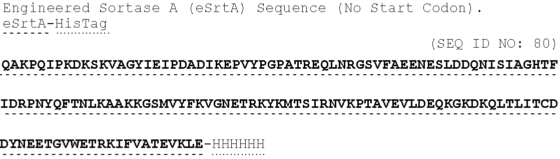

Formula: C₇₈₅H₁₂₃₄N₂₂₀O₂₄₂S₃
Molecular Weight: 17721.94 Da
ε₂₈₀ = 14440 M⁻¹ cm⁻¹

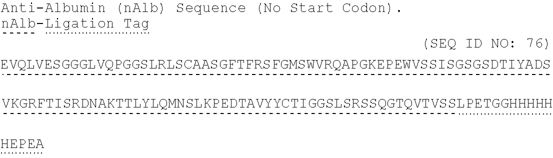

Formula: C₆₀₆H₉₃₉N₁₇₅O₁₉₈S₄
Molecular Weight: 13972.42 Da
ε₂₈₀ = 17085 M⁻¹ cm⁻¹

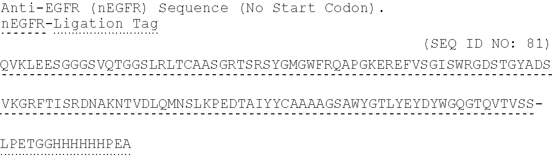

Formula: C₆₆₂H₉₉₆N₁₉₄O₂₁₄S₄
Molecular Weight: 15224.60 Da
ε₂₈₀ = 34045 M⁻¹ cm⁻¹

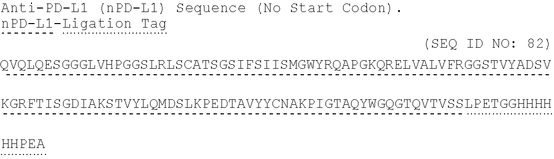

Formula: C₆₂₅H₉₆₆N₁₇₈O₁₉₂S₄
Molecular Weight: 14173.86 Da
ε₂₈₀ = 20065 M⁻¹ cm⁻¹

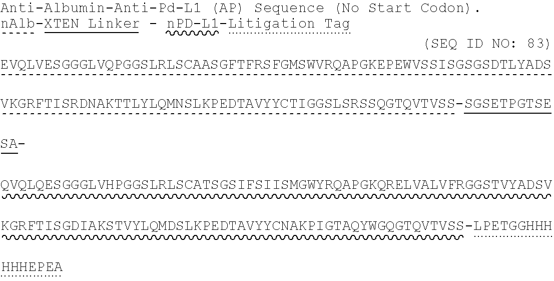

Formula: C₁₁₉₅H₁₈₆₃N₃₃₇O₃₈₈S₈
Molecular Weight: 27415.44 Da
ε₂₈₀ = 37150 M⁻¹ cm⁻¹

Expression and Purification of Proteins. 5 μL of Kanamycin (stocked at 50 mg/mL) was added to a culture tube containing 5 mL 2xYT media and inoculated with a stab of protein (cloned into a NEB T7 Shuffle Express cell line). The culture was incubated at 30° C., with shaking at 250 RPM, for 16 hours. Each culture was transferred to a 2 L baffled flask containing 500 mL of 2xYT media and 500 μL of Kanamycin (25 mg) and shaken at 30° C. in an innova 42R (New Brunswick Scientific) incubator for 4.5-5 hours (until the OD600 reached ~0.8). The cultures were then cooled to −16° C. and induced with IPTG (2.5 mM final concentration). The induced cultures were shaken overnight (20-24 hours) at 16° C. The bacteria were harvested the next day by centrifugation (3900 rpm for 10 min) and the pellet was reconstituted in 1× PBS with Dnase with a tablet of protease inhibitor cocktail (EDTA free). The cells were lysed by sonication on an ice bath in 5 second increments over 10 minutes. The resulting bacterial lysate was centrifuged (11000 rpm for 20 min) to remove cellular debris. The lysate was added to a 50 mL Kontes Flex column (Kimbal Kontes Glassware) containing 3 mL of Nickel NTA histidine binding resin that was preequilibrated with 1× PBS buffer. This column was placed on a rotating shaker at room temperature for 1-2 hrs. After this period, the supernatant was drained from the column using gravity and the column washed with 1× PBS buffer twice. Weakly bound proteins were first washed off of the resin using a low concentration elution buffer (2× 10 mL, 10 mM imidazole, 0.02% NaN₃, 1× PBS pH 7.4 at 25° C.). The bound protein was then eluted from the resin using elution buffer (15 mL, 150 mM imidazole, 0.02% NaN₃, 1× PBS pH 7.4 at 25° C.). The eluate was then concentrated to 0.5 mL in a 15 mL Microcon 10 kDa Centrifugal Filter Unit (Millipore) and subsequently purified by size exclusion chromatography (SEC) via an Akta FPLC (Cytiva), on a Hi-Load 16/60 Superdex 200 column using 1× PBS and 0.02% NaN₃, pH 7.4 at 4° C. as the running buffer. Pure fractions were determined by SDS-PAGE, pooled together with buffer exchange to 1× PBS not containing NaN₃, and stocked at either −20° C. or 4° C.

Enzymatic Bioconjugation and Click Chemistry Reactions. Bioconjugation reactions occurred in mild conditions (20 mM HEPES at pH 7.4, 150 mM NaCl, and 10 mM CaCl₂)) between eSrtA (100 μM) and a nanobody containing a C-terminal ligation tag (75 μM) using a primary amine containing functional group (20 mM). Reactions occurred with mixing by a rotary shaker overnight (16 h) and were quenched by the addition of a 1:1 volume of a chelating agent EDTA containing solution (20 mM HEPES at pH 7.4, 300 mM NaCl, and 10 mM EDTA) for one hour. After the reaction was stopped, the solution was concentrated, and buffer exchanged to 1× PBS (without NaCl or MgCl₂) three times by centrifugal dialysis. The protein solution was then immobilized to Nickel NTA histidine binding resin over 2 hours, and unbound protein was collected by washing the resin with 1× PBS. For nanobodies that contain a histidine in the native sequence, proteins were eluted in mild conditions (10 mM imidazole in 1× PBS). Collected protein was concentrated and buffer exchanged to 1× PBS by centrifugal dialysis and verified by ESI-MS and SDS-PAGE. Click chemistry reactions proceeded by the addition of 5 eq. (molar) of the complementary handle (e.g., if an azide was placed on the nanobody, the click chemistry reaction would proceed with the addition of 5 eq. of DBCO-containing moiety). After 48 hours of reaction between the protein azide and the DBCO-moiety, the mixture was purified by centrifugal dialysis four times, and verified for purity by UV-VIS, ESI-MS, and SDS-PAGE.

Sodium Dodecyl Sulfate-Polyacrylamide Gel Electrophoresis (SDS-PAGE). Protein samples were diluted in 1× PBS to 10 μM before analysis. 10 μL of the protein sample was mixed with 10 μL of reducing Laemmli buffer. Samples were boiled at 95° C. for 5 minutes, 170 μL and 15 μL of each sample was loaded into a 15-well, 4-15% Tris-glycine precast SDS-PAGE (Biorad) and ran at a constant 150 V with 343 mA for 30 minutes. The gel was then either first imaged for fluorescence on a UV-transilluminator or directly stained using Coomassie-B-250.

Electrospray Ionization Liquid Chromatography Mass Spectrometry (ESI-MS). Proteins were buffer exchanged into ammonium acetate (pH 5.5) and concentrated to approximately 100 μM. ESI-MS data were collected using an Agilent 6210A time-of-flight (TOF) mass spectrometer at a range of 50-20,000 m/z over a period of two minutes. Data were analyzed with Agilent MassHunter IM-MS Acquisition Data software to reveal m/z data, where files were condensed across the two-minute run. These m-z data were deconvoluted using a maximum entropy deconvolution calculation using UniDec to give the deconvoluted mass spectra using background subtraction between a range of 1,000-5,000 m/z and with an export range of 5,000-50,000 Da.

Computational Modeling and Analysis of nAlb Nanobody. nAlb was modeled in silico using RoseTTAFold (GitHub; RosettaCommons) and binding between 1 (PDB: 1A06) and nAlb was predicted using RosettaDock through ROSIE (Rosetta Online Server that Includes Everyone; Pittsburgh). After an initial screening for best fits of the docking between I (receptor) and nAlb(ligand), the best fit model was then returned for rescreening to confirm an optimal energy conformation between the structures. The final structures of nAlb and the bound nAlb-I complex were exported to PyMOL for generating a figure of the structure.

Synthesis and NMR Verification of DBCO-PEG11-diABZL Synthesis of the DBCO conjugated STING agonist (diABZI) is shown in FIG. 19, with NMR verification in FIGS. 20A-20B. First, a STING agonist was generated that features a reactive amine handle, which was synthesized in four steps. Briefly, aryl amination of an aryl chloride 1 with an amine 2 gave a di-nitro analog, compound 3. The di-nitro compound 3 was subjected to reduction using sodium dithionite in methanol, generating a di-amine moiety 4. Compound 4 was then treated with isothiocyanate, followed by EDC coupling, to reveal a boc-protected analog, compound 5. Next, the boc-group from compound 5 was deprotected by treating with TFA:DCM. To a stirred solution of amine 6 (100 mg, 0.089 mmol, 1 eq.) in 5 mL DMF was added Hunig's base (77 μL, 0.44 mmol, 5 eq.) under argon atmosphere, at room temperature. After stirring for 5 min, a solution of activated NHS ester (98 mg, 0.098 mmol, 1.1 eq.) in DMF (5 mL) was added dropwise and stirred overnight (16 h). The solvent was evaporated to get crude product 7, which was purified by silica gel column chromatography using a mixture of methanol/dichloromethane as an eluent (5% to 25% MeOH) to get the desired product as a solid (70 mg, 0.042 mmol, yield 43%). (R_{f}=0.5 in 20% MeOH in DCM). 1H NMR (400 MHz, DMSO) δ 8.01-7.93 (m, 2H), 7.88 (t, J=5.7 Hz, 1H), 7.75 (t, J=5.7 Hz, 1H), 7.67-7.60 (m, 4H), 7.49-7.42 (m, 3H), 7.38-7.27 (m, 7H), 6.49 (d, J=7.1 Hz, 2H), 5.88-5.79 (m, 2H), 5.01 (d, J=13.9 Hz, 1H), 4.91 (dd, J=29.6, 4.2 Hz, 4H), 4.53-4.49 (m, 4H), 3.98 (t, J=6.0 Hz, 2H), 3.72 (s, 3H), 3.60-3.57 (m, 2H), 3.54 (t, J=6.5 Hz, 2H), 3.47 (broads, 46H), 3.30-3.26 (m, 2H), 3.14-3.05 (m, 4H), 2.26 (t, J =6.5 Hz, 2H), 2.09 (s, 3H), 2.08 (s, 3H), 2.01-1.96 (m, 1H), 1.78-1.72 (m, 1H), 1.68 (p, J=6.5 Hz, 2H), 1.28-1.27 (m, 6H). 13C NMR (151 MHz, DMSO) δ 171.57, 171.50, 170.52, 168.06, 167.33, 152.50, 152.45, 152.06, 148.88, 145.50, 145.28, 144.65, 140.37, 140.33, 132.87, 130.54, 130.49, 130.07, 129.37, 128.62, 128.58, 128.44, 128.25, 128.13, 127.24, 125.60, 122.99, 121.86, 120.11, 120.04, 114.67, 109.72, 108.61, 106.00, 105.83, 105.58, 70.21, 70.14, 70.10, 70.00, 69.94, 69.45, 67.26, 56.45, 55.34, 53.85, 46.05, 45.05, 42.12, 38.95, 36.57, 35.61, 30.80, 30.17, 29.13, 18.46, 17.17, 16.58, 13.57, 12.74. HRMS (ESMS) Calculated for C₈₄H₁₁₁N₁₅O₂₁ [M+Na]⁺: 1688.7977, found 1688.7982.

In Vitro Reporter Cell Assays. Cell reporter assays were utilized in THP1-Dual and A549-Dual cell lines, as adapter from the manufactures protocol. Briefly, cells were plated at a density of 50,000 cells/well in a total volume of 180 μL of supplemented media in cell-culture treated 96-well plates overnight. The following day, cells were dosed with 20 μL of treatment groups (for a total volume of 200 μL/well in a 10:1 dilution, with either a 1:1 or 2:1 dilution down the plate) overnight. After 24 h of treatment, cells were pelleted at 1500 RPM for 5 minutes in the centrifuge, and μL of supernatant was taken and plated in a white-walled 96-well plate for analysis by QUANTI Luc™ (InvivoGen) assay. After loading in a plate reader, 50 μL of QUANTI-Luc reagent was added to each well and luminescence was measured for determination of cell-based activity. To the remaining cells in the cell-culture treated 96-well plate were added 30 μL of Cell-Titer Glo reagent (Promega) and the plate was incubated at 37° C. for 1 h. After incubation, the plate was loaded into the plate reader and luminescence was measured to determine cell-mediated toxicity. Data were recorded in triplicate and analyzed in GraphPad PRISM (Version 10), with data reported with standard error of the mean (SEM).

In Vitro BMDC/BMDM Maturation and Activity. Bone marrow primary cells were harvested from both the femur and tibia of female C57BL/6 mice, aged between 6-8 weeks. After harvesting, cells were flushed with cold 1× PBS, centrifuged at 1500 rpm for 5 min, and resuspended in complete media (RPMI 1640 supplemented with 10% HI-FBS (Gibvo), 100 U·mL⁻¹ penicillin/100 μg·mL⁻¹ streptomycin (Gibco), 2 mM L-glutamine, 10 mM HEPES, 1 mM sodium pyruvate, 1× non-essential amino acids, and 50 μM β-mercaptoethanol. 20 ng/mL GM-CSF was added to culture BMDCs, and 20 ng/mL of M-CSF was added to culture BMDMs. A single cell suspension was generated by passing the collected cells through a 70 μm sterile cell strainer (Fisherbrand™; Thermo Fisher Scientific) and cells were then plated in non-tissue-culture-treated petri dishes (REF 351029; Corning) and incubated at 37° C. with 5% CO₂. Cells were provided with fresh culture media—supplemented with growth factors—on days 3, 5, and 7. On day 8, the cells were collected and confirmed for either CD11c⁺ expression (BMDCs) or CD11b⁺F4/80⁺ expression (BMDM) using flow cytometry with fluorescent anti-CD11c⁺ (Clone N418; BioLegend), anti-CD11b (Clone M1/70, BioLegend), and anti-F4/80 (Clone BM8, BioLegend) antibodies. Primary cells were seeded into 12-well plates for analysis by qPCR or 96-well plates for in vitro flow cytometry.

Quantitative RT-PCR (qPCR). RNA was extracted either from animal tissue (by TissueLyser II, Qiagen) or from in vitro cell cultures using the RNeasyR Plus Mini Kit (Qiagen) according to the manufacturer's protocol. cDNA was generated through a reverse transcriptase reaction using the iScript cDNA synthesis kit (Bio-Rad), by following the manufacturer's instructions. To run the qPCR, cDNA was mixed with TaqMan gene expression kits (primer and master mix) to a final volume of 20 μL and run on the Bio-Rad CFX Connect Real-time System, with a threshold cycle number determination made by the Bio-Rad CFX manager software V.3.0. Primers used included; mouse Ifib1 (Mm00439552 s1), mouse Inf(Mm00443258_ml), mouse CxcI10 (Mm00445235_ml), mouse Cxcl1 (Mm04207460_ml), and mouse Hmbs (Mm01143545_ml). Gene expression was first normalized to the housekeeping gene, Hnbs, and then normalized to the PBS treatment within groups using the 2-ddCt analysis method.

Evaluation of Nanobodies in Tumor Models. 6-8-week C57BL/6 mice (The Jackson Laboratory) were inoculated with B16.F10, B16.F10-LUC, or B16.F10-OVA models. For EMT6 models, 6-8 week Balb/C female mice (The Jackson Laboratory) were used. Tumors were generated in B16.F10 and B16.F10-OVA models by subcutaneous injection of 5×105 cancer cells, suspended in 100 μL of PBS, at the right flank side of the mouse. B16.F10-LUC inoculations were performed by intravenous (I.V.) injection at a volume of 100 μL and using 1×106 cancer cells. EMT6 inoculations were orthotopic and placed at the left-side 4^{th} mammary fat pad at a volume of 100 μL and using 5×10 cancer cells. When the volume of tumors reached ~75-100 mm³, mice were treated by I.V. injection of nanobodies or free diABZT compound 3 (using 40% PEG400 as an excipient for free diABZI, Sigma), or intraperitoneal (I.P) injection of commercial anti-PD-L1 IgG (Clone BEO101, Bio X Cell) (100 μL per injection). Tumor volume calculations were calculated using Vₜᵤₘₒᵣ=L×W2×0.5, in which Vₜᵤₘₒᵣ is tumor volume, L is tumor length, and W is tumor width. Tumor volume, total murine mass, and murine well-being were recorded for the duration of the study. The endpoint for maximum tumor volume (i.e. survival) during studies was 1500 mm³.

Adoptive OT-1 T Cell Transfer in B16.F10-OVA Tumor Model. 6-8-week C57BL/6 CD45.1^{+/-} OT-I mice were euthanized with spleens harvested using EasySep Mouse CD8⁺ T Cell Isolation Kit (STEMCELL Technologies). Briefly, T cells were activated in vitro in supplemented RPMI 1640 (Gibco) with 10% HI-FBS (Gibco), 1% penicillin/streptomycin (Gibco), 50 μM β-mercaptoethanol (MilliporeSigma), 1 mM sodium pyruvate, minimum essential medium NEAA (non-essential amino acids) (Gibco), 10 mM HEPES (Gibco), recombinant mouse interleukin-2 (10 U/mL;MilliporeSigma), and Dynabeads Mouse T-Activator CD3/CD28 (at a bead-to-cell ratio of 1:1; Gibco) at 37° C. in a CO₂ incubator (5%). After 5 days, T cells were magnetically separated from Dynabeads and allowed to rest for 24 h before use. The following day, in the B16.F10-OVA model, 5×10⁵ OT-I CD8⁺ T cells were adoptively transferred by retro-orbital injection.

Immunofluorescent analysis of EMT6 Tumors. 5-micron Paraffin-embedded slides were prepared for immunofluorescence and stained with anti-CD31 (cell signaling #77699; 1:500) and anti-CD45 (cell signaling #70257; 1:500). Slides were deparaffined in xylene and rehydrated in serial ethanol dilutions. Antigen retrieval was performed by heating slides for 17 minutes in Tris EDTA buffer, pH 9 in a pressure cooker at 110° C. Slides were cooled to room temperature and then blocked with 2.5% horse serum (vector labs). After blocking, slides were incubated overnight at 4° C. with primary antibody in horse serum. Slides were then incubated in anti-rabbit HRP secondary (vector labs) for 1 h at room temperature the following day and subsequently incubated in 1:500 Opal 520 (green) or Opal 570 (red) (Akoya) for 10 minutes. For serial staining, slides were stripped using Citric Acid buffer, pH 6.1 in a pressure cooker at 110° C. for 2 minutes and then staining was repeated using different antibody and Opal fluorophore. After last Opal staining, slides were mounted using antifade gold mount with DAPI (Invitrogen). Stained images were acquired using a Keyence digital microscope system. Images were analyzed with Fiji software. Quantification of markers was done by measuring total amount of fluorescence divided by total number of cells (DAPT).

Flow Cytometric Experiments and Analysis. EMT6 tumor bearing Balb/c and B16.F10-OVA bearing C57BL/6 mice were euthanized either 24 h or 48 h after final treatment. Spleens and tumors were harvested, weighed, and placed onto ice. Tumors were digested in RPMI 1640 media containing a tumor dissociation kit (collagenase III and deoxyribonuclease 1, Miltenyi Biotech). Tumors were dissociated using an OctoMACS separator (Miltenyi Biotech) and incubated for 30 min at 37° C. for complete digestion. Tumors and spleens were mashed and separated into single cell suspensions using a 70 μm cell strainer (Fisherbrand™; Thermo Fisher Scientific) and red blood cells were used twice using ACK lysis buffer (Gibco). Cells were resuspended in flow buffer (1× PBS supplemented with 2% FBS and 50 M dasatinib), counted, and stained with Fc-block (aCD16/32, 2.4G2, Tonbo) for 15 min at 4° C., and then stained with the appropriate antibodies for 1 hr at 4° C. (found below and in in Tables 1-2). After staining, cells were then washed again with FACS buffer, fixed with 2% paraformaldehyde for 10 min, washed again with FACS buffer containing AccuCheck counting beads, and analyzed on a Cytek Aurora flow cytometer. All flow cytometry data were analyzed using FlowJo software (version 10. Tree Star). Representative flow cytometry piots and gating schemes are shown in FIGS. 31A-31B, FIGS. 32A-32B, FIGS. 33A-33B, and FIG. 34.

TABLE 1
Cy5 Uptake Flow Cytometry Panel (Balb/c).

| Marker | Fluorochrome | dilution | Company | Catalog |
|---|---|---|---|---|
| CD206 | BV605 | 100 | Biolegend | 141721 |
| CD3 | BV510 | 40 | Biolegend | 100353 |
| CD4 | APC/Fire810 | 200 | Biolegend | 100480 |
| CD8 | Pacific blue | 100 | Biolegend | 100725 |
| B220 | PE/CY5 | 200 | Biolegend | 103210 |
| NKp46(BALB/C) | PE/CY7 | 100 | Biolegend | 137618 |
| FOXP3 | PerCP-eFluor ™ 710 | 100 | eBiosciences/Invitrogen | 46-5773-82 |
| CD69 | FITC | 100 | Biolegend | 104506 |
| PD-1 | PE-DAZZLE 594 | 100 | Biolegend | 135228 |
| KI67 | AF532 | 100 | eBiosciences/Invitrogen | 58-5698-82 |
| CD11B | BV750 | 200 | Biolegend | 101267 |
| CD11C | AF700 | 100 | Biolegend | 117320 |
| LY6G | PERCP | 100 | Biolegend | 127654 |
| LY6C | BV421 | 200 | Biolegend | 128032 |
| I-A/I-E(MHC II) | BV711 | 100 | Biolegend | 107643 |
| F4/80 | AF488 | 100 | Biolegend | 123120 |
| CD31 | PE | 100 | Biolegend | 102507 |
| CD45.2 | BV785 | 100 | Biolegend | 109839 |
| PD-L1 | BV480 | 100 | BD | 748275 |
| Viability | Eflour780 | 20000 | Invitrogen | 740614 |
| CD86 | BV650 | 100 | Biolegend | 105036 |

TABLE 2
Cell Population and Immunophenotyping Flow Cytometry 804 Panel (Balb/c).

| Marker | Fluorochrome | dilution | Company | Catalog |
|---|---|---|---|---|
| CD206 | BV605 | 100 | Biolegend | 141721 |
| CD3 | BV510 | 40 | Biolegend | 100353 |
| CD4 | APC/Fire810 | 200 | Biolegend | 100480 |
| CD8 | Pacific blue | 100 | Biolegend | 100725 |
| B220 | PE/CY5 | 200 | Biolegend | 103210 |
| NKp46 | PE/CY7 | 100 | Biolegend | 137618 |
| FOXP3 | PerCP-eFluor ™ 710 | 100 | eBiosciences/Invitrogen | 46-5773-82 |
| CD69 | FITC | 100 | Biolegend | 104506 |
| PD-1 | PE-DAZZLE 594 | 100 | Biolegend | 135228 |
| KI67 | AF532 | 100 | eBiosciences/Invitrogen | 58-5698-82 |
| CD11B | BV750 | 200 | Biolegend | 101267 |
| CD11C | AF700 | 100 | Biolegend | 117320 |
| LY6G | PERCP | 100 | Biolegend | 127654 |
| LY6C | BV421 | 200 | Blolegend | 128032 |
| I-A/I-E(MHC II) | BV711 | 100 | Biolegend | 107643 |
| F4/80 | APC | 100 | Biolegend | 123116 |
| CXCR3 (CD183) | PE | 100 | Biolegend | 126506 |
| CD45.2 | BV785 | 100 | Biolegend | 109839 |
| PD-L1 | BV480 | 100 | BD | 748275 |
| Viability | Eflour780 | 20000 | Invitrogen | 740614 |
| CD86 | BV650 | 100 | Biolegend | 105036 |

Antibodies for Immune Cell Memory in B16.F10-OVA Tumor Model. The antibodies used were eFluor 780 viability dye (eBioscience), anti-CD3e (145-2C11, BV510, BioLegend), anti-CD8a (KT15, FITC, Invitrogen), anti-CD4 (RM4-5, violetFluor™ 450 Anti-Mouse CD4, FisherScientific), andi-CD69 (H1.2F3, PE/Cy7, BioLegend), anti-CD44 (IM7, PE/Cy5, BioLegend), and anti-CD62L (MEL⁻¹⁴, BV711, BioLegend), and PE-labeled pOVA/H-2 Kb tetramer.

Pharmacokinetics and Ex Vivo Imaging Experiments. Healthy (Balb/c or C57BL/6) and EMT6 tumor bearing (Balb/c) mice were injected with 100 μL of Cy5 (either as free dye or as a nanobody conjugate) at a dose of 2 mg/kg intravenously. Blood draws were taken using heparinized capillary tubes (DWK Life Sciences) at discrete time points up to five days after injection. 1 μL of blood was mixed with 50 μL of PBS, centrifuged, and the diluted plasma was collected for analysis. Prescence of Cy5 was determined by fluorescence intensity using a plate reader, with an excitation wavelength of 645 nm and an emission wavelength of 675 nm. Pharmacokinetic analysis was performed in GraphPad Prism (V10) using either a one-phase decay or two-phase decay, in which the reported half-life is the second phase (elimination). Biodistribution studies were performed by excising and weighing hearts, lungs, livers, spleens, kidneys, and tumors. Tissue were washed in 1× PBS and transferred to the stage of the IVIS Lumina Ill (PerkinElmer). Fluorescence (radiant efficiency) was measured with a maximum value of 1.56×10¹⁰, and a minimum of 8.21×10⁸, and areas were drawn for organs to generate average radiant efficiency values (per cm²) using the Living Image software (version 4.5). For B16.F10-LUC studies, lungs were placed in black 12-well plates (Cellvis) and incubated for 5 min in a solution of 1 mg/mL Pierce™ D-Luciferin, Monopotassium Salt (Thermo Fisher Scientific) in 1× PBS. Images were taken on the IVIS and luminescence was quantified as total radiant flux (p/s) for each set of lungs.

Ex Vivo Plasma Analyte Analysis. Blood was collected by either cheek bleed or cardiac puncture in K2EDTA-coated tubes (BD Biosciences). Tubes were centrifuged at 2000×g for 15 min at 4° C., and the plasma was collected for analysis. Cytokine levels were evaluated using either the LEGENDpex™ Mouse Anti-Virus Response Panel (BioLegend) or the LEGENDplex™ Mouse Cytokine Panel 2 (BioLegend), both with V-bottom plates, according to manufacturer's instructions, and data were collected using flow cytometry. Cytokine concentrations were interpolated from standard curves using an asymmetric sigmoidal 5-paramater logistic curve fits (GraphPad Prism V10). Bar plots comparing groups and heat maps of averaged values for groups were generated to analyze results.

NanoString nCounter Analysis of EMT6 Tumors. After three treatments of nAlb-diABZI (1.25 μg, n=3-4), AP-diABZI (1.25 μg, n=3-4), or PBS (n=3-4) in EMT6 bearing female Balb/c mice, tumors were isolated, digested, and 100 ng of RNA was isolated, as described in the qPCR section. RNA were hybridized to the 10360 PanCancer panel, as well as through a selected gene panel, of target-specific fluorescent barcodes and analyzed using NanoString nCounter MAX Analysis system (FIG. 10). The fold change for genes within groups was calculated by comparing against the average normalized gene expression values within PBS treated mice. All statistical significance, and clustering analysis, was performed in R (“The Comprehensive R Archive Network”) based on the genes provided in the 10360 PanCancer panel.

Statistics. All data were plotted, and statistical analysis performed using Prism 10 (GraphPad) software. Unless indicated in the figures, all data are presented as mean t SEM. For comparisons between two groups, unpaired two-tailed Student's t-tests were performed 374 as indicated. For multiple comparisons a one-way ANOVA was performed with post-hoc Tukey's correction for multiple comparisons. For tumor volume, statistically significance was examined through a two-way ANOVA followed by Tukey's adjustment for multiple comparisons. A Log-rank (Mantel-Cox) test was used to compare Kaplan-Meyer survival data.

### B. Results

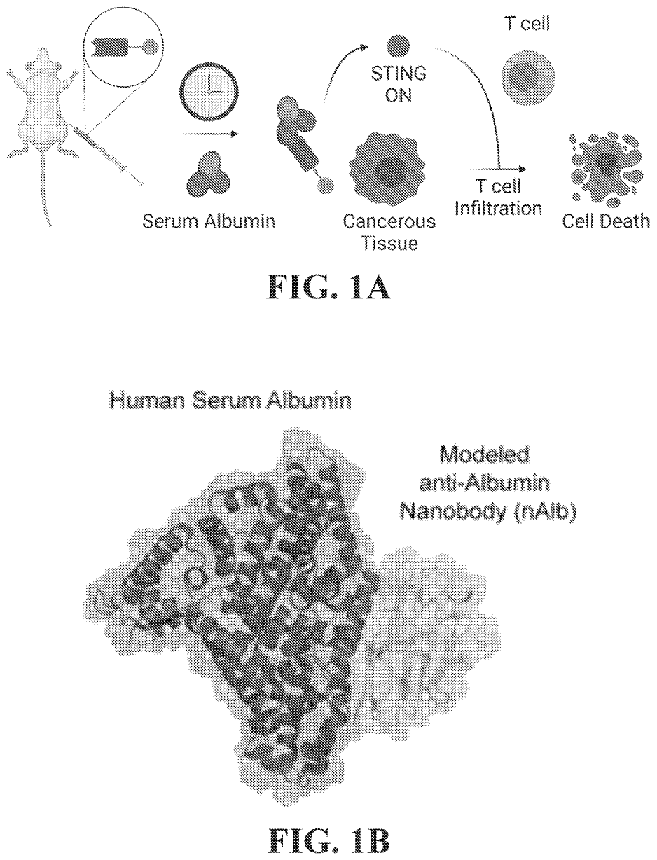

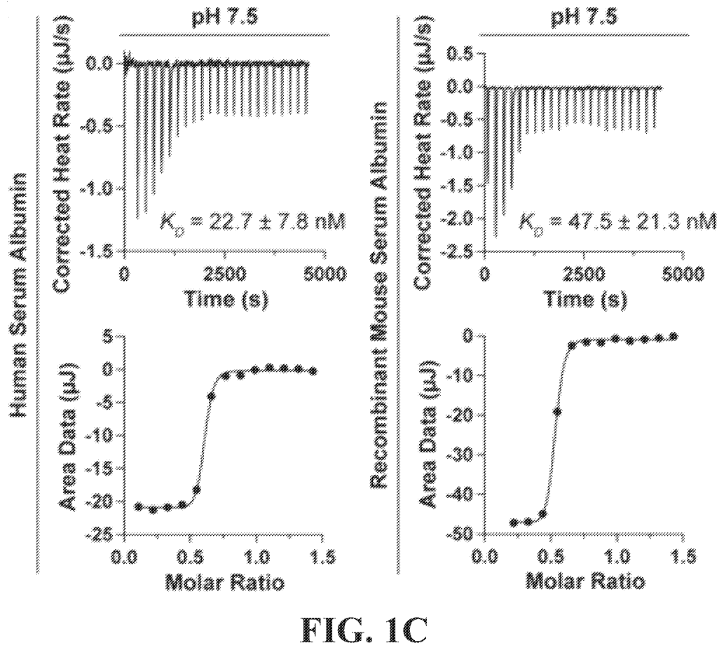

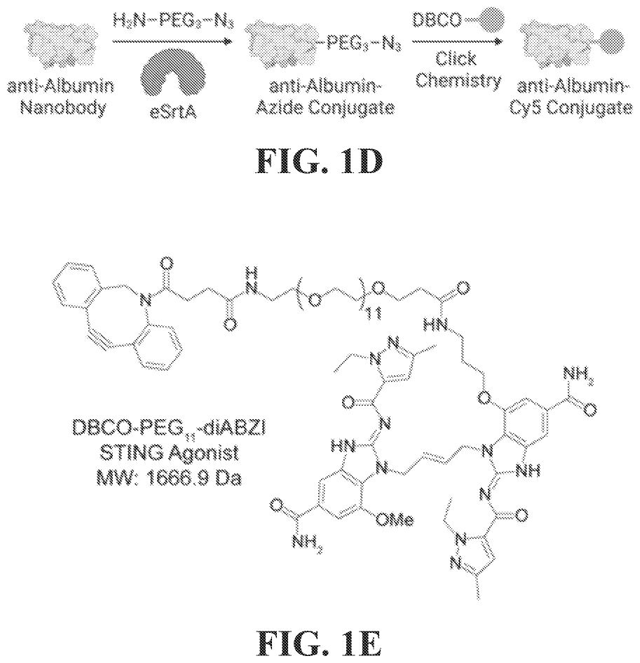

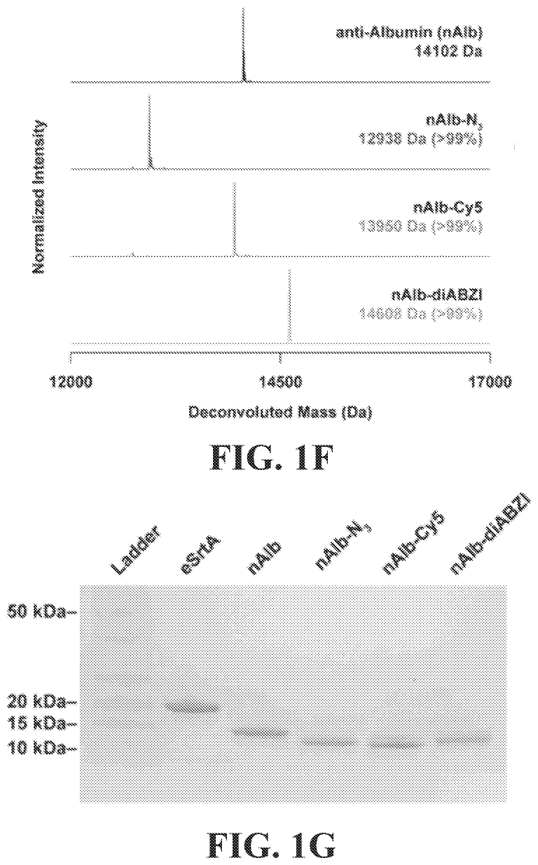

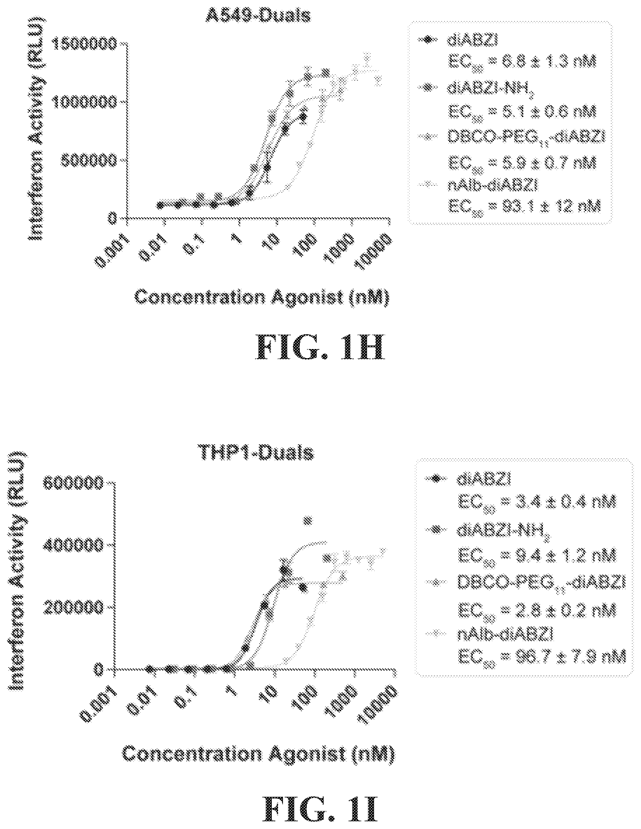

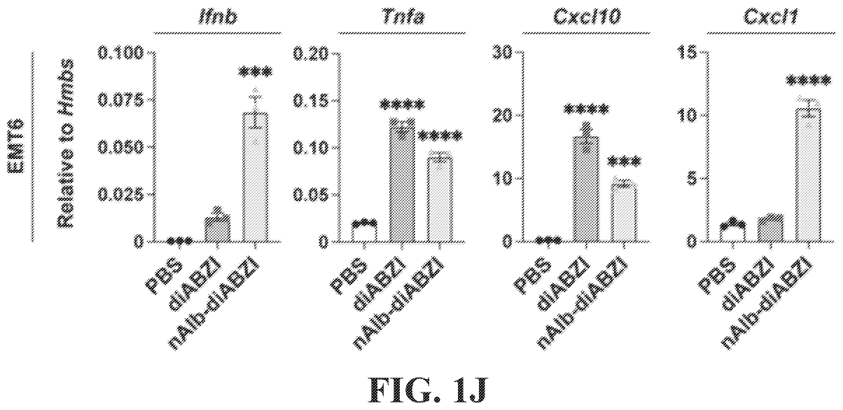

Synthesis of albumin-hitchhiking nanobody-STING agonist conjugates. It was hypothesized that conjugation of a STING agonist to an albumin binding chaperone would extend blood circulation half-life and increase accumulation in cancerous tissue, enriching the production of cytokines and chemokines that facilitated the recruitment, proliferation, and activation of leukocytes to the TME, which promotes cancer cell death (FIG. 1A). While several albumin-binding molecules have been described, the experimental platform described below was built from a nanobody with high affinity for albumin, because nanobodies are highly modular and programmable via genetic engineering, are molecularly well-defined, are amenable to scalable industrial manufacturing, and are components of approved and clinically advanced therapeutics, including ozoralizumab, which contains an anti-albumin nanobody domain. A nanobody domain termed nAlb—that binds with nanomolar affinity to serum albumin was recombinantly expressed (FIG. 1B). The binding of the nanobody domain was modeled to human serum albumin (HSA) using RoseTTAFold, to generate the nAlb nanobody, and RosettaDock to predict the binding site of the nanobody to the serum protein albumin. It was observed that the nAlb nanobody reached an optimal energy conformation through binding at domain IIB of HSA, indicating that nAlb does not compete with albumin binding to FcRn, which facilities its long serum half-life (PDB: 4NOF). The binding affinity of nAlb was verified using isothermal calorimetry (ITC) both at physiological pH (7.5) and at endosomal pH (5.5), where nAlb maintained nanomolar affinity to both HSA and recombinant mouse serum albumin (rMSA) (FIG. 1C and FIGS. 18A-18B). To enable site-selective ligation of STING agonists, the C-terminal of the nAlb nanobody was cloned to present a selective ligation tag (LPETGGHHHHHHEPEA (SEQ ID NO: 4)) that acts as a substrate for an engineered pentamutant of sortase A designed to selectively ligate any primary amine containing small molecule to the C-terminal of a protein, offering high programmability in the design. The ligation of an amino-PEG3-azide linker was demonstrated, which conferred a single azide functional handle on the nAlb nanobody and can be used to ligate cargo via strain-promoted azide-alkyne cycloaddition (FIG. 1D and FIG. 1F). While this strategy is amenable to ligation of diverse classes of STING agonists, a diABZI compound was selected since ongoing clinical trials are exploring similar agents as a systemically administered immunotherapy (e.g., NCT03843359). To enable covalent conjugation to the nanobody, a diABZI variant was synthesized that was functionalized with an azide-reactive DBCO group and a PEGI 1 spacer (DBCO-PEGI 1-diABZI) at the 7 position of one of the benzimidazole groups (FIG. 1E, FIG. 19, and FIGS. 20A-20B), a modification that is not predicted to interfere with diABZI binding to STING. Copper-free click chemistry was then used to install a single DBCO-PEG11-diABZI STING agonist or a DBCO-functionalized Cy5 dye onto the nanobody and verified precise 1:1 conjugation by electrospray ionization mass spectrometry (ESI163MS) (FIG. 1F) and sodium dodecyl sulfate polyacrylamide electrophoresis (SDS-PAGE) (FIG. 1G).

The activity of the nAlb conjugated STING agonist (nAlb-diABZT) was evaluated as well as the parent DBCO-PEGui-diABZT compound and a previously optimized diABZI (compound 3; referred to henceforth as “diABZI”) in two human reporter cell lines for type-I interferon (TFN-I) production: monocytes (THP1-Dual) and lung carcinoma cells (A549-Dual). It was observed that the DBCO-PEG₁₁-diABZI variant retained a near identical EC50 value to the original diABZI agonist from literature, while, as expected, the in vitro activity of the nAlb-diABZI conjugate was reduced but nonetheless maintained high sub-100 nM activity for IFN-I production. Further, the activity of the nAlb-diABZI conjugate in an EMT6 (breast cancer) cell line was tested, demonstrating that nAlb1-diABZI stimulated the expression of STING-driven cytokines Ifnb1, Tnfa, CXCL10, and CXCL1 after 4 hours.

Albumin-hitchhiking nanobodies exhibit tumor tropism and enrich cargo delivery to cancer cells and tumor-associated myeloid cells. It was hypothesized that nAlb conjugates would follow similar mechanisms of cellular internalization as albumin, which primarily uses two receptor-mediated endocytosis pathways for transcytosis, recycling, and cancer cell uptake—SPARC (secreted protein acidic and rich in cysteine) and GP60 (albondin) (FIG. 2A). Following internalization, the nAlb conjugate can either continue to hitchhike onto albumin through recycling or traffic to lysosomal degradation, which was predicted to result in the release of the STING agonist, allowing for binding to STING on the ER membrane. To study nAlb-conjugate internalization, flow cytometry was used to quantify cell surface binding and/or intracellular uptake of nAlb-Cy5 compared to an analogous control anti-EGFR nanobody (nEGFR) that was cloned and Cy5-labeled using the same strategy (FIGS. 21A-21B). Delineation between surface binding and active endocytosis was evaluated at both 37° C. and 4° C. in the THP1-Dual and A549-Dual cells, which are EGFR^{low} and EGFR^{high}, respectively (FIG. 2B). In both cell types, cellular association of nAlb-Cy5 was inhibited at 4° C., demonstrating a dependence on endocytosis. By contrast, nEGFR-Cy5 was able to bind to the surface of EGFR^{high} A549 cells at 4° C., and with increased fluorescence intensity at 37° C. due to cellular uptake. Cellular uptake of nAlb-Cy5 by EMT6 and B16.F10 cancer cells in the absence of albumin (serum free media) was also evaluated, and it was found that the nAlb-Cy5 conjugate was internalized to a greater extent when albumin was present in the media (FIGS. 2C-2D). This mechanism of nAlb-Cy5 internalization via antibody blockade of SPARC and GP60 was further examined (FIG. 2C). In the presence of serum, the addition of anti-SPARC and anti-218 GP60 antibodies to the media decreased the internalization of nAlb-Cy5 in EMT6 cells with a more pronounced effect observed in B16.F10 cells, demonstrating an important role of these two proteins in the endocytosis of nAlb and covalently associated cargo. To understand the pharmacokinetic profile achieved by using anti-albumin nanobody hitchhiking, free DBCO-Cy5, nEGFR-Cy5, and nAlb-Cy5 was intravenously (T.V.) administered in healthy female C57BUJ6 mice and collected blood at discrete time points over several days (FIG. 2E). By measuring the concentration of Cy5 in the serum using fluorescence spectroscopy, the elimination half-life of both the free dye and the nEGFR-Cy5 conjugate was determined to be approximately 5 minutes, matching the expected half-life of a typical nanobody that is rapidly cleared via renal excretion due to its small size (~15 kDa). However, the nAlb-Cy5 conjugate had an elimination half-life of approximately 55 hours, consistent with in situ binding to and hitchhiking on serum albumin, which has a half-life of ~35-40 h in mice. By comparison the half-life of diABZI is ~90 minutes, while that of CDNs is typically <5 min. Next, the biodistribution of DBCO-Cy5, nEGFR-Cy5, and nAlb-Cy5 in female Balb/c mice with orthotopic EMT6 (EGFR*) breast tumors inoculated in the mammary fat pad was tracked. At 24 hours post-administration, mice were euthanized, and major organs and tumors were imaged with an in vivo imaging system (IVIS) instrument to quantify Cy5 fluorescence in tissue (FIG. 2F). Minimal accumulation of Cy5 in major organs for both nEGFR-Cy5 and nAlb-Cy5 conjugates was observed, but significant tumor tropism of only the nAlb-Cy5 conjugate (FIGS. 2G-2H), consistent with the proclivity of albumin to accumulate at tumor sites. Immunofluorescent staining of excised and cryosectioned tumors (FIG. 21) further confirmed nAlb-Cy5 accumulation at the tumor site, with the highest Cy5 fluorescence observed proximal to CD31⁺ tumor vasculature and with Cy5 signal also observed within the tumor stroma (e.g., colocalizing with CD45⁺ immune cells).

Based on the significant tumor accumulation of nAlb-Cy5, next, flow cytometry was used to determine which tumor-associated cell populations internalized the conjugate (FIG. 2J, FIGS. 22A-22B). In parallel, cellular uptake of nAlb-Cy5 in the spleen was evaluated (FIG. 2K), which, while not a major organ of distribution for nAlb-Cy5, is a potentially important secondary lymphoid organ for generating systemic antitumor immunity. At 24 h after I.V. injection of nAlb-Cy5, it was found that ~50% of live cells in the tumor were Cy5⁺, with the majority of cellular uptake of nAlb-Cy5 by CD45⁺CD31⁻ cells, which are primarily breast cancer cells, and tumor-associated CD11b⁺F4/80⁺ macrophages (FIG. 2J), which were the predominant cell populations in the EMT6 tumor model (FIG. 2J, inset) and have been reported to endocytose albumin in tumors. As assessed by Cy5 median fluorescent intensity (MFI), the cell populations with the highest proclivity for nAlb-Cy5 uptake were CD45⁻CD31⁺ endothelial cells, consistent with albumin transport across the endothelial cells, neutrophils, dendritic cells, macrophages, and cancer (CD45⁻CD31⁻) cells (FIGS. 22A-22B). To determine if this cellular uptake profile was influenced by STING activation, nAlb-Cy5 was concurrently administered with nAlb-diABZI and found that the addition of nAlb-diABZI primarily impacted the myeloid cell composition of the tumor at 24 h, resulting in an increased frequency of neutrophils and MDSCs and a reduction in macrophages with minimal changes in the nAlb-Cy5 cellular uptake profile. Consistent with its low overall accumulation in the spleen, only a small percentage (<1%) of splenic MDSCs, macrophages, and B cells internalized nAlb-Cy5 with or without co-administration of nAlb-diABZI (FIG. 2K), further validating nAlb as a carrier for preferential cargo delivery to tumor-associated cell populations.

nAlb-diABZI potently stimulates STING activation in the TME to inhibit tumor growth. Based on the long half-life of nAlb and its capacity to dramatically enrich cargo distribution to tumor sites, next, a dose-response response study was performed to evaluate the therapeutic efficacy of nAlb-diABZI conjugates in an established non- or low-immunogenic (immunologically “cold”) B16.F10 tumor model that is resistant to ICB (FIGS. 23A-23D). Using a treatment regimen that has been employed for evaluation of STING agonists, nAlb-diABZI was intravenously administered to mice bearing ~75 mm³ tumors at doses ranging from 5-0.05 μg diABZI content, finding that all doses inhibited tumor growth and extended survival time. Notably, the 5 μg dose enhanced efficacy relative to a 3× higher dose of diABZI, demonstrating the enhancement in potency enabled through albumin-hitchhiking. While the 5 μg dose resulted in ~10-12% weight loss, this was transient and occurred only after the first injection (FIGS. 24A-24C). Nonetheless, towards maximizing the safety profile of the treatment, a dose of 1.25 μg was selected and a preclinical analysis of nAlb-diABZI toxicity was performed. Mice were administered vehicle (PBS) or nAlb-diABZI (1.25 μg diABZI) intravenously three times spaced three days apart, weight loss was monitored daily, and blood was collected 4 and 24 hr after the first injection for analysis of serum cytokines (FIGS. 9A-9E). In response to nAlb-diABZI, mice experienced only a mild (~5%) and transient weight loss similar to that described for nanoparticle-based delivery of STING agonists with elevated plasma levels of STING-driven cytokines with antitumor functions (e.g., type I IFN, TNF-α, etc.) 4 h following injection, which returned to near baseline by 24 h. Mice were euthanized a week following the last injection, blood was collected for biochemistry analysis, and major organs were isolated for histological evaluation by a board certified veterinary pathologist, who observed no clinically significant changes between the healthy control mice and nAlb-diABZI treated mice (FIGS. 9A-9E). Based on this favorable safety profile at a therapeutically effective dose in a rigorous B16.F10 tumor model, a dose of 1.25 μg for all subsequent studies was selected.

Given the significant tumor accumulation of nAlb observed in orthotopic EMT6 breast tumors—and considering that only approximately 20% of breast cancer patients benefit from PD-1/PD-L1 ICB39—the capacity of nAlb-diABZI to create a TME that inhibited tumor growth was evaluated next. Female Balb/c mice were inoculated with EMT6 cells in a mammary fat pad (MFP) and treated with nAlb-diABZI, free diABZI, or vehicle (PBS) at a tumor volume of ~75 mm³ (FIG. 3A). Noticeably, treatment with nAlb-diABZI strongly suppressed tumor growth whereas the free diABZI STING agonist did not confer a therapeutic benefit (FIGS. 3B-3C). Consistent with accumulation of nAlb at tumor sites, an increase in the expression of genes associated with STING pathway activation, including Ifnb1, Cxcl10, CxcI9, and Tnfa was observed (FIG. 10). To gain insight into the immunological mechanisms by which nAlb-diABZI inhibited tumor growth, multispectral flow cytometric immunophenotyping was used to quantify changes in key myeloid and lymphocyte populations and their phenotypes (FIG. 3D-J and FIG. 11) in EMT6 tumors and in the spleen 24 h following the third nAlb-diABZI administration. It was found that administration of nAlb-diABZI increased the infiltration of CD8⁺ T cells with elevated markers of activation (CD69) and proliferation (Ki67)—as well as the frequency of Ki67⁺PD-1⁺ CD8⁺ T cells—which have been correlated with favorable responses to immunotherapy in patients. While there was a reduction in the overall frequency of CD4⁺ T cells this was associated with an increased frequency of CD69⁺Ki67⁺ and Ki67⁺PD-1-CD4⁺ T cells. There was also a dramatic increase in the number of NK cells in the TME; interestingly, the levels of splenic CD69⁺ and K167⁺NK cells were also elevated, potentially suggesting mobilization of NKs from the spleen to the tumor (FIG. 25). An increase in MDSCs, the frequency of FoxP3⁺CD4⁺ T cells, and PD370 L1 on macrophages (FIG. 3H) was also observed, which may act as counter regulatory mechanisms that may contribute to resistance to Alb1-diABZI as a monotherapy.

Engineering an albumin-binding, bivalent nanobody fusion for combined STING agonist delivery and immune checkpoint inhibition. Having demonstrated the potent antitumor effects of the albumin hitchhiking STING agonist, the modularity of nanobody engineering was leveraged to confer additional immunotherapeutic functionality and demonstrate the programmability of the platform. As a translationally-relevant example, a second nanobody domain was introduced that binds to PD-L1 (anti-programmed cell death ligand 1). The rationale for selecting PD-L1 was two-fold. First, synergy between STING agonists and PD1/PD-L1 ICB in suppressing tumor growth, including evidence that STING activation can directly upregulate PD383 L1 expression, has been demonstrated. Second, PD-L1 can be expressed by both cancer cells and immunosuppressive myeloid cells in solid tumors, providing a molecular target for increasing tumor tropism. It was therefore hypothesized that an anti-albumin/anti-PD-L1 nanobody fusion would increase tumor targeting, while inhibiting immunoregulatory PD1/PD-L1 interactions that restrain responses to STING agonists. Thus, a fusion protein was generated that uses a genetic linker to connect both nanobody domains and maintained the C-terminal sortase ligation tag to generate an anti-albumin/anti-PD-L1 (AP)-STING agonist conjugate, termed AP-diABZI (FIG. 4A). The synthesis and generation of both anti-PD-L1 nanobody (nPD-L1) and AP conjugates to Cy5 and diABZI were characterized, showing that a single, homogeneous product that contained all three functional elements was formed (FIGS. 4B-4C, FIG. 26). The in vitro activity of nPD-L1-diABZI and AP-diABZI was tested in A549-Dual and THP1-Dual IFN-I reporter cells (FIGS. 4D-4E) and by qPCR for analysis of STING-associated cytokines/chemokine gene expression in primary murine myeloid cells (bone marrow derived macrophages, BMDMs; bone marrow derived dendritic cells, BMDCs) (FIG. 4F, FIGS. 27A-27D, and FIG. 28). It was found that all nanobody-diABZI conjugates were potently active in both reporter cell lines without evidence of cytotoxicity (FIGS. 27A-27D). Notably, increased activity of AP-diABZI relative to nAlb-diABZI or free diABZI in PD-L1-expressing BMDMs was also observed (FIG. 4F). Additionally, using flow cytometry, it was demonstrated that the incorporation of the PD-L1 targeting domain enhanced binding and internalization in B16.F10 (PD-LIlow) and EMT6 (PD-Lihigh) cells (FIGS. 4G-4H) relative to the albumin binding nanobody domain alone.

Next, the hypothesis that integrating a PD-L1 binding domain would increase tumor accumulation was tested. 2 mg/kg of Cy5-conjugated nEGFR, nPD-L1, nAlb, and AP nanobodies were administered to healthy Balb/c mice I.V. and collected blood at discrete time points to evaluate pharmacokinetics (FIG. 4I). Cy5-conjugated nanobodies were also administered to mice with orthotopic EMT6 breast tumors and euthanized mice at 48 h to quantify nanobody-Cy5 conjugate biodistribution to major organs and tumors using IVIS (FIGS. 4J-4M). While the AP-Cy5 conjugate had a shorter elimination half-life than nAlb-Cy5 (17 h to 55 hours, respectively), likely due to binding of target PD-L1 in tissue and removal from circulation, both carriers maintained an increased elimination half-life and AUC relative to either targeted nanobody (nEGFR and nPD443 L1) alone, which were cleared rapidly from circulation (FIG. 4I). While AP is approximately twice the size (~28 kDa) of the anti-PD-L1 nanobody, both are below the threshold for renal clearance and, therefore, the increased circulation time of AP can be primarily attributed to the albumin hitchhiking functionality. Further, while the nPD-L1-Cy5 conjugate was observed at similarly low levels in major organs (liver and kidneys) and the tumor at 48 h (FIG. 4J), the AP-Cy5 conjugate demonstrated significant tumor accumulation relative to major organs (FIG. 4K). Additionally, AP was found to increase tumor accumulation of Cy5-3-fold over nAlb alone (FIGS. 4L-4M), supporting the hypothesis that integrating a PD-L1 binding domain could further improve delivery to tumors.

AP-diABZI reprograms the TME to eliminate breast tumors and generate immunological memory that prevents recurrent disease. Next, the anti-tumor effects of systemically administered AP-diABZI fusion in the orthotopic EMT6 tumor model was investigated, comparing effects to those elicited by the constitutive components nAlb-diABZI and nPD-L1-diABZI (FIG. 5A). All nanobody carriers were administered I.V. at 1.25 μg of agonist. Additionally, mice were treated with commercially available anti-PD-L1 immune checkpoint blockade (ICB) IgG antibody to model 458 an FDA-approved anti-PD-L1 ICI (e.g., Atezolizumab). A standard preclinical dose of 100 μg 1C1 delivered intraperitoneally was used, which is a near equivalent molar dose of administered nanobody based on antigen binding domains. Remarkably, treatment with AP-diABZI completely eliminated observable EMT6 tumors, resulting in a 100% complete response (CR) rate (10/10 mice) whereas treatment nAlb-diABZI, while still very effective, yielded a 30% CR rate (3/10 mice) and nPD-L1-diABZI only modestly inhibited tumor growth, though to greater degree than the conventional anti-PD-L1 IgG ICB, which conferred only mild activity in this model. Importantly, no additional toxicity was observed for AP466 diABZI relative to nAlb-diABZI (FIGS. 9A-9E). AP-diABZI was also compared to a combination regimen of nAlb-diABZI and ICB (i.e., anti-PD-L1 IgG) and observed comparable antitumor responses, suggesting that the improved efficacy of AP-di ABZI over nAlb-diABZI can primarily be attributed to immune checkpoint inhibition. Mice treated with AP-diABZI and nAlb470 diABZI⁺ ICB that exhibited complete responses were rechallenged 80 days after the initial tumor inoculation with the injection of EMT6 cells in a distal MFP and tumor growth monitored without additional treatment. In both groups, mice were largely resistant to tumor re-challenge with only 1/9 (AP-diABZI) or ⅛ (nAlb-diABZI⁺ ICB) mice developing a tumor with the others remaining cancer free until at least day 100, demonstrating induction of memory lymphocytes that recognize EMT6 tumor antigens (FIGS. 5E-5F).To gain insight into the mechanism underlying the increased efficacy of AP-diABZI, mice bearing orthotopic EMT6 tumors were treated with AP-diABZI, nAlb-diABZI, or PBS, collected serum at 4 h following the first dose for analysis of serum cytokines (FIG. 5G), and euthanized mice 24 h after the third dose for gene expression analysis of tumor tissue using the NanoString PanCancer IO 360™ panel (FIGS. 5H-5L and FIGS. 12A-12C). Administration of nAlb-diABZI and AP-diABZI increased serum levels of antitumor IFN-1 (IFN-α, IFN-β) and Th1 cytokines (e.g., IL-12, TNF-α) whereas nPD-L1-diABZI did not stimulate response, consistent with its low therapeutic efficacy; interestingly, only AP-diABZI increased levels of IFN-γ, a cytokine with an established role in antitumor immunity. Likewise, both nAlb-diABZI and AP-diABZI mediated significant shifts in the gene expression profile, with transcript signatures associated with increased immune cell infiltrate (immune cell trafficking, CD8⁺ T cells, NK cells, Th1 cells), tumor immunogenicity (antigen presentation, T cell priming, T cell recognition, costimulation, cytokine/interferon signaling), and cancer cell death/apoptosis, with AP-diABZI tending to exert a stronger effect relative to nAlb-diABZI.

To further understand how AP exerts potent antitumor effects, flowcytometric immunophenotyping of EMT6 tumors was performed 48 h following the first dose of nAlb-diABZI and AP-diABZI (FIGS. 13A-13F). A decreasing frequency of live cancer cells (CD45⁻) was observed within the tumor and found a significant (P<0.0001) decrease in proliferating (K16⁺) cancer cells, consistent with the potent antitumor effects induced by AP-diABZI as well as gene expression analysis supporting increased cancer cell death. Interestingly, there was also an observed trend towards a decrease in PD-L1 expression within cancer cells. It was found that a single dose of either nAlb-diABZI or AP-diABZI increased the infiltration of neutrophils and NK cells; more granulocytic MDSCs were also present, potentially contributing as an immunoregulatory mechanism to acute STING activation. While no change in the overall frequency of CD8⁺ T cells was observed at this early time point, tumor infiltrating CD8⁺ T cells tended to display a more activated phenotype (i.e., CD69⁺), which was also reflected in the splenic T cell population (FIGS. 14A-14B). Motivated by these data, the tumor and spleen immune cell dynamics were studied after treatment with one, two, or three doses of AP-diABZI (FIG. 6, FIG. 15, and FIGS. 16A-16B). It was found that AP-diABZI increased the frequency of CD4⁺ T cells, CD8⁺ T cells and NK cells expressing markers of activation and proliferation, with a trend towards a stronger response after two and three doses, where a robust antitumor effect was observed (FIGS. 6A-6C). The frequency of tumor infiltrating T cells that were CD8⁺ T cells increased, with similar trends towards a more activated phenotype; and importantly, the ratio of CD8⁺ cells to FoxP3⁺ regulatory T cells was increased (FIG. 6D), indicative of a more immunogenic “hot” immune profile within the TME. Further, within both CD8⁺ and CD4⁺ T cells—both within the tumor and spleen—a shift towards Ki67⁺CD69⁺ and Ki67⁺PD-1 cells was observed, indicating the prevalence of both proliferating and activated lymphocytes in response to AP-diABZI (FIGS. 6E-6F). Consistent with observations following a single dose and the potent anti-tumor efficacy of AP-diABZI, the frequency of CD45-Ki67⁺ cancer cells was also reduced (FIG. 29) This is also consistent with gene expression profiling (FIGS. 5J-5K) indicating increased NK and T cell infiltration and tumoricidal activity. Together, these data demonstrate that AP-diABZI increases the infiltration of CD8⁺ T cells and NK cells with an activated phenotype and that this effect is enhanced over the use of nAlb-diABZI alone, potentially implicating CD8~T cells and NK cells as the primary antitumor effectors.

AP-diABZI inhibits B16.F10 tumor growth and primes an antigen-558 specific memory CD8⁺ T cell response in sitf. Next, the efficacy of AP-diABZI was assessed in a more challenging and immunosuppressive B16.F10 melanoma model, initiating the three-dose treatment regimen when subcutaneous tumors reached an average size of ~75 mm³. As expected in this model, anti-PD563 L1 ICB exerted no therapeutic benefit, whereas both nAlb-diABZI and AP-diABZI suppressed tumor growth and elongated median survival, with AP-diABZI conferring the most survival benefit, consistent with findings in the EMT6 model (FIGS. 7A-7D). Cytokine levels in plasma were also evaluated 4 h following the first injection using a multiplexed ELISA and found that anti-PD-L1 ICB increased only IL-1a levels, while nAlb-diABZI and AP-diABZI stimulated the production of cytokines associated with antitumor immunity, including IFN-α, IFN-β, IFN-γ, IL12p70, and CXCL10 (FIG. 7E, FIG. 17).

STING activation can prime the immune system to stimulate a systemic, antigen-specific, antitumor T cell responses with potential to lead to generation of T cell memory. Given evidence of increased antigen presentation, cancer cell killing, and T cell priming (FIGS. 5A-5L), as well as protection from tumor re-challenge in mice with EMT6 tumors treated with AP-diABZI, next, the capacity of AP-diABZI to stimulate a de novo tumor antigen-specific CD8⁺ T cell response was assessed. To test this, C57BIU6 mice was inoculated with B16.F10 melanoma cells expressing ovalbumin (B16.F10-OVA) as a model antigen and treated mice with either PBS or AP-diABZI on a three-dose regimen once tumors reached a size of 75-100 mm³ (FIG. 7F). 24 h after the final dose, mice were euthanized for flow cytometric evaluation of splenic T cell response. Consistent with results in mice with parental B16.F10 tumors, AP-diABZI treatment reduced tumor burden (FIG. 7G). Treatment with AP-diABZI resulted in a significant increase in activated CD69⁺, CD4~, and CD8⁺ T cells (FIG. 7I) and effector memory (CD44⁺CD62L~) CD4⁺ and CD8⁺ T cells, with a reduction in CD4⁺ central memory (CD44⁺CD62L⁺) T cells. Using SIlNFEKL/H-2 Kb tetramer staining, it was also found that AP-diABZI treatment stimulated a strong peripheral OVA specific CD8⁺ T cell response (FIG. 7M), characterized by a predominantly (~—60%) CD44⁺CD62L⁻ effector memory phenotype (FIG. 7N, FIG. 30). Hence, in addition to remodeling the TME, systemic administration of AP-diABZI primes antigen-specific CD8⁻ T cell effector and memory responses capable of targeting tumor-associated antigens.

Albumin-hitchhiking STING agonists inhibit lung metastatic disease. Based on the evidence that AP-diABZI can stimulate an effective antitumor immune response in the immunologically “cold” B16.F10 model, the investigations were extended to evaluate therapeutic efficacy in an aggressive model of lung metastatic melanoma induced through intravenous inoculation of luciferase-expressing B16.F10 (B16.F10-Luc) cells (FIG. 8A). A week following inoculation, the three-dose combination therapy regimen, described previously, was used. On day 17 post-inoculation, mice were euthanized, and lungs were harvested for quantification of tumor burden via measurement of lung mass, immunohistochemistry, and bioluminescent imaging (FIGS. 8B-8E). High metastatic tumor burden was evident in mice receiving anti-PD-L1 ICB alone, but reduced in mice receiving nAlb-diABZI and nearly eliminated in mice receiving AP-diABZI. Importantly, these data show that albumin-hitchhiking STING agonists are effective against metastases in the lung, one of the most common metastatic sites for many cancers (FIG. 8F). This also suggests a potential to treat micrometastases, which typically lack the leaky vasculature required for tumor accumulation via the enhanced permeation and retention effect; by contrast, albumin-binding molecules have been shown to accumulate in micrometases.

AP-diABZI opens a therapeutic window for adoptive T cell transfer therapy. Finally, to demonstrate the versatility of the platform, the application of AP-diABZI was extended to the setting of cellular immunotherapy, which includes CAR and TCR engineered T cells that face major barriers to tumor infiltration and function, which continue to limit their clinical impact in the treatment of solid tumors.51, 52 Founded on data demonstrating that nAlb-diABZI and AP-diABZI enhance the infiltration of endogenous antitumor T cells, it was hypothesized that the approach could be used to pre-condition the TME to generate a therapeutic window for adoptive T cell therapy. To test this, female C57BL/6 mice were inoculated with subcutaneous Bi6.F10-OVA cells and allowed the tumors to reach approximately 75 mm³ (FIG. 8G). The mice were then treated with either one or three doses of AP-diABZI, followed by a single intravenous dose of activated OVA-specific activated CD8⁺ T cells (OT-I T cells). Treatment with OT-I cells only (no STING agonist) on day 9 resulted in marginal therapeutic benefit (FIGS. 8H-8I), consistent with the highly immunosuppressive BI6.F10 TME that restricts T cell infiltration and effector function. However, treatment with OT-I T cells 48 h after either one or three AP-diABZI doses conferred significant reduction in tumor growth and prolonged mouse survival (FIGS. 8J-8K). Importantly, the treatment regimen of three doses of AP-diABZI prior to one dose of OT-I T cells resulted in a 25% complete response rate (3/12 mice). This observation provides additional evidence that albumin-hitchhiking STING agonists can establish an inflammatory milieu that supports T cell infiltration and function. While here a simplified model of an adoptive T cell therapy was employed, these studies highlight the potential to leverage nanobody-STING agonist conjugates to enhance responses to multiple T cell-based immunotherapies, including autologous tumor infiltrating lymphocyte (TIL) therapy, CAR T cells, and cancer vaccines.

Anti-B7H3 conjugates. In addition to PD-L1, conjugates were also made that targeted B7H3. Anti-Alb1/anti-B7H3 conjugates were made as described and shown in FIG. 35A, FIG. 35B, and FIG. 35C. Successful synthesis of anti-Alb1/anti-B7H3 conjugates is shown in FIG. 36A and FIG. 36B. In addition, anti-Alb1/anti-B7H3 conjugates were assessed in a tumor model (FIG. 37A, FIG. 37B, and FIG. 37C).

It is understood that the foregoing detailed description and accompanying examples are merely illustrative and are not to be taken as limitations upon the scope of the invention.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, various aspects of the invention are set out in the following numbered clauses:

Clause 1. A conjugate comprising: a nanobody portion, the nanobody portion comprising an albumin-binding nanobody, wherein the albumin-binding nanobody is capable of specifically binding albumin; a drug, and a linker attaching the nanobody portion to the drug, the linker comprising a nanobody linking moiety attached to the nanobody portion, the nanobody linking moiety including an amino acid sequence of LPXT (SEQ ID NO: 1). wherein X is any amino acid.

Clause 2. The conjugate of clause 1, the nanobody portion further comprising a second nanobody, wherein the second nanobody is capable of specifically binding an immune checkpoint ligand.

Clause 3. The conjugate of clause 2, wherein the immune checkpoint ligand is 72 CTLA-4, PD-1, PD-L1, B7-H3, B7-H4, HVEM, GITRL, CD80/86, CD155, PD-L1, Galectin 9, LAG3, TIM3, VISTA, TIGIT, PD1, or GITR

Clause 4. The conjugate of any one of clauses 1-3, wherein the linker further comprises a polyethylene glycol (PEG) moiety attaching the nanobody linking moiety to the drug.

Clause 5. The conjugate of any one of clauses 1-4, wherein the albumin-binding nanobody has a binding affinity (K_{d}) to albumin of less than or equal to 100 nM at a pH of about 7 to about 8.

Clause 6. The conjugate of any one of clauses 1-5, wherein the albumin-binding nanobody comprises an amino acid sequence selected from the group consisting of SEQ ID NO: 5 to SEQ ID NO: 79 and SEQ ID NO: 89.

Clause 7. The conjugate of any one of clauses 1-6, wherein the drug comprises an immunomodulator, an agonist, an antagonist, an inhibitor, or a hormone.

Clause 8. The conjugate of clause 7, wherein the immunomodulator comprises a STING agonist.

Clause 9. The conjugate of clause 8, wherein the STING agonist is a dimeric amidobenzimidazole, 2′3′ cGAMP, 2′2′ cGAMP, 3′2′ cGAMP, 3′3′ GAMP, c-di-GMP, c-di-AMP, ADU-S100, cIAMP 2-5, or ML RR-S2 CDA.

Clause 10. The conjugate of clause 8, wherein the immunomodulator is a non-nucleotide STING agonist.

Clause 11. The conjugate of clause 4, wherein the PEG moiety is of formula (a) or (b):

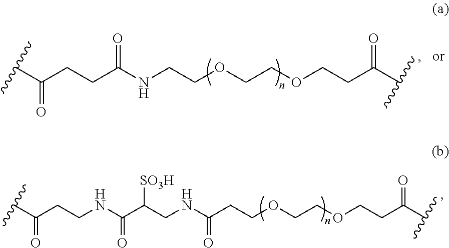

wherein: n is 2 to 20.

Clause 12. The conjugate of clause 11, wherein the PEG moiety is of formula (a) and n is 11.

Clause 13. The conjugate of clause 11, wherein the PEG moiety is of formula (b) and n is 3.

Clause 14. The conjugate of any one of clauses 1-13, wherein a drug to nanobody portion ratio is 1:1.

Clause 15. A method of preparing a conjugate, the method comprising:
reacting sortase with a nanobody portion, the nanobody portion including at least one nanobody having a sortase recognition site, to form a sortase-nanobody reagent, wherein the sortase-nanobody reagent comprises
   the nanobody portion,
   a nanobody linking moiety attached to the nanobody portion, the nanobody
   linking moiety including an amino acid sequence of LPXT (SEQ ID NO: 1), wherein X is any amino acid, and
   a sortase moiety attached to the nanobody linking moiety;
reacting the sortase-nanobody reagent with a reactive amine reagent, the reactive amine reagent comprising a first reactive group, to form a reactive nanobody reagent, the reactive nanobody reagent comprising
   the nanobody portion,
   the nanobody linking moiety, and
   a first reactive group attached to the nanobody linking moiety; and
coupling the reactive nanobody reagent with a reactive drug reagent, the reactive drug reagent comprising a drug and a second reactive group, to form a conjugate, the conjugate comprising the nanobody portion attached to the drug by a linker, the linker comprising the nanobody linking moiety.

Clause 16. The method of clause 15, the at least one nanobody comprising a first nanobody, wherein the first nanobody is capable of specifically binding albumin, CD63, EGFR, HER2, MMR, CD16, CTLA-4, CD33, PD-L1, B7-H3, fibronectin, or CD19.

Clause 17. The method of clause 16, wherein the first nanobody is capable of specifically binding albumin.

Clause 18. The method of any one of clauses 15-17, the at least one nanobody further comprising a second nanobody, wherein the second nanobody is capable of specifically binding an immune checkpoint ligand.

Clause 19. The method of clause 18, wherein the immune checkpoint ligand is 72 CTLA-4, PD-1, PD-L1, B7-H3, B7-H4, HVEM, G1TRL, CD80/86, CD155, PD-L1, Galectin 9, LAG3, TIM3, VISTA, TIGIT, PD1, or GITR

Clause 20. The method of any one of clauses 15-19, wherein the reactive amine reagent is of formula (I):

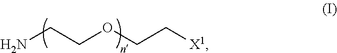

wherein: X¹ is the first reactive group and n′ is 2 to 20.

Clause 21. The method of any one of clauses 15-20, wherein the first reactive group comprises an azide, an alkyne, an alkene, a 1,2,4,5-tetrazine, or a thiol.

Clause 22. The method of any one of clauses 15-21, wherein the reactive nanobody reagent is of formula (II):

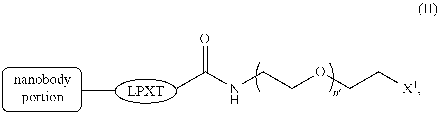

wherein: X¹ is the first reactive group, X is any amino acid, and n′ is 2 to 20.

Clause 23. The method of clause 22, wherein X¹ comprises an azide, an alkyne, an alkene, a 1,2,4,5-tetrazine, or a thiol.

Clause 24. The method of clause 23, wherein X¹ comprises an azide.

Clause 25. The method of any one of clauses 15-24, wherein the reactive drug reagent is of formula (III-a) or (III-b):

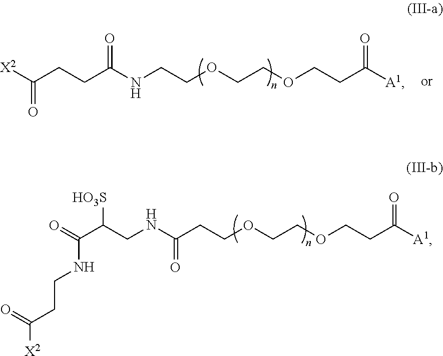

wherein: A¹ is the drug, X² is the second reactive group, and n is 2 to 20.

Clause 26. The method of clause 25, wherein X² comprises an alkyne, an azide, an alkene, a 1,2,4,5-tetrazine, or a thiol.

Clause 27. The method of clause 26, wherein X² comprises an alkyne.

Clause 28. The method of clause 27, wherein X² is

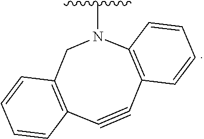 [SMILES: *N1Cc2ccccc2C#Cc2ccccc21]

Clause 29. The method of clause 25, wherein the reactive drug reagent is of formula (III-a) and n is 11.

Clause 30. The method of clause 25, wherein the reactive drug reagent is of formula (III-b) and n is 3.

Clause 31. The method of any one of clauses 15-30, wherein the drug comprises an immunomodulator, an agonist, an antagonist, an inhibitor, or a hormone.

Clause 32. The method of clause 31, wherein the immunomodulator comprises a STING agonist.

Clause 33. The method of clause 32, wherein the STING agonist is a dimeric amidobenzimidazole, 2′3′ cGAMP, 2′2′ cGAMP, 3′2′ cGAMP, 3′3′ cGAMP, c-di-GMP, c-di-AMP, ADU-S100, cIAMP 2-5, or ML RR-S2 CDA.

Clause 34. The method of clause 32, wherein the immunomodulator is a non-nucleotide STING agonist.

Clause 35. The method of any one of clauses 15-34, wherein a drug to nanobody portion ratio is 1:1.

Clause 36. The method of any one of clauses 25-35, wherein A¹ is:

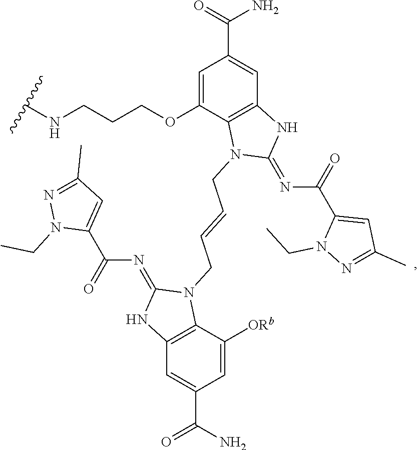

wherein:
R^{b} is methyl,

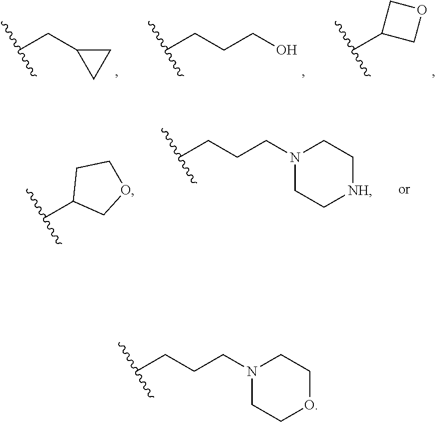

Clause 37. The method of clause 36, wherein R^{b} is methyl.

Clause 38. A pharmaceutical composition comprising: one or more conjugates according to any one of clauses 1-14; and a pharmaceutically acceptable excipient.

Clause 39. A method of treating a disease or a disorder in a subject in need thereof, the method comprising administering to the subject an effective amount of one or more conjugates according to any one of clauses 1-14, optionally in combination with a pharmaceutically acceptable excipient.

Clause 40. The method of clause 39, wherein the disease is a cancer, a viral infection, or multiple sclerosis.

Clause 41. The method of clause 39 or 40, wherein the cancer is melanoma, breast cancer, neuroblastoma, renal cell carcinoma, colon cancer, lung cancer, glioma, glioblastoma, or pancreatic cancer.

### Sequences

(SEQ ID NO: 1)
LPXT
(SEQ ID NO: 2)
LPET
(SEQ ID NO: 3)
LPXTG
(SEQ ID NO: 4)
LPETGGHHHHHHEPEA

Further details of SEQ ID NO: 5 to SEQ ID NO: 75, such as CDRs, PI, hydrophobicity, etc., can be found in Shen et al., A resource of high-quality and versatile nanobodies for drug delivery, iScience, Volume 24, Issue 9, 24 Sep. 2021, which is incorporated by reference herein in its entirety.

Nanobody ID NO. 13
(SEQ ID NO: 5)
AHVQLVESGGELVQAGGSLRLSCAASGRTFSNYAMGWFRQAPGTEREFVAAISRSGGS
TYYADSVKGRFIISRDNAKNTVWLQMNMLKPEDTSVYYCAAAEGLASGSYDYAPPLKS
SWYDYWGQGTQVTVSEPKTPKGGCGGG
Nanobody ID NO. 18
(SEQ ID NO: 6)
AEVQLVESGGGLAQAGGSLRLSCAASGRTFSNECLGWFRQAPGKEREFVATIRSTGHTS
YADAVSGRFTVSRDIAKNTVYLEMSNLKPEDTAVYSCAAGFSDYGCYRTSGINYWGQG
TQVTVSEPKTPKGGCGGG
Nanobody ID NO. 20
(SEQ ID NO: 7)
AQVQLVESGGGLVQAGGSLRLSCTASGRTFSSYYAMGWFRRAPGKEREFVAAISESGR
TTDYADSVKGRFTISRDTAKNTVYLQMISLKPEDTAVYYCAAAGPQEAFWFPSDYAQR
ALYDYWGQGTQVTVSEPKTPKGGCGGG
Nanobody ID NO. 23
(SEQ ID NO: 8)
AQVQLVESGGGLVQAGDSLRLSCAASERTFGAHVMGWFRQAPGKEREFVATITSSGRN
TRYADSVKGRFTISSDNAKNTVYLQMISLEPEDTAVYYCAYAYGAGLYNIARQYDYWG
QGTQVTVSEPKTPKGGCGGG
Nanobody ID NO. 27
(SEQ ID NO: 9)
AHVQLVESGGGLMQAGDSLRLSCAASGLTFSNYAMGWFRQAPGREREFVAALSWSGR
NGYYADSVKGRFTISSDNAKNTVYLQMNSLKPEDTAIYYCASAGGGGLYKIATQYDYW
GQGTQVTVSEPKTPKGGCGGG
Nanobody ID NO. 29
(SEQ ID NO: 10)
AQVQLVESGGGLVQPGGSLRLSCVASGIMFDIYTMRWYRQAPGKQRELVAAITGAGRA
NYNDDSVKGRFTISRDNAKNTVYLQMNRMKPEDTALYECNTEILGGGPNYWGRGTQV
TVSEPKTPKGGCGGG
Nanobody ID NO. 33
(SEQ ID NO: 11)
ADVQLVESGGGLAQAGGSLRLSCAASGRTFSNSCMGWFRQAPGKEREFVVTIRSTGHT
SYADAVSGRFTVSRDIAKNTVYLEMNSLKPEDTAVYSCGAGVSDYGCYHTSGYKYWG
QGTQVTVSEPKTPKGGCGGG
Nanobody ID NO. 35
(SEQ ID NO: 12)
AHVQLVESGGGLAQAGGSLRLSCAASGGTFSNSCMGWFRQAPGMEREFVATIRSTGHT
TYADSVEGRFTVSRDIAKNTVYLEMNSLKPEDTAVYSCGAGISDYGCYRTSGYNYWGQ
GTQVTVSEPKTPKGGCGGG
Nanobody ID NO. 36
(SEQ ID NO: 13)
AQVQLVESGGGLVQAGGSLRLSCRASGLPFGPYTMGWFRQTPGQEREFVAAITWSSMN
TNYADSVKGRFTISRDSAKNTVYLQMNTLKPDDTAVYYCAAAPGVGYYRHTFQYDYW
GQGTQVTVSEPKTPKGGCGGG
Nanobody ID NO. 39
(SEQ ID NO: 14)
AHVQLVESGGGLVQAGGSLRVSCAASGREFSNYGMGWFRQAPGKEREFVATISWNGRI
TFYADSVKGRFTISRDNAEKTGYLQMNSLKPEDTALYYCAAETSGWGSKVVPNYDYW
GQGTQVTVSEPKTPKGGCGGG
Nanobody ID NO. 46
(SEQ ID NO: 15)
AQVQLVESGGGLVQAGGSLRLSCTIPGHTISSYIMGWFRQAPGKEREFVAAINWSGGRT
NSADSVKGRFTISRDNAKNTVYLQMNSLKPEDTAVYYCAAVFDRISDSALPEERSRYDY
WGQGTQVTVSEPKTPKGGCGGG
Nanobody ID NO. 47
(SEQ ID NO: 16)
ADVQLVESGGGLAQAGGSLRLSCAASGRTFSNSCMGWFRQAPGKEREFVATIRSTGHIT
YADSVEGRFTVSRDIAKNTVYLEMSNLKPEDTAVYSCGAGVSDYGCYRTSGYNYWGQ
GTQVTVSEPKTPKGGCGGG
Nanobody ID NO. 48
(SEQ ID NO: 17)
AQVQLVESGGGLVQAGASLRLSCAASGGTFSSYIMGWFRQAPGREREFVAAISWSGRST
HYADSVKGRFAISRDNDRVYLQMDSLKPEDTAVYSCAADPNYTWRDDRYYREEGYTY
WGQGTQVTVSEPKTPKGGCGGG
Nanobody ID NO. 53
(SEQ ID NO: 18)
AQVQLVESGGGLVQSGGSLRLSCAASGSIGVTNTMGWYRQAPGKQRELAATITNDGNT
NYADSVKGRFTISRDNAKNTVYLQMNSLKPEDTAVYYCAGAGKRIPVSTMGWANDNE
YEYWGRGTQVTVSEPKTPKGGGGG
Nanobody ID NO. 57
(SEQ ID NO: 19)
AQVQLVESGGGLVQAGGSLRVSCAASGSTFSNYGMGWFRQAPGKEREFVAVIAWIGG
KTDYSDSVKGRFTIFRDNAKNTVYLQMNSLKPEDTAVYYCAATSYGTISRRSEYEYGY
WGQGTQVTVSEPKTPKGGCGGG
Nanobody ID NO. 59
(SEQ ID NO: 20)
AQVQLVESGGGLVQAGGSLRLSCAASGRTFSTYHMGWFRQAPGKAREFVAAITENGGI
TYYADSVRGRFTISRDDARNTVYLQMGSLKPEDTAVYYCAASSALIGRKYFGNENYSW
GQGTQVTVSEPKTPKGGCGGG
Nanobody ID NO. 60
(SEQ ID NO: 21)
AQVQLVESGGGLVQAGGSLRLSCAASGDTFSTYGVAWFRQAPGKERELVAITPWMGSS
TYYADSVKGRFTISRDNAKNTVYLQMNSLKPEDTAVYYCAASSYGSISRRSDYEYGYW
GQGTQVTVSEPKTPKGGCGGG
Nanobody ID NO. 61
(SEQ ID NO: 22)
AEVQLVESGGGLVQAGGSLRLSCAASGDTFGTYGVAWFRQAPGKEREFVAVTPWMGS
NTYYADSMKGRFTISRDNAKNTVYLQMNSLKPEDTAVYYCAASSYGTVSRRSEYEYGY
WGQGTQVTVSEPKTPKGGCGGG
Nanobody ID NO. 64
(SEQ ID NO: 23)
AQVQLVESGGGLVQAGDSLRLSCAASGRTFETHAMGWFRQAPGKEREFVATITPSGRS
TSYGDSVKGRFTISSDNAKNTVYLQMNSLKPDDTAIYYCAFAYGVGLYKLARQYDYW
GQGTQVTVSEPKTPKGGCGGG
Nanobody ID NO. 66
(SEQ ID NO: 24)
AQVQLVESGGGLVQAGDSLRLSCAASGRTFSTHGTGWFRQAPGKEREFVATITSSGRST
SYADSTKGRFTISSDNAKNTVYLQMNSLKPEDTAIYYCAYAYGVGLYKFATQYDYWG
QGTQVTVSEPKTPKGGCGGG
Nanobody ID NO. 68
(SEQ ID NO: 25)
AQVQLVESGGGLVQAGSSLRLSCAASGSTFSSHGMGWFRQAPGKEREFVATVSLSGRT
TSYGDSVKDRFTISRDNAKNTVYLHMNSLKLEDTAVYYCAATSGGYYSRYAYDYYYW
GQGTQVTVSEPKTPKGGCGGG
Nanobody ID NO. 69
(SEQ ID NO: 26)
AQVQLVESGGGLVQAGDSLRLSCAASGRTFETHAMGWFRQAPGKEREFVATITPSGRS
TSYADSVKGRYTISSDNAKNTVYLQMDSLKPEDTAIYYCAFAYGVGLYKIARQYDYWG
QGTQVTVSEPKTPKGGCGGG
Nanobody ID NO. 75
(SEQ ID NO: 27)
AEVQLVESGGGLVQAGGSLRLSCAASGRTYSPLVMGWFRQAPGKEREFVATITPSGGSL
SYADSVTGRFTVSRDNAKKTVFLQMNSLKPEDTAIYYCAAAPGVGNYRYARQYDYWG
QGTQVTVSEPKTPKGGCGGG
Nanobody ID NO. 76
(SEQ ID NO: 28)
AQVQLVESGGGLVQAGGSLRLSCTASGRTFTPYTMGWFRQAPGKEREFAASILWSGKN
TDYADSVKGRFAISKDNAKNTVYLQMNKLKPEDTAVYYCATGDGLGFYRSVSQYDYW
GQGTQVTVSEPKTPKGGCGGG
Nanobody ID NO. 77
(SEQ ID NO: 29)
AQVQLVESGGGLVQAGGSLRLSCTASGRTYEPLVMGWFRQAPGKEREFVATITPSGGSL
SYADSVKGRFTVSRDNAKKTVYLQMNRLQPEDTAVYYCAAAPGVGNYRYTRQYDYW
GQGTQVTVSEPKTPKGGCGGG
Nanobody ID NO. 78
(SEQ ID NO: 30)
AQVQLVESGGGLVQAGGSLRLSCTASGRTFTPYTIGWFRQAPGKEREFVASILWSGINT
DYADSVKGRFAISRDNAKNAAYLQMSNLKPEDTAVYYCATGGGLGYYRSVSQYDYW
GQGTQVTVSEPKTPKGGCGGG
Nanobody ID NO. 80
(SEQ ID NO: 31)
AEVQLVESGGGLVQAGGSLRLSCTASGRTFTPYTMGWFRQAPGKEREFVASILWSGNN
RDYADSVKGRFAISRDNAKNTAYLQMTSLKPEDTAVYYCAAGDGLGFYRSVNQYDYW
GQGTQVTVSEPKTPKGGCGGG
Nanobody ID NO. 81
(SEQ ID NO: 32)
AQVQLVESGGGLVQAGGSLKLSCTASGRTFMPYTMGWFRQVPGKEREFVASVLWSGI
NTDYAESVKGRFAISKDNAKNTMYLQMNSLKPEDTAVYYCAAGDGLGYYRSVSQYDY
WGHGTQVTVSEPKTPKGGCGGG
Nanobody ID NO. 82
(SEQ ID NO: 33)
AHVQLVESGGGLVQAGGSLRLSCAASGRTFSTYHMGWFRQAPGKAREFVAAITESGGI
TYYADSVKGRFTVSRDNAKNTVDLQMNSLKPEDTAVYYCAAAPGVGAYRHATQYDY
WGQGTQVTVSEPKTPKGGCGGG
Nanobody ID NO. 85
(SEQ ID NO: 34)
AQVQLVESGGGLVQAGGSLRLSCVASGRTFEPFVMGWFRQAPGKEREFVATISWSGGS
LSYADSVKGRFTVSRDNAKNTVYLQMNSLKPEDTAVYYCAAAPGVGNYRYTFQYDY
WGQGTQVTVSEPKTPKGGCGGG
Nanobody ID NO. 86
(SEQ ID NO: 35)
AQVQLVESGGGLVQAGGSLRLSCTASGRTFTPYTMGWFRQTPGKEREFAASILWSGINT
DYADSVKGRFAISKDNAKNTVYLQMNSLKPEDTAVYYCAAAYGLGYYRSVSQYDYW
GQGTQVTVSEPKTPKGGCGGG
Nanobody ID NO. 88
(SEQ ID NO: 36)
AQVQLVESGGGLVQAGGSLRLSCTASGRTFTPYTMGWFRQAPGKEREFAASILWSGEN
TDYADSVKGRFAISRDGAKNTVYLQMNSLKPEDTAVYYCASGYGLGFYRSASQYDYW
GQGTQVTVSEPKTPKGGCGGG
Nanobody ID NO. 89
(SEQ ID NO: 37)
AQVQLVESGGGLVQAGGSLRLSCTASGRTFTPYTMGWFRQAPGKEREFAASILWSGINT
DYADSVKGRFAISRDNAKNTVYLQMNSLKPEDIGVYYCAAADGLGLYRFVSQYDDW
GQGTQVTVSEPKTPKGGCGGG
Nanobody ID NO. 90
(SEQ ID NO: 38)
AQVQLVESGGGLVQAGGSLRLSCTASGRTFTPYTMGWFRQAPGKEREFAASILWSGINT
DYADSVKGRFAISRDNAKNTVYLQMNSLKPEDTGVYYCATADGLGLYRFVSQYDYWG
QGTQVTVSEPKTPKGGCGGG
Nanobody ID NO. 91
(SEQ ID NO: 39)
AQVQLVESGGGLVQAGGSLRLSCAASGRTFSPLVMGWFRQAPGHEREFVATITPSGGSQ
SYADSVKGRFAVSRDNAKKTVYLQMNSLKPEDTAVYYCAAAPGVGIYRYTSQYDYWG
QGTQVTVSEPKTPKGGCGGG
Nanobody ID NO. 92
(SEQ ID NO: 40)
AQVQLVESGGGLVQPGKSLRLSCTTSGLPGSWYTLGWFRQVPGKEREFVASVLWSGIN
TDYADSVKGRFAISRNNAKNTMYLQMNSLKPEDTAVYYCAAGYGLGFYRSVSQYDY
WGHGTQVTVSEPKTPKGGCGGG
Nanobody ID NO. 93
(SEQ ID NO: 41)
AQVQLVESGGGLVQAGGSLRLSCSASGSTFSPFVIGWFRQAPGKEREFVGGVRPSGSQY
YSDSVKGRFTVSRDNAKNTVYLQMNSLKPEDTAVYYCAAAAGVGNYRHTWQYDYW
GQGTQVTVSEPKTPKGGCGGG
Nanobody ID NO. 96
(SEQ ID NO: 42)
AHVQLVESGGGLVQAGGSLRLSCAASGHTFGPYTMGWFRQTPGKEREFVAAITWSGTS
TNYADSVKGRFTISRDNAKNTAYLQMNSLKPEDTAVYYCAAGSGAGRTSMHTSMTTG
ARGPRSPSEPKTPKGGCGGG
Nanobody ID NO. 98
(SEQ ID NO: 43)
AQVQLVESGGGLVQAGDSLRLSCVASGRTFSTYHMGWFRQAPGKAREFVAAITQSGIT
YYADSVKGRFTISRDNAKNTAYLQMGSLQPEDTAVYYCAASPKLIGRIYFGNENYSWG
QGTQVTVSEPKTPKGGCGGG
Nanobody ID NO. 99
(SEQ ID NO: 44)
AQVQLVESGGGLVQAGGSLRLSCAASGRTFSTYHMGWFRQAPGKAREFVAAITQSGGI
TYYADSVKGRFTISRDDAKNTVYLQMGSLEPEDTAVYYCAASPTLIGRVYFGNENYSW
GQGTQVTVSEPKTPKGGCGGG
Nanobody ID NO. 100
(SEQ ID NO: 45)
AQVQLVESGGGLVQAGGSLRLSCAASGRTFSTYHMGWFRQAPGKAREFVAAITGSGGI
TYYADSVKGRFTISRDNAKNTVYLQMNSLKPEDTAVYYCAASVALIGRVYFGNENYSW
GQGTQVTVSEPKTPKGGCGGG
Nanobody ID NO. 101
(SEQ ID NO: 46)
AQVQLVESGGGLVQAGGSLRLSCAASGRTFSTYHMGWFRQAPGKEREFVAAITQSGGI
TYYADAMKGRFTISRDDAKNTVYLQMGSLKPEDTAVYYCVASPALIGRHYFGNENYS
WGQGTQVTVSEPKTPKGGCGGG
Nanobody ID NO. 102
(SEQ ID NO: 47)
AHVQLVESGGGLVQAGGSLRLSCAASERTFSTYHMGWFRQAPGKGREFVAAITPSGGV
TYYADNLKGRFTISGDNAKNTVYLQMTNLKPEDTAVYYCVASPALIGRVYFGNENYSW
GQGTQVTVSEPKTPKGGCGGG
Nanobody ID NO. 104
(SEQ ID NO: 48)
AEVQLVESGGGLAQAGGSLRLSCAASGRTFSNSCMGWFRQAPGKEREFVATIRSTGHA
SYADSVEGRFTVSRDIAKNTVYLEMNSLKPDDTAVYICGAGVSDYGCYRTSGYNSWGQ
GTQVTVSEPKTPKGGCGGG
Nanobody ID NO. 108
(SEQ ID NO: 49)
AQVQLVESGGGLAQAGGSLRLSCAASGGTFSNNCMGWFRQAPGMEREFVAIRSTGHT
TYADSVEGRFTVSRDIAKNTVYLEMNSLKPEDTAVYICAAGASDYGCYRTSGINYWGQ
GTQVTVSEPKTPKGGCGGG
Nanobody ID NO. 113
(SEQ ID NO: 50)
AHVQLVESGGGLVQTGGSLRLSCVASGGIFSNSCMGWFRQAPGMERQFVAIIRSTGHTT
YADSVEGRFTVSRDIAKNTVYLEMNSLKPEDTAVYYCAAGVSDYGCYRTSGINYWGQ
GTQVTVSEPKTPKGGCGGG
Nanobody ID NO. 117
(SEQ ID NO: 51)
AQVQLVESGGGLVQAGGSLRLSCAASGRTFSTYHMGWFHQAPGKAREFVAAITESGGI
TYYADSVKGRFTISRDIAKNTVNLEMNSLKPEDTAVYSCAAGISDYGCYRTSGIAYWGQ
GTQVTVSEPKTPKGGCGGG
Nanobody ID NO. 118
(SEQ ID NO: 52)
AQVQLVESGGGLVQAGGSLRLSCAASGFSFSSYGMGWFRQAPGKEREFVAAIGWIGSR
TSYADSVKGRFTISKDNAKNTVYLQMDSLRPEDTAVYTCAATSYLNPDSDYARSDRSY
GYRGQGTQVTVSEPKTPKGGCGGG
Nanobody ID NO. 120
(SEQ ID NO: 53)
AEVQLVESGGGLVQAGDSLRLSCVASGRTFSTYHMGWFRQAPGKAREFVAAITQSGIT
YYADSVKGRFTISRDNAKNTAYLQMGSLQPEDTAVYYCAASPLLIGRVYFGNEDYSWG
PQRISEATTGARGPRSPSEPKTPKGGCGGG
Nanobody ID NO. 122
(SEQ ID NO: 54)
AQVQLVESGGGLVQAGGSLRLSCAASGRTFSTYHMGWFRQAPGKAREFVAAITGSGGI
TYYADSVKGRFTISRDNAKNTVYLQMNSLKPEDTAVYYCAASAALIGRVYFGNEITPGA
RGPRSPSEPKTPKGGCGGG
Nanobody ID NO. 124
(SEQ ID NO: 55)
AQVQLVESGGGLVQAGGSLRLSCAASERTFSSYAMGWFRQGPGKEREFVAYIHWSGGR
TLVVDSVKGRFTISRDNTKNTMYLQMNSLKPADTAVYYCTADQYASTLLRGTGEYWG
QGTQVTVSEPKTPKGGCGGG
Nanobody ID NO. 125
(SEQ ID NO: 56)
AQVQLVESGGGLVQAGGSLRLSCAAPGDIFSMYVMGWFRQAPGKEREFVAYNHWSGG
RTLYADSVKGRFTISRDNSKNTMSLQMNSLRPEDTAVYYCTADQYASTLLRAAGEYWG
QGTQVTVSEPKTPKGGCGGG
Nanobody ID NO. 126
(SEQ ID NO: 57)
AQVQLVESGGGLVQAGGSLRLSCAASGLTFSNYVMGWFRQAPGKEREFVAYIHWSGG
RILYADSVKGRFTISRDNTKNTMYLQMNSLKPDDTAVYYCTADQYATTVLRAAGEYW
GQGTQVTVSEPKTPKGGCGGG
Nanobody ID NO. 129
(SEQ ID NO: 58)
AQVQLVESGGGLVQAGGSLRLSCVASGRTFSPYTTGWFRQAPGKEREFVAAITWSGRS
TNYAASVKGRFTISRDNAKNTVYLQMDSLKPEDTAVYYCSAGAGGGIYTIRGQYDYWG
QGTQVTVSEPKTPKGGCGGG
Nanobody ID NO. 132
(SEQ ID NO: 59)
AHVQLVESGGGLVQAGGSLRLSCAASGGTISNYGMGWLRQGPGKEREFVGSINWNGA
TTHYADSVKGRFIISRDNAKNTVYLQMNSLKPEDTGVYYCVAQFSVQPTLRTYDYGGQ
GTQVTVSEPKTPKGGCGGG
Nanobody ID NO. 133
(SEQ ID NO: 60)
AHVQLVESGGGLVQAGGSLRLSCAASGLTFRNYAMGWFRRAPGKERDFVAAISYSGGS
TDYADSVKGRFTISRDNAKNTVFLQMSSLKPEDTAVYYCAASPVVYGSLWFKRESYTY
WGQGTQVTVSEPKTPKGGCGGG
Nanobody ID NO. 135
(SEQ ID NO: 61)
ADVQLVESGGGLVQAGGSLRLSCRASGLPFGPYTMGWFRQTPGQEREFVAAITWSSMN
TNYADSVKGRFTISRDNAKNTVLLQMNSLKPEDTAVYYCAAAEGLASGSYDYFPPLKS
SWYDYWGQGTQVTVSEPKTPKGGCGGG
Nanobody ID NO. 136
(SEQ ID NO: 62)
AQVQLVESGGGLVQPGGSLRLSCAASGFTLDYYAIGWFRQAPGKEREGVGCISSSDGSP
TYADSVKGRFTISRDNAKNTVYLQMNSLKPEDTAVYYCATHFRSANSCFLYYYDMDYS
GKGTQVTVSEPKTPKGGCGGG
Nanobody ID NO. 138
(SEQ ID NO: 63)
AQVQLVESGGGLVQAGDSLRLSCAASGRTFVAHAMGWFRQAPGKERTFVAMITSSGLT
ISYADPVKGRFTISSDNAKNTVYLQMNSLKPEDTAIYYCAFAYGVGKYEIARQYDYWG
QGTQVTVSEPKTPKGGCGGG
Nanobody ID NO. 139
(SEQ ID NO: 64)
AQVQLVESGGGAVQAGGSLQLSCRASGRTFSPYVMGWFRQAPGKEREFVGLITWSGGT
SYADSVRGRFTASRDRVKNTVYLQMNSLKPEDTAVYYCAAAYGAGYYVHERQYDYW
GQGTQVTVSEPKTPKGGCGGG
Nanobody ID NO. 140
(SEQ ID NO: 65)
AQVQLVESGGGLVQAGGSLRLSCTIPGHTISSYIMGWFRQAPGKEREFVAAINWSGGRT
NSADSVKGRFTISRDNAKNTVYLQMNSLKPEDTAVYYCAAVFDRLSDSALPEERSRYD
YWGQGTQVTVSEPKTPKGGCGGG
Nanobody ID NO. 141
(SEQ ID NO: 66)
AEVQLVESGGGLAQAGGSLRLSCAASGGTFSNSCMGWFRQAPGMEREFVAIIRSTGHTT
YADSVEGRFTISRDNAKNTVYLEMNSLKPEDTAVYYCAAERWTGACSGAGLHLRSFTS
WGQGTQVTVSEPKTPKGGCGGG
Nanobody ID NO. 143
(SEQ ID NO: 67)
AQVQLVESGGGLVQAGGSLSVSCAASGRTFRSYVGWFRQAPGKERTFVAGIRWSAGDT
YYADSMKGRFTISRDSAKNTVYLQMNSLKPEDTAVYYCAAAGPQQAFWFPSDYAQRA
LYDYWGQGTQVTVSEPKTPKGGCGGG
Nanobody ID NO. 144
(SEQ ID NO: 68)
AHVQLVESGGGLVQAGGSLRLSCAASGRTFIPYTTGWFRQTPGKEREFVATITWSGISTK
FADSVKGRFTISRDNAKNTVYLQMNSLKPEDTAVYYCAASGPQEAFWFPSDYAQRALY
DYWGQGTQVTVSEPKTPKGGCGGG
Nanobody ID NO. 145
(SEQ ID NO: 69)
AQVQLVESGGGLVQAGGSLRLSCRASGLPFGPYTMGWFRQTPGQEREFVAAITWSSMN
TNYADSVKGRFTISRDSAKNTVYLQMNSLKREDTAVYYCAAAGPQEAFWFPSDYAQR
ALYDYWGQGTQVTVSEPKTPKGGCGGG
Nanobody ID NO. 147
(SEQ ID NO: 70)
AHVQLVESGGGLVQPGGSLRLSCVASGIMFDIYTMRWYRQAPGKQRELVAAITGAGRA
NYNDDSVKGRFTISRDNAKNTVYLQMNRMKPEDTALYECNTEILGGGRNYWGRGTQV
TVSEPKTPKGGCGGG
Nanobody ID NO. 148
(SEQ ID NO: 71)
AQVQLVESGGGLVQAGDSLRLSCAVSGRAFSNDIFGWFRQAPGLEREFVAAHRWNALY
ADSVKGRFTISRDNAKNTVYLQMNSLKPEDTAVYFCAGAGKRFPVSAMGWANDNEYE
YWGRGTQVTVSEPKTPKGGCGGG
Nanobody ID NO. 150
(SEQ ID NO: 72)
AHVQLVESGGGLVQAGGSLRLSCAVSGRTFSNDIIGWFRQAPGKDREFVAAHRYNALY
ADSVKGRFTISRDNAKNTVYLQMNSLKPEDTALYYCAGAGKRYPVSTMGWANDNEYE
YWGRGTQVTVSEPKTPKGGCGGG
Nanobody ID NO. 151
(SEQ ID NO: 73)
AQVQLVESGGGLAQAGGSLRLSCAASGGTFSNSCMGWFRQAPGMEREFVATIRSTGHT
TYADSVEGRFTVSRDIAKNTVYLEMNSLKREDTAVYTCAAGVSDYGCYHTSGYNYWG
QGTQVTVSEPKTPKGGCGGG
Nanobody ID NO. 154
(SEQ ID NO: 74)
AQVQLVESGGGLVQAGGSLRLSCTVPGHTISSYIMGWFRQAPGKEREFVAAINWNGGR
TNSADSVKGRFTISRDNAKNTVYLQMNSLKPEDTAVYYCAAVFDRISDSFLPEERSTYD
YWGQGTQVTVSEPKTPKGGCGGG
Nanobody ID NO. 158
(SEQ ID NO: 75)
ADVQLVESGGGLVQAGGSLRLSCAASGGTISNYGMGWLRQGPGKEREFVGSINWNGA
TTHYADSVKGRFIISRDNAKNTVYLQMNSLKPEDTGVYYCVAQFSVQPTLQTYDYRGQ
GTQVTVSEPKTPKGGCGGG
aAlbumin-eSrtA Tag (Alb1)
(SEQ ID NO: 76)
EVQLVESGGGLVQPGGSLRLSCAASGFTFRSFGMSWVRQAPGKEPEWVSSISGSGSDTL
YADSVKGRFTISRDNAKTTLYLQMNSLKPEDTAVYYCTIGGSLSRSSQGTQVTVSSLPET
GGHHHHHHEPEA
aAlbumin-eSrtA Tag (M75)
(SEQ ID NO: 77)
QVQLVESGGGFVQAGGSLRLSCAASGRTFDNYVMAWFRQAPGKEREFVASISGSGSITN
YANSVKDRFTISRDSAKNAIYLQMNSLKPEDTALYYCAAGSRRTYYREPKFYPSWGQG
TQVTVSSLPETGGHHHHHHEPEA
aAlbumin-mCherry-eSrtA Tag (Alb1)
(SEQ ID NO: 78)
EVQLVESGGGLVQPGGSLRLSCAASGFTFRSFGMSWVRQAPGKEPEWVSSISGSGSDTL
YADSVKGRFTISRDNAKTTLYLQMNSLKPEDTAVYYCTIGGSLSRSSQGTQVTVSSSGSE
TPGTSESAVSKGEEDNMAIIKEFMRFKVHMEGSVNGHEFEIEGEGEGRPYEGTQTAKLK
VTKGGPLPFAWDILSPQFMYGSKAYVKHPADIPDYLKLSFPEGFKWERVMNFEDGGVV
TVTQDSSLQDGEFIYKVKLRGTNFPSDGPVMQKKTMGWEASSERMYPEDGALKGEIKQ
RLKLKDGGHYDAEVKTTYKAKKPVQLPGAYNVNIKLDITSHNEDYTIVEQYERAEGRH
STGGMDELYKLPETGGHHHHHHEPEA
aAlbumin-mCherry-eSrtA Tag (M75)
(SEQ ID NO: 79)
QVQLVESGGGFVQAGGSLRLSCAASGRTFDNYVMAWFRQAPGKEREFVASISGSGSITN
YANSVKDRFTISRDSAKNAIYLQMNSLKPEDTALYYCAAGSRRTYYREPKFYPSWGQG
TQVTVSSSGSETPGTSESAVSKGEEDNMAIIKEFMRFKVHMEGSVNGHEFEIEGEGEGRP
YEGTQTAKLKVTKGGPLPFAWDILSPQFMYGSKAYVKHPADIPDYLKLSFPEGFKWER
VMNFEDGGVVTVTQDSSLQDGEFIYKVKLRGTNFPSDGPVMQKKTMGWEASSERMYP
EDGALKGEIKQRLKLKDGGHYDAEVKTTYKAKKPVQLPGAYNVNIKLDITSHNEDYTI
VEQYERAEGRHSTGGMDELYKLPETGGHHHHHHEPEA
eSrtA
(SEQ ID NO: 80)
QAKPQIPKDKSKVAGYIEIPDADIKEPVYPGPATREQLNRGVSFAEENESLDDQNISIAGH
TFIDRPNYQFTNLKAAKKGSMVYFKVGNETRKYKMTSIRNVKPTAVEVLDEQKGKDK
QLTLITCDDYNEETGVWETRKIFVATEVKLEHHHHHH
Anti-EGFR (nEGFR) Sequence (No Start Codon)
nEGFR-Ligation Tag
(SEQ ID NO: 81)
QVKLEESGGGSVQTGGSLRLTCAASGRTSRSYGMGWFRQAPGKEREFVSGISWRGDST
GYADSVKGRFTISRDNAKNTVDLQMNSLKPEDTAIYYCAAAAGSAWYGTLYEYDYWG
QGTQVTVSS-LPETGGHHHHHHEPEA
Anti-PD-L1 (nPD-L1) Sequence (No Start Codon)
nPD-L1-Ligation Tag
(SEQ ID NO: 82)
QVQLQESGGGLVHPGGSLRLSCATSGSIFSIISMGWYRQAPGKQRELVALVFRGGSTVY
ADSVKGRFTISGDIAKSTVYLQMDSLKPEDTAVYYCNAKPIGTAQYWGQGTQVTVSS-
LPETGGHHHHHHEPEA
Anti-Albumin-Anti-PD-L1 (AP) Sequence (No Start Codon)
nAlb-XTEN Linker-nPD-L1-Ligation Tag
(SEQ ID NO: 83)
EVQLVESGGGLVQPGGSLRLSCAASGFTFRSFGMSWVRQAPGKEPEWVSSISGSGSDTL
YADSVKGRFTISRDNAKTTLYLQMNSLKPEDTAVYYCTIGGSLSRSSQGTQVTVSS-
SGSETPGTSESA-
QVQLQESGGGLVHPGGSLRLSCATSGSIFSIISMGWYRQAPGKQRELVALVFRGGSTVY
ADSVKGRFTISGDIAKSTVYLQMDSLKPEDTAVYYCNAKPIGTAQYWGQGTQVTVSS-
LPETGGHHHHHHEPEA
XTEN
(SEQ ID NO: 84)
SGSETPGTSESA
Anti-Albumin-Anti-B7H3
(SEQ ID NO: 85)
MEVQLVESGGGLVQPGGSLRLSCAASGFTFRSFGMSWVRQAPGKEPEWVSSISGSGSDT
LYADSVKGRFTISRDNAKTTLYLQMNSLKPEDTAVYYCTIGGSLSRSSQGTQVTVSSSGS
ETPGTSESAQEQLKESGGRLVTPGTPLTLTCTVSGFSPNNYGVSWVRQPPGKGLEWIGM
SSTAGATYYANWAKGRFTISKTSTTVDLEITSPTTEDTATYFCAKGTPSLSYGNIWGPGT
LVTVSSLPETGGHHHHHHEPEA
Anti-Albumin-Anti-B7H3
(SEQ ID NO: 86)
Atggaagtgcagctggtggaaagcggcggcggcctggtgcagccgggcggcagcctgcgcctgagctgcgcggcgagcggctttac
ctttcgcagctttggcatgagctgggtgcgccaggcgccgggcaaagaaccggaatgggtgagcagcattagcggcagcggcagcgat
accctgtatgcggatagcgtgaaaggccgctttaccattagccgcgataacgcgaaaaccaccctgtatctgcagatgaacagcctgaaac
cggaagataccgcggtgtattattgcaccattggcggcagcctgagccgcagcagccagggcacccaggtgaccgtgagcagcagcgg
cagcgaaaccccgggcaccagcgaaagcgcgcaggaacagctgaaagaaagcggcggccgcctggtgaccccgggcaccccgctg
accctgacctgcaccgtgagcggctttagcccgaacaactatggcgtgagctgggtgcgccagccgccgggcaaaggcctggaatggat
tggcatgagcagcaccgcgggcgcgacctattatgcgaactgggcgaaaggccgctttaccattagcaaaaccagcaccaccgtggatct
ggaaattaccagcccgaccaccgaagataccgcgacctatttttgcgcgaaaggcaccccgagcctgagctatggcaacatttggggccc
gggcaccctggtgaccgtgagcagcctgccggaaaccggcggccatcatcatcatcatcatgaaccggaagcg
Anti-B7H3 (with ligation tag)
(SEQ ID NO: 87)
QEQLKESGGRLVTPGTPLTLTCTVSGFSPNNYGVSWVRQPPGKGLEWIGMSSTAGATY
YANWAKGRFTISKTSTTVDLEITSPTTEDTATYFCAKGTPSLSYGNIWGPGTLVTVSSLPE
TGGHHHHHHEPEA
Anti-B7H3 (no ligation tag)
(SEQ ID NO: 88)
QEQLKESGGRLVTPGTPLTLTCTVSGFSPNNYGVSWVRQPPGKGLEWIGMSSTAGATY
YANWAKGRFTISKTSTTVDLEITSPTTEDTATYFCAKGTPSLSYGNIWGPGTLVTVSS
Anti-Albumin (Alb1) (no ligation tag)
(SEQ ID NO: 89)
EVqLVESGGGLVQPGGSLRLSCAASGFTFRSFGMSWVRQAPGKEPEWVSSISGSGSDTL
YADSVKGRFTISRDNAKTTLYLQMNSLKPEDTAVYYCTIGGSLSRSSQGTQVTVSS
Anti-PD-L1 (nPD-L1) (no ligation tag)
(SEQ ID NO: 90)
QVQLQESGGGLVHPGGSLRLSCATSGSIFSIISMGWYRQAPGKQRELVALVFRGGSTVY
ADSVKGRFTISGDIAKSTVYLQMDSLKPEDTAVYYCNAKPIGTAQYWGQGTQVTVSS

What is claimed:

1. A conjugate comprising:
a nanobody portion, the nanobody portion comprising an albumin-binding nanobody, wherein the albumin-binding nanobody is capable of specifically binding albumin;
a drug; and
a linker attaching the nanobody portion to the drug, the linker comprising a nanobody linking moiety attached to the nanobody portion, the nanobody linking moiety including an amino acid sequence of LPXT (SEQ ID NO: 1), wherein X is any amino acid.

2. The conjugate of claim 1, the nanobody portion further comprising a second nanobody, wherein the second nanobody is capable of specifically binding an immune checkpoint ligand.

3. The conjugate of claim 2, wherein the immune checkpoint ligand is 72 CTLA-4, PD-1, PD-L1, B7-H3, B7-H4, HVEM, GITRL, CD80/86, CD155, PD-L1, Galectin 9, LAG3, TIM3, VISTA, TIGIT, PD1, or GITR.

4. The conjugate of claim 1, wherein the linker further comprises a polyethylene glycol (PEG) moiety attaching the nanobody linking moiety to the drug.

5. The conjugate of claim 1, wherein the albumin-binding nanobody has a binding affinity (K_{d}) to albumin of less than or equal to 100 nM at a pH of about 7 to about 8.

6. The conjugate of claim 1, wherein the albumin-binding nanobody comprises an amino acid sequence selected from the group consisting of SEQ ID NO: 5 to SEQ ID NO: 79 and SEQ ID NO: 89.

7. The conjugate of claim 1, wherein the drug comprises an immunomodulator, an agonist, an antagonist, an inhibitor, or a hormone.

8. The conjugate of claim 7, wherein the immunomodulator comprises a STING agonist.

9. The conjugate of claim 8, wherein the STING agonist is a dimeric amidobenzimidazole, 2′3′ cGAMP, 2′2′ cGAMP, 3′2′ cGAMP, 3′3′ cGAMP, c-di-GMP, c-di-AMP, ADU-S100, cIAMP 2-5, or ML RR-S2 CDA.

10. The conjugate of claim 8, wherein the immunomodulator is a non-nucleotide STING agonist.

11. The conjugate of claim 4, wherein the PEG moiety is of formula (a) or (b): 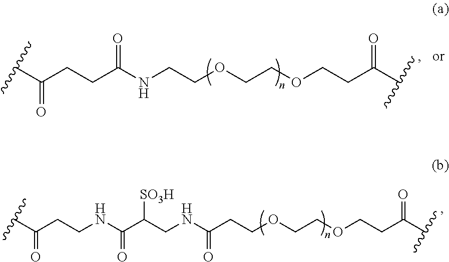
wherein:
n is 2 to 20.

12. The conjugate of claim 11, wherein the PEG moiety is of formula (a) and n is 11.

13. The conjugate of claim 11, wherein the PEG moiety is of formula (b) and n is 3.

14. The conjugate of claim 1, wherein a drug to nanobody portion ratio is 1:1.

15. A method of preparing a conjugate, the method comprising:
reacting sortase with a nanobody portion, the nanobody portion including at least one nanobody having a sortase recognition site, to form a sortase-nanobody reagent, wherein the sortase-nanobody reagent comprises
the nanobody portion,
a nanobody linking moiety attached to the nanobody portion, the nanobody linking moiety including an amino acid sequence of LPXT (SEQ ID NO: 1), wherein X is any amino acid, and
a sortase moiety attached to the nanobody linking moiety;
reacting the sortase-nanobody reagent with a reactive amine reagent, the reactive amine reagent comprising a first reactive group, to form a reactive nanobody reagent, the reactive nanobody reagent comprising
the nanobody portion,
the nanobody linking moiety, and
a first reactive group attached to the nanobody linking moiety; and
coupling the reactive nanobody reagent with a reactive drug reagent, the reactive drug reagent comprising a drug and a second reactive group, to form a conjugate, the conjugate comprising
the nanobody portion attached to the drug by a linker, the linker comprising the nanobody linking moiety.

16. The method of claim 15, the at least one nanobody comprising a first nanobody, wherein the first nanobody is capable of specifically binding albumin, CD63, EGFR, HER2, MMR, CD16, CTLA-4, CD33, PD-L1, B7-H3, fibronectin, or CD19.

17. The method of claim 16, wherein the first nanobody is capable of specifically binding albumin.

18. The method of claim 15, the at least one nanobody further comprising a second nanobody, wherein the second nanobody is capable of specifically binding an immune checkpoint ligand.

19. The method of claim 18, wherein the immune checkpoint ligand is 72 CTLA-4, PD-1, PD-L1, B7-H3, B7-H4, HVEM, GITRL, CD80/86, CD155, PD-L1, Galectin 9, LAG3, TIM3, VISTA, TIGIT, PD1, or GITR.

20. The method of claim 15, wherein the reactive amine reagent is of formula (I): 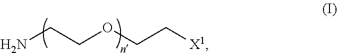
wherein:
X¹ is the first reactive group and n′ is 2 to 20.

21. The method of claim 15 or 20, wherein the first reactive group comprises an azide, an alkyne, an alkene, a 1,2,4,5-tetrazine, or a thiol.

22. The method of claim 15, wherein the reactive nanobody reagent is of formula (II): 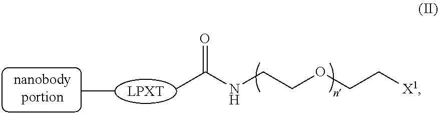
wherein:
X¹ is the first reactive group,
X is any amino acid, and
n′ is 2 to 20.

23. The method of claim 22, wherein X¹ comprises an azide, an alkyne, an alkene, a 1,2,4,5-tetrazine, or a thiol.

24. The method of claim 23, wherein X¹ comprises an azide.

25. The method of claim 15, wherein the reactive drug reagent is of formula (III-a) or (III-b): 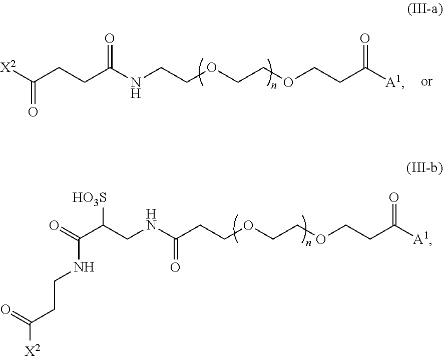
wherein:
A¹ is the drug,
X² is the second reactive group, and
n is 2 to 20.

26. The method of claim 25, wherein X² comprises an alkyne, an azide, an alkene, a 1,2,4,5-tetrazine, or a thiol.

27. The method of claim 26, wherein X² comprises an alkyne.

28. The method of claim 27, wherein X² is

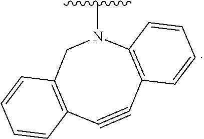 [SMILES: *N1Cc2ccccc2C#Cc2ccccc21]

29. The method of claim 25, wherein the reactive drug reagent is of formula (III-a) and n is 11.

30. The method of claim 25, wherein the reactive drug reagent is of formula (III-b) and n is 3.

31. The method of claim 15, wherein the drug comprises an immunomodulator, an agonist, an antagonist, an inhibitor, or a hormone.

32. The method of claim 31, wherein the immunomodulator comprises a STING agonist.

33. The method of claim 32, wherein the STING agonist is a dimeric amidobenzimidazole, 2′3′ cGAMP, 2′2′ cGAMP, 3′2′ cGAMP, 3′3′ cGAMP, c-di-GMP, c-di-AMP, ADU-S100, cIAMP 2-5, or ML RR-S2 CDA.

34. The method of claim 32, wherein the immunomodulator is a non-nucleotide STING agonist.

35. The method of claim 15, wherein a drug to nanobody portion ratio is 1:1.

36. The method of claim 25, wherein A¹ is: 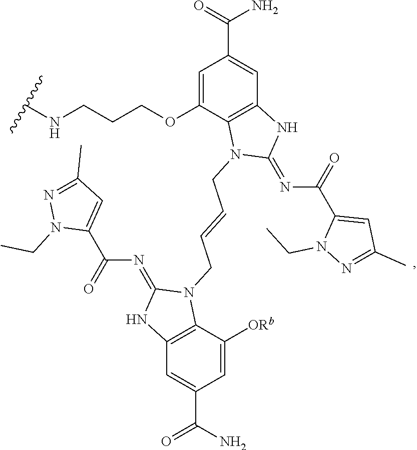
wherein:
R^{b} is methyl,

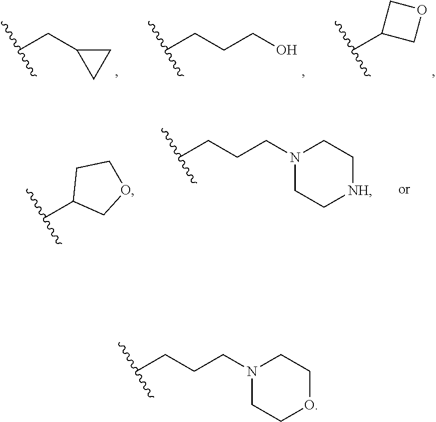

37. The method of claim 36, wherein R^{b} is methyl.

38. A pharmaceutical composition comprising:
one or more conjugates according to claim 1; and
a pharmaceutically acceptable excipient.

39. A method of treating a disease or a disorder in a subject in need thereof, the method comprising administering to the subject an effective amount of one or more conjugates according to claim 1, optionally in combination with a pharmaceutically acceptable excipient.

40. The method of claim 39, wherein the disease is a cancer, a viral infection, or multiple sclerosis.

41. The method of claim 39 or 40, wherein the cancer is melanoma, breast cancer, neuroblastoma, renal cell carcinoma, colon cancer, lung cancer, glioma, glioblastoma, or pancreatic cancer.
